(19) United States
(12) Patent Application Publication
(10) Pub. No.: US 2026/0355267 A1
(43) Pub. Date: Nov. 12, 2026

(54) BIOCONTROL COMPOSITIONS OF BACTERIOPHAGE, METHODS OF PRODUCING AND USES THEREOF

(71) Applicant: SUG BIOSCIENCES LLC, Wilmington, DE (US)

(72) Inventor: Josefina PUIG, Canelones (UY)

(73) Assignee: SUG BIOSCIENCES LLC, Wilmington, DE (US)

(21) Appl. No.: 19/123,333

(22) PCT Filed: Oct. 24, 2023
(86) PCT No.: PCT/US2023/077688

§ 371 Date: Apr. 22, 2025

Related U.S. Application Data
(60) Provisional application No. 63/419,187, filed on Oct. 25, 2022.

(51) Int. Cl. A61K 35/76; A61K 45/06; A61P 31/04; C12N 7/00

(52) U.S. Cl. CPC A61K 35/76; A61K 45/06; A61P 31/04; C12N 7/00; C12N 2795/00031; C12N 2795/00032

(57) ABSTRACT

The invention relates to a biocontrol composition comprising at least one distinct bacteriophage having substantial lytic activity against a wide range of pathogenic E. coli, their methods of preparation and uses in human and animal phage-based therapy and remediation of contaminated environment.

This application claims priority to U.S. Provisional Application No. 63/419,187, filed Oct. 25, 2022, the contents of which are incorporated herein by reference in their entirety.

### SEQUENCE LISTING

The instant application contains a Sequence Listing which has been submitted electronically in .XML format and is hereby incorporated by reference in its entirety. Said .XML file, created on Oct. 18, 2023, is named 151089_584969_SL.xml and is 1984158 bytes in size.

### FIELD OF THE INVENTION

The field of the currently claimed embodiments of this invention relate to novel bacteriophages and bacteriophage cocktails containing the novel bacteriophages and or parts and/or products of them that have a broad host-range against different isolates and strains of pathogenic bacteria such as Escherichia coli, and their application in phage therapy, animal therapy and environmental remediation. In particular, the invention relates to a biocontrol composition comprising at least one distinct bacteriophage having substantial lytic activity against at least one pathogenic bacterial strain.

### DISCUSSION OF RELATED ART

Bacteriophages (phages) are the most abundant entities on Earth. They are bacterial viruses composed of a nucleic acid (DNA or RNA) encapsulated by a protein coat (Clark J R, March J B: Bacteriophages and biotechnology: vaccines, gene therapy and antibacterials. Trends Biotechnol 2006, 24 (5): 212-218; Haq, I. U., Chaudhry, W. N., Akhtar, M. N. et al. Bacteriophages and their implications on future biotechnology: a review. Virol J 9, 9 (2012).) The protein coat, or capsid, is attached to a tail and a baseplate to which long tail fibers are attached. The first step of infection is recognition of receptor molecules or lipopolysaccharide (LPS) on bacterial surface by the tips of the long tail fibers and the attachment to the bacterium (See Haq, Ackerman H W: Tailed bacteriophages: the Caudovirales. Adv Virus Res 1998, 51:135-201.) The specific attachment process is thought to influence the spectrum of phage-bacteria interactions. Bacteriophages can infect bacteria in two different ways with different outcomes. In a lytic cycle, phages enter the bacterium, hijack the bacterial machinery to generate copies of themselves, and lyse the bacteria to release new phages. In a lysogenic cycle, phages enter the bacterium, and integrate their genomes into the bacterial chromosome, thus remaining latent for extended period of time and replicating as part of the bacterial chromosome. They can resume a lytic cycle after a lysogenic cycle. Bacteriophages that only infect through a lytic cycle are referred to as virulent, while those having the capacity to infect via a lytic or lysogenic cycle are referred to as temperate. (Clark, Haq)

Since the discovery of bacteriophages in the early twentieth century, there has been a great interest in their antibacterial potential. The advent of antibiotics in the 1930s and 1940s lead to a waning interest in the potential therapeutic applications of bacteriophages. (Murray C J, Ikuta K S, Sharara F, Swetschinski L, Aguilar G R, Gray A, et al. Global burden of bacterial antimicrobial resistance in 2019: a systematic analysis. The Lancet 2022) However, with the emergence of bacterial antimicrobial resistance (AMR) across multiple bacterial pathogens, bacteriophages have now become interesting alternatives to antibiotics for the treatment of bacterial infections.

Bacterial antimicrobial resistance (AMR), occurs when inherited changes in bacterial DNA cause a drug used to treat infections to become less effective. AMR is one of the leading public health threats of the 21^{st} century. Recent estimates revealed that in 2019 over 4.5 million people died globally due to complications caused by AMR infections. (Id.) Accordingly, the WHO and numerous groups of experts and researchers agree that the spread of AMR is an urgent medical challenge which requires a global action plan. (Global action plan on antimicrobial resistance n.d. https://www.who.int/publications-detail-redirect/9789241509763 (accessed Aug. 1, 2022).) This plan must be underpinned by the pillars of the One Health approach, which involves the collaborative effort of multiple disciplines to reach optimal health for people, animals and our environment. AMR has clear links to each of these three domains. Antibiotic use in humans is overshadowed by their use in animals, with two-thirds of overall antibiotic usage destined for livestock. (Done H Y, Venkatesan A K, Halden R U. Does the Recent Growth of Aquaculture Create Antibiotic Resistance Threats Different from those Associated with Land Animal Production in Agriculture? AAPS J 2015; 17:513-24.) Antibiotics used to promote animal growth has been banned in many countries. However, over 131,000 tons of antimicrobials were still used globally in the animal industry in 2013 and may rise to approximately 200,000 tons by 2030. (Van Boeckel T P, Glennon E E, Chen D, Gilbert M, Robinson T P, Grenfell B T, et al. Reducing antimicrobial use in food animals. Science 2017; 357:1350-2.)

The WHO has made a priority to limit the intensive antibiotic usage. (Tacconelli E, Carrara E, Savoldi A, Harbarth S, Mendelson M, Monnet D L, et al. Discovery, research, and development of new antibiotics: the WHO priority list of antibiotic-resistant bacteria and tuberculosis. Lancet Infect Dis 2018; 18:318-27.) These AMR pathogens are particularly relevant due to the wide range of environments and animals in which they thrive and their ability to acquire and exchange genetic elements that code for extended-spectrum beta lactamases (ESBLs) and carbapenemases, generating multiresistant (MDR) strains. Among them, MDR Escherichia coli is identified as a major cause of bloodstream, urinary tract and other invasive infections in both community and health care settings globally. (Cassini A, Högberg L D, Plachouras D, Quattrocchi A, Hoxha A, Simonsen G S, et al. Attributable deaths and disability-adjusted life-years caused by infections with antibiotic-resistant bacteria in the EU and the European Economic Area in 2015: a population-level modelling analysis. The Lancet Infectious Diseases 2019; 19:56-66.) MDR E. coli is currently one of the largest clinical burdens facing both human and animal health.

Escherichia coli is a Gram-negative, rod-shaped bacterium, member of the family Enterobacteriaceae, genus Escherichia. It is the most prevalent commensal bacterial member of the gastrointestinal tract of warm-blooded animals as well as a very prevalent pathogen, responsible for a broad spectrum of diseases. According to the virulence factors present in the pathogenic strains and the clinical symptoms of the host, E. coli isolates can be classified in distinct pathogenic types (or pathotypes, which are defined as a group of strains of the same species causing a common disease). (Allocati N, Masulli M, Alexeyev M F, Di Ilio C, et al. Escherichia coli in Europe: An Overview. International Journal of Environmental Research and Public Health. 2013; 10 (12): 6235-6254.) One of such pathotypes is Avian Pathogenic Escherichia coli (APEC), an extra-intestinal pathogenic E. coli (ExPEC) which causes diverse local and systemic infections in avian species (including chicken, turkeys, ducks, among many others). The most common infections caused by APEC are perihepatitis, airsacculitis, pericarditis, egg peritonitis, omphalitis, cellulitis, coligranuloma and osteomyelitis/arthritis (all of these are commonly referred as colibacillosis). (Kathayat D, Lokesh D, Ranjit S, Rajashekara G, et al. Avian Pathogenic Escherichia coli (APEC): An Overview of Virulence and Pathogenesis Factors, Zoonotic Potential, and Control Strategies. Pathogens. 2021; 10(4): 467.) Colibacillosis is one of the main causes of mortality in poultry (up to 20%), and also causes a decrease in meat and egg production. Moreover, APEC is responsible for up to 35% of deaths in young chickens. Taking everything together, in the United States only, it is estimated that APEC causes economic losses of up to $40 million annually to the broiler industry (only due to carcass condemnation) (Id.)

Therefore, there is an urgent need for alternatives to antibiotics not only for treating pathogenic E. coli associated infections but also for sanitizing contaminated environments.

### INCORPORATION BY REFERENCE

All scientific publications and patent applications identified herein are incorporated by reference in their entirety and to the same extend as if each individual publication or patent application was specifically and individually indicated to be incorporated by reference.

Isolated bacteriophage FP1.1 was deposited as NCIMB Accession No. 44022 on Aug. 30, 2022, isolated bacteriophage FP1.2 was deposited as NCIMB Accession No. 44023 on Aug. 30, 2022, and bacteria E. coli A1.MCA.1 was deposited as NCIMB Accession No. 44024 on Aug. 30, 2022 with the British National Collection of Industrial, Food and Marine Bacteria (NCIMB), Ferguson Building, Craibstone Estate, Bucksbum, Aberdeen AB21 9YA, UK. These deposits were made under the provisions of the Budapest Treaty on the International Recognition of the Deposit of Microorganisms for the Purposes of Patent Procedure and the Regulations thereunder.

### SUMMARY

This disclosure describes biocontrol compositions including at least one isolated bacteriophage, as well as methods of producing and using such biocontrol compositions.

The invention concerns two jumbo bacteriophages of the order Caudovirales, referred to herein as FP1.1 and FP1.2, having a broad lytic activity against multiple E. coli strains, genotypes and pathotypes (See, FIGS. 1a, 1b, 9 and 10). FP1.1 is a new species belonging to the viral genus Asteriusvirus, and FP1.2 is a new species belonging to the viral genus Goslarvirus.

The invention further concerns an isolated nucleic acid sequence of the genome of said bacteriophage selected from the SEQ ID NO: 3 through SEQ ID NO: 964 (see Tables 2 and 3), and gene products encoded by a nucleic acid sequence selected from the SEQ ID NO: 3 through SEQ ID NO: 964 (see Tables 2 and 3), or one catalytic/functional domain derived from the gene product.

The invention includes a composition including at least one of the phages described herein, at least one phage-derived protein/polypeptide (such as endolysin) involved in the lytic cycle or any phage-derived protein(s) with excipients.

The invention relates to at least one bacteriophage selected from FP1.1 and FP1.2 as described herein or their combination in any ratio or formulation, having substantial lytic activity against at least one pathogenic E. coli strain, wherein such bacteriophage or cocktail is active against at least one Avian Pathogenic E. coli (APEC) selected from the list provided in FIG. 1a, other avian pathogenic E. coli variants, and human pathogenic E. coli strains that exhibit multi drug resistance (MDR), specifically Extended-Spectrum Beta-Lactamases (ESBL) strains as listed in FIG. 9.

The invention includes a method for propagation of the bacteriophages described herein together, and co-propagation in the same bacterial host at the same time. Such propagation is performed in a non-pathogenic E. coli strain to ensure that the biocontrol composition is safe to be used for treatment.

The invention also relates to the use of any combination of bacteriophages, their nucleic acids or their products as described herein, for treating a human infected by at least one E. coli bacteria selected from the strains listed in FIG. 9.

The invention also relates to a method for reducing the E. coli load in poultry production farms, by applying a composition of bacteriophages, and/or their nucleic acids or gene products as described herein, for reducing the load of the bacterium in the birds themselves, decontaminating surfaces, equipment, water and feed.

The invention also relates to a method for treating an avian patient infected or susceptible of being infected with at least one APEC and other E. coli pathotypes such as the bacteria selected from the lists provided in FIGS. 1a and 10, by administering an effective amount of the composition of bacteriophages, and/or their nucleic acids or gene products as described herein.

The invention also relates to the use of any composition of bacteriophages, and/or their nucleic acids or gene products for the bioremediation of environments contaminated with E. coli preferably contaminated or at risk of being contaminated with at least one bacterial strain listed in FIG. 1b such as small bodies of water, sewage, slaughterhouses, hospital environments and medical equipment, soil, among others.

The invention may be used for treating humans, other mammals or avian species, or for sanitizing any material, equipment or contaminated environment.

Further aspects of the present disclosure are provided by the subject matter of the following clauses.

A biocontrol composition including at least one distinct bacteriophage having substantial lytic activity against at least one pathogenic bacterial strain, the at least one distinct bacteriophage selected from the group consisting of a bacteriophage having (i). a genome having an overall sequence identity with at least 70% of the genome of the bacteriophage deposited under Accession No. NCIMB44022; (ii). a genome that includes the nucleotide sequence of SEQ ID NO: 1, or a genome including at least 70% overall sequence identity with the nucleotide sequence of SEQ ID NO: 1; (iii). a genome including an overall sequence identity with at least 70% of the genome of the bacteriophage deposited under Accession No. NCIMB44023; and (iv). a genome including the nucleotide sequence of SEQ ID NO: 2, or a genome including at least 70% overall sequence identity with the nucleotide sequence of SEQ ID NO: 2, and a pharmaceutically, veterinary or environmentally acceptable excipient or carrier including a preservative in an amount effective to preserve the substantial lytic activity of at least one bacteriophage.

A biocontrol composition including a combination of the at least two distinct bacteriophages, having substantial lytic activity against at least one pathogenic bacterial strain, wherein (i). the first bacteriophage has a genome including an overall sequence identity with at least 70% (for example, at least 75%, at least, 80%, a least 85%, at least 90%, at least 91%, at least 92%, at least 93%, at least 94%, at least 95%, at least 96%, at least 97%, at least 98%, or at least 99%) of the genome of the bacteriophage deposited under Accession No. NCIMB44022, or a genome including the nucleotide sequence of SEQ ID NO: 1, or a genome including at least 70% (for example, at least 75%, at least, 80%, a least 85%, at least 90%, at least 91%, at least 92%, at least 93%, at least 94%, at least 95%, at least 96%, at least 97%, at least 98%, or at least 99%) overall sequence identity with the nucleotide sequence of SEQ ID NO: 1; and (ii). the second bacteriophage has a genome including an overall sequence identity with at least 70% (for example, at least 75%, at least, 80%, a least 85%, at least 90%, at least 91%, at least 92%, at least 93%, at least 94%, at least 95%, at least 96%, at least 97%, at least 98%, or at least 99%) of the genome of the bacteriophage deposited under Accession No. NCIMB44023, or a genome including the nucleotide sequence of SEQ ID NO: 2, or a genome including at least 70% (for example, at least 75%, at least, 80%, a least 85%, at least 90%, at least 91%, at least 92%, at least 93%, at least 94%, at least 95%, at least 96%, at least 97%, at least 98%, or at least 99%) overall sequence identity with the nucleotide sequence of SEQ ID NO: 2.

A method of controlling at least one pathogenic bacterial strain by exposing said at least one pathogenic bacterial strain to a biocontrol composition including a mixture of at least two distinct bacteriophages, wherein (i). a first bacteriophage has a genome having an overall sequence identity with at least 70% (for example, at least 75%, at least, 80%, a least 85%, at least 90%, at least 91%, at least 92%, at least 93%, at least 94%, at least 95%, at least 96%, at least 97%, at least 98%, or at least 99% sequence identity) of the genome of the bacteriophage deposited under Accession No. NCIMB44022, or a genome comprising the nucleotide sequence of SEQ ID NO: 1, or a genome comprising at least 70% (for example, at least 75%, at least, 80%, a least 85%, at least 90%, at least 91%, at least 92%, at least 93%, at least 94%, at least 95%, at least 96%, at least 97%, at least 98%, or at least 99%) overall sequence identity with the nucleotide sequence of SEQ ID NO: 1; and (ii). a second bacteriophage has a genome comprising an overall sequence identity with at least 70% (for example, at least 75%, at least, 80%, a least 85%, at least 90%, at least 91%, at least 92%, at least 93%, at least 94%, at least 95%, at least 96%, at least 97%, at least 98%, or at least 99% sequence identity) of the genome of the bacteriophage deposited under Accession No. NCIMB44023, or a genome comprising the nucleotide sequence of SEQ ID NO: 2, or a genome comprising at least 70% (for example, at least 75%, at least, 80%, a least 85%, at least 90%, at least 91%, at least 92%, at least 93%, at least 94%, at least 95%, at least 96%, at least 97%, at least 98%, or at least 99%) overall sequence identity with the nucleotide sequence of SEQ ID NO: 2.

An embodiment of the invention relates to a method of producing a biocontrol composition having substantial lytic activity against a broad range of pathogenic bacterial strains including the steps of
(a). Maintaining a single non-pathogenic bacteria strain in a vessel
(b). Seeding said single bacterial strain culture with a co-culture of
   (i). a distinct bacteriophage having a genome having an overall sequence identity with at least 70% (for example, at least 75%, at least, 80%, a least 85%, at least 90%, at least 91%, at least 92%, at least 93%, at least 94%, at least 95%, at least 96%, at least 97%, at least 98%, or at least 99%) of the genome of the bacteriophage deposited under Accession No. NCIMB44022, or having a genome including the nucleotide sequence of SEQ ID NO: 1, or a genome including at least 70% (for example, at least 75%, at least, 80%, a least 85%, at least 90%, at least 91%, at least 92%, at least 93%, at least 94%, at least 95%, at least 96%, at least 97%, at least 98%, or at least 99%) overall sequence identity with the nucleotide sequence of SEQ ID NO: 1, and
   (ii). a distinct bacteriophage having a genome having an overall sequence identity with at least 70% (for example, at least 75%, at least, 80%, a least 85%, at least 90%, at least 91%, at least 92%, at least 93%, at least 94%, at least 95%, at least 96%, at least 97%, at least 98%, or at least 99%) of the genome of the bacteriophage deposited under Accession No. NCIMB44023, or having a genome including the nucleotide sequence of SEQ ID NO: 2, or a genome including at least 70% (for example, at least 75%, at least, 80%, a least 85%, at least 90%, at least 91%, at least 92%, at least 93%, at least 94%, at least 95%, at least 96%, at least 97%, at least 98%, or at least 99%) overall sequence identity with the nucleotide sequence of SEQ ID NO: 2,
(c). Harvesting the bacteriophages from the bacterial culture.

### BRIEF DESCRIPTION OF THE DRAWINGS

FIGS. 1a and 1b show tables of the host-range of FP1 cocktail, FP1.1 and FP1.2 across all strains tested isolated from poultry and sewage/different environments, respectively.

FIG. 2 is a bar graph showing the number of bacteria isolates tested according to their origin, human, poultry and sewage.

FIG. 3 is a bar graph comparing the host range of FP1.1, FP1.2 and FP1 (cocktail of co-propagated FP1.1 and FP1.2) across all tested strains

FIG. 4 is a bar graph comparing the host range of FP1 cocktail across three different groups of strains isolated from Human, Sewage, and Poultry

FIG. 5 is a bar graph showing the differences in host-range scores of FP1.1, FP1.2, artificial (FP1.A, FP1.B, and FP1.C) and FP1.

FIG. 6 is a graph comparing the fold change in virulence increase per strain of FP1 when compared with artificial cocktails.

FIG. 7 is a graph comparing the difference in overall virulence of the FP1 when compared with the artificial cocktail across all strains.

FIG. 8A-B shows transmission electron micrographs of bacteriophages FP1.1 and FP1.2, respectively.

FIG. 9 shows a table of the host-range of FP1, FP1.1 and FP1.2 across human pathogenic E. coli strains.

FIG. 10 shows a table of host-range of FP1, FP1.1 and FP1.2 across APEC strains.

FIG. 11 shows the results of the treatment of a coliform contaminated creek with FP1 cocktail; results of the coliform test in MacConkey agar of the non-treated (A) and FP1-treated (B) conditions; coliform-like colonies can be seen as dark spots in the plates; (C) shows quantitative results of CFU/100 ml of coliform-like colonies in the non-treated vs treated conditions.

FIG. 12 shows a table with the results of the pH resistance test for FP1.1 and FP1.2.

### DETAILED DESCRIPTION

Some embodiments of the current invention are discussed in detail below and in the accompanying drawings. In describing embodiments, specific terminology is employed for the sake of clarity. However, the invention is not intended to be limited to the specific terminology so selected. The invention is also not intended to be limited to the embodiment depicted by the drawings. A person skilled in the relevant art will recognize that other equivalent components can be employed and other methods developed without departing from the broad concepts of the current invention. All references cited anywhere in this specification, including the Background and Detailed Description sections, are incorporated by reference as if each had been individually incorporated.

Definitions are included herein for the purpose of understanding the present subject matter and the appended patent claims and drawings. The abbreviations used herein have their conventional meanings within the chemical and biological arts.

While various embodiments and aspects of the present invention are shown and described herein, it will be obvious to those skilled in the art that such embodiments and aspects are provided by way of example only. Numerous variations, changes, and substitutions will occur to those skilled in the art without departing from the invention. It should be understood that various alternatives to the embodiments of the invention described herein may be employed in practicing the invention.

Unless defined otherwise, technical and scientific terms used herein have the same meaning as commonly understood by a person of ordinary skill in the art. Any methods, devices and materials similar or equivalent to those described herein can be used in the practice of this invention. The following definitions are provided to facilitate understanding of certain terms used frequently herein and are not meant to limit the scope of the present disclosure.

The present description identifies certain nucleotide sequences as part of the invention. It is to be understood that the specifically identified sequences adequately describe other sequences that contain less than 100% sequence identity but identified sequences that provide substantially the same phenotype or function. A nucleotide sequence may have at least 70% at least 75%, at least, 80%, a least 85%, at least 90%, at least 91%, at least 92%, at least 93%, at least 94%, at least 95%, at least 96%, at least 97%, at least 98%, or at least 99% sequence identity with a nucleotide sequence specifically disclosed herein and still encodes for an entirely equivalent or functionally equivalent polypeptide. In another example, a variant may have a nucleotide sequence having at least 70% at least 75%, at least, 80%, a least 85%, at least 90%, at least 91%, at least 92%, at least 93%, at least 94%, at least 95%, at least 96%, at least 97%, at least 98%, or at least 99% sequence identity with the nucleotide sequence of one of the phage genomes specifically disclosed herein; such variant has at least one variation in its genomic sequence as compared to the genomic sequence disclosed herein while retaining at least one phenotypic characteristic of the phage, The preserved phenotypic characteristic of the phage can be, for example, the ability to transduce their genome, replicate and propagate, kill the same host range or adapt to new hosts that are members of the same family as their original hosts. Preferably, variants retain at least their ability to transduce their genome into the same hosts or closely related hosts. The phenotypic characteristic of the phage can also be defined by its morphology, its biochemistry including its ability to resist certain conditions, such as extreme pH, and/or its host-range behavior against certain bacteria strains including its virulence, and its ability to bind, replicate in, and lyse at least one bacterial strain. Variants may arise naturally or be engineered using well known methods in the art. As used herein, recombinant phages may derive from the naturally occurring phages of the invention or their variants whose genome incorporates at least one heterologous nucleic acid.

As used throughout, the term “bacteriophage” or “phage” refers to a virus with the capacity of infecting exclusively bacteria and composed of a nucleic acid packaged inside a protein envelope or capsid. It includes the parent bacteriophage, the progeny and any derivatives including variants that may occurred naturally or have been genetically engineered or recombinant phages thereof.

Throughout, the term “lytic activity” refers to the ability of a bacteriophage to kill a bacterium by causing the lysis of the bacterial cell. Standard, state of the art methods for assessing this ability can be seen in the experimental section.

The term “APEC” which stands for “Avian Pathogenic Escherichia coli” refers to an extra-intestinal pathogenic E. coli that commonly causes colibacillosis disease in avian species.

The term “ESBL E. coli” refers to Extended-Spectrum Beta-Lactamases-producing E. coli, an antibiotic resistant strain with specific resistance to oxy-imino cephalosporins and monobactams.

The term “multi-resistance” or “multidrug resistance” or “MDR” in bacteria refers to the accumulation of antibiotic resistance in a bacterium against at least one antibiotic, including but not limited to ampicillin, cefazolin, ceftazidime, ceftriaxone, cefepime, ciprofloxacin, trimethoprim, sulfamethoxazole, piperacillin, tazobactam, beta-lactam, aminoglycoside, macrolide Preferably, MDR refers to antibiotic resistance to at least three of the antibiotic classes.

The term “phage phenotype” or “phenotypic characteristic” can be defined by its morphology, its biochemistry including its ability to resist certain conditions, such as extreme pH, and/or its host-range behavior against certain bacteria strains including its virulence, and its ability to bind, replicate in, and lyse at least one bacterial strain. It can also refer to an infective characteristic of the bacteriophage, growth pattern and host-range. Methods are available for qualitatively and/or quantitatively assess such characteristics against specific bacteria.

The term “host-range” refers to the extent of the lytic activity of a bacteriophage against a group of bacteria. A “broad host-range” refers to the ability of the phage to kill a diversity of strains targeted, the diversity being defined in terms of origin and/or genotype and/or virulence. Accordingly, the compositions of the invention display a broad range-host as they can kill more than 60% of the 113 diverse strains tested. Such bacteria may be E. coli, other Escherichia species and other related bacterial species.

The term “biocontrol” refers to the reduction of the load of bacteria in a specific setting to a point where their presence poses no harm or that allows infection to solve themselves.

The term “% of identity” related to a comparison between nucleic acid sequences designates the degree of similarity or homology between said sequences. This homology can be determined by comparing the sequences using bioinformatic software.

The term “specificity” of a bacteriophage refers to the type of host that it is able to infect. For example, a phage “specific” for E. coli can infect multiple E. coli isolates and cannot infect other non-E. coli isolates.

The term “co-propagate” or “co-propagation” refers to the ability of two or more different phages to infect the same bacterium and replicate in the same bacterium simultaneously. Notably, the phages of the invention have the ability to infect and replicate in the same bacterium simultaneously. They can also infect and replicate in bacteria independently. Notably, the bacteriophages FP1.1 and FP1.2 according to the invention tolerate each other in the same bacterium and can cooperate to enhance or potentiate their lytic activity against certain bacterial strains.

The term “isolated bacteriophages” refers to bacteriophages that have been separated from their natural environment, specifically, a phage or phages that have been cultured and purified separated from the environment in which they were present originally.

The term “PFU” refers to Plaque Forming Unit, which measure the quantity of bacteriophages that are capable of lysing bacteria and forming a clearing zone or plaque around the infected bacteria.

The term “mammal” includes humans, livestock such as cows, pigs, horses, ruminants, sheep, goats, etc., as well as pets (e.g., dogs, cats).

The term “avian species” refers to any avian species used as livestock for human consumption e.g., poultry, turkeys, geese.

The term “veterinary, pharmaceutically or environmentally acceptable” for a carrier or excipient refers to any material that complies with international standards for that use, mainly, solutions that are demonstrated to pose no harms to the organism and/or does not elicit any undesirable immune reactions while retaining the efficacy and stability of the composition and preserving the substantial lytic activity of the bacteriophages. For liquid formulation, the biocontrol composition may comprise saline, sterile water, Ringer's solution, buffered physiological saline, albumin infusion solution, dextrose solution, maltodextrin solution, glycerol, ethanol, and mixtures thereof may be used as a pharmaceutically or veterinary acceptable excipient or carrier. If require, other conventional additives such as thickeners, diluents, buffers, preservatives, binders, dispersants, surface active agents, antioxidants and bacteriostatic agents may be added to the biocontrol composition.

The term “treatment”, “treat” or “therapy” refers to prophylactic or preventive treatment as well as curative treatment, including treatment of subjects at risk of contracting the bacterial infection or suspected to have contracted a bacterial infection. Alternatively, the term “treatment” or “treat” can be also applied to the decontamination of an environment or equipment that have been exposed or at risk of exposure to a bacterial contamination.

The term “virulence” when applied to a bacteriophage composition refers to a degree of lytic (causing or resulting from lysis) activity at a given condition. Virulence may indicate the ability of a phage to undertake lytic rather than lysogenic cycles. A lytic or virulent phage is able to self-replicate and has high specificity against its bacterial host. Virulence may indicate the potential of a phage to drive target bacterial cultures to extinction or, at least, to low densities. Virulence may also be defined as the ability of a phage to control the growth of the target bacteria in culture, i.e., culture clearing or may be an indicator of phage utility. Sometimes virulence is linked to the phage's burst size as a prerequisite for productive-infection treatment.

The term “susceptible” when applied to the effect of a bacteriophage or a biocontrol composition on a bacterial strain, refers to the substantial lytic activity of the bacteriophage in the composition that show complete kill or at least 70%, 80%, preferably at least 85%, and most preferably at least 90% or more in reduction of the number of bacterial cells in a bacterial lawn after bacteriophage treatment as compared to the number of bacterial cells before treatment. In the context of phage therapy, treated subjects such as humans, other mammals or avian should show a significant clinical recovery after treatment.

The term “semi-susceptible” when applied to the effect of a bacteriophage or a biocontrol composition on a bacterial strain, refers to the lytic activity of the bacteriophages in the preparation that exhibits incomplete kill of the bacteria, or less than 70% but more than 25% in reduction of the number of bacterial cells in a bacterial lawn after bacteriophage treatment as compared to the number of bacterial cells before treatment.

The term “substantial lytic activity” refers to the lytic activity of the bacteriophages or the biocontrol composition on susceptible and semi-susceptible bacterial strains.

The term “resistant” when applied to the effect of a bacteriophage composition on a bacterial strain, refers to the lytic activity of the bacteriophages in the preparation that exhibits less than 25% kill or no kill of the number of bacterial cells in a bacterial lawn after bacteriophage treatment as compared to the number of bacterial cells before treatment.

The term “jumbo phages” refers to tailed bacteriophages whose genome is longer than 200 kb; they usually are characterized by their large capsids. The term highlights how unusual this genome length is for these kinds of viruses. Jumbo phages do not compose a taxonomic classification according to the ICTV but spread across various families in the Caudovirales order.

The present invention is related to novel bacteriophage-based biocontrol compositions. Specifically, the present invention relates to novel bacteriophages having substantial lytic activity and broad host-range against Escherichia coli strains, their manufacture, components thereof, biocontrol compositions including the same, and their uses.

The inventors have isolated and characterized two novel bacteriophages that combined a broad lytic activity against pathogenic bacteria including E. coli, which can be used as active biocontrol agents in pharmaceutical, veterinary and decontamination compositions. Specifically, the bacteriophage composition can be used in the treatment and/or prophylaxis of E. coli related infections in humans or animals. The phage compositions can significantly control and reduce the load of E. coli in livestock, production farms, and desirable environments. Importantly, the new bacteriophages of the invention present substantial lytic activity against a broad spectrum of bacterial strains such as strains of E. coli, high endurance to harsh environmental conditions, and high stability for long periods of time.

### Characterization of the Bacteriophages

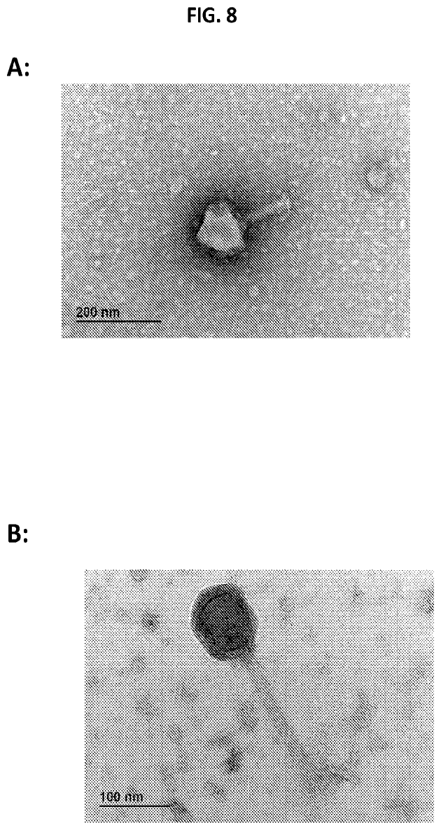

In this invention, two novel bacteriophages against E. coli, designated herein as FP1.1 and FP1.2 were isolated from the same environmental sample and characterized. They are double stranded DNA (dsDNA) tailed phages belonging to the Caudovirales order. (See, FIGS. 8A and 8B). FP1.1 is a new species belonging to the viral genus Asteriusvirus, and FP1.2 is a new species belonging to the viral genus Goslarvirus. Because of their large genome size (>200 kb), FP1.1 and FP1.2 are both classified as jumbo phages. Bacteriophages FP1.1 and FP1.2 have a genome having the nucleotide sequence SEQ ID NO:1 and SEQ ID NO:2, respectively.

TABLE 1

| SEQ ID | Bacteriophage |
|---|---|
| SEQ ID NO: 1 | FP1.1 |
| SEQ ID NO: 2 | FP1.2 |

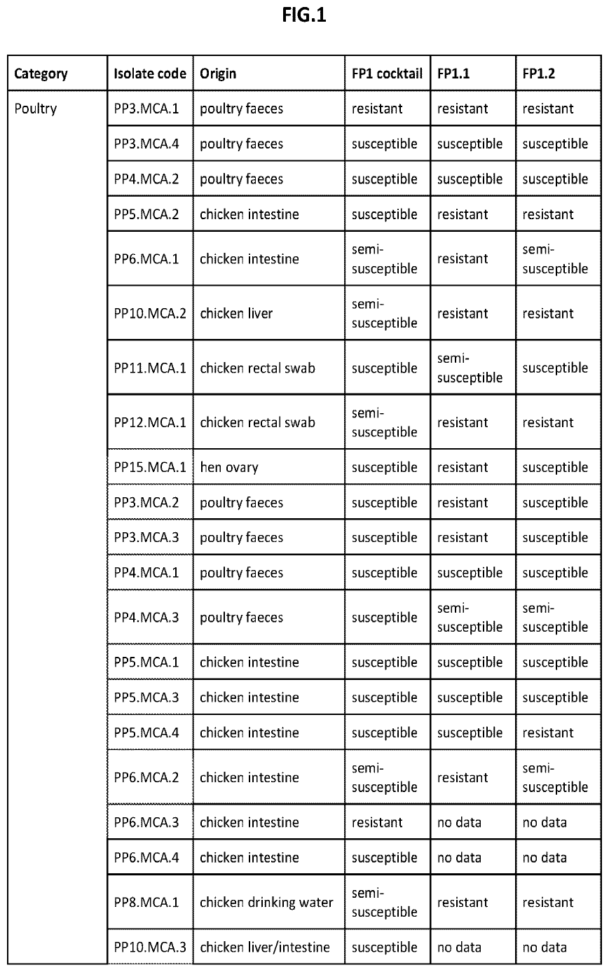

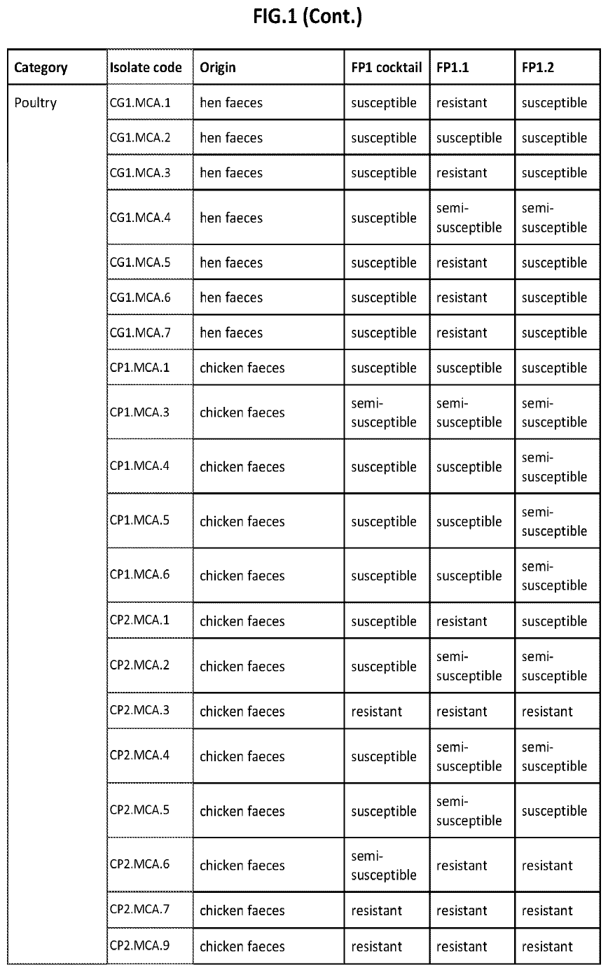

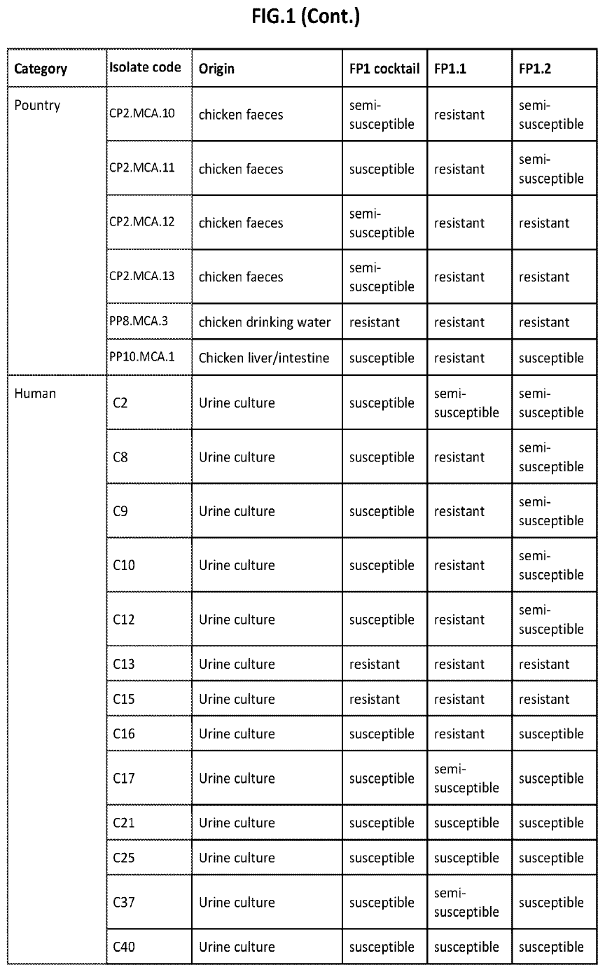

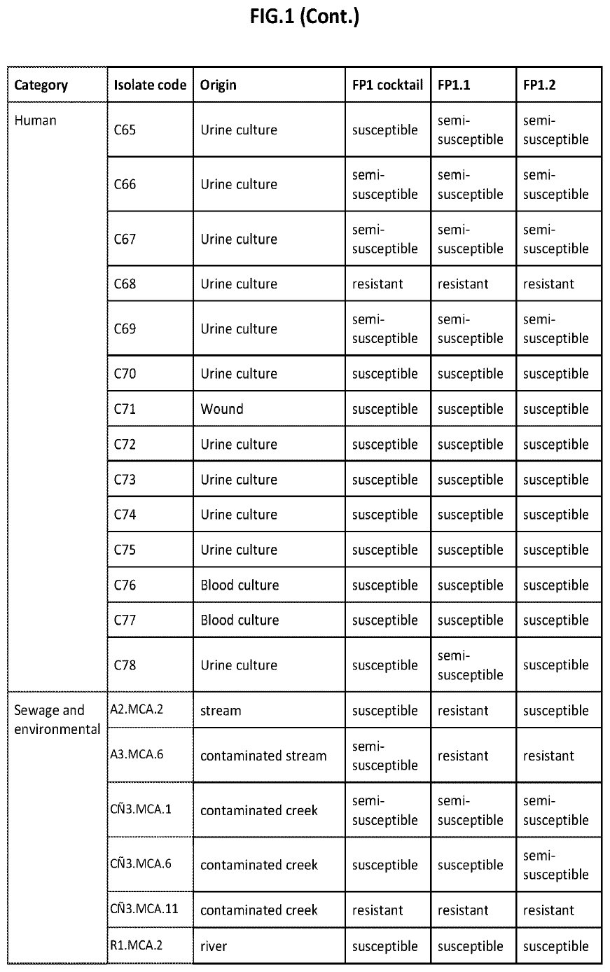

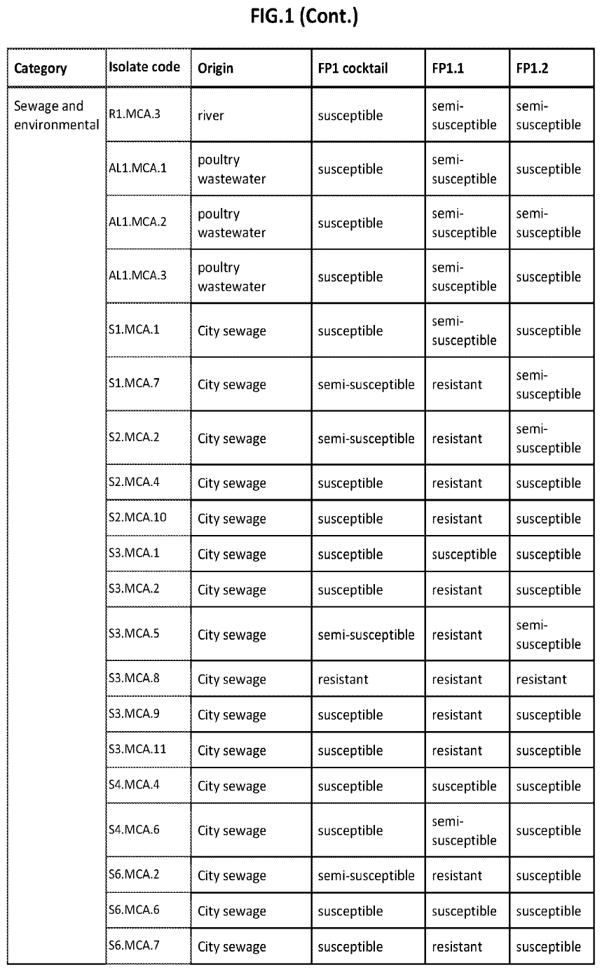

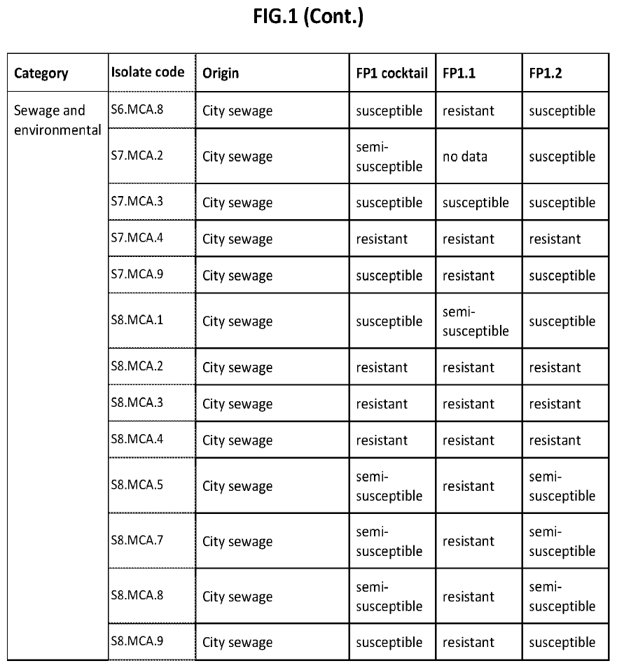

Unlike known bacteriophages which usually display narrow host range, the two bacteriophages of the invention, either together or individually have a remarkable broad host-range against diverse E. coli strains isolated from different sources. (See, FIGS. 1a, 1b, 2 and 9). Each bacteriophage FP1.1 and FP1.2 is active against a multitude of E. coli strains, other Escherichia species and to a lesser extent other related species such as Salmonella enterica and Citrobacter freundii. However, they do not have substantial lytic effect against Enterobacter cloacae, Enterobacter asburiae, Proteus mirabilis, Klebsiella pneumoniae, Klebsiella oxytoca, Klebsiella variicola, and Raoultella ornithinolytica.

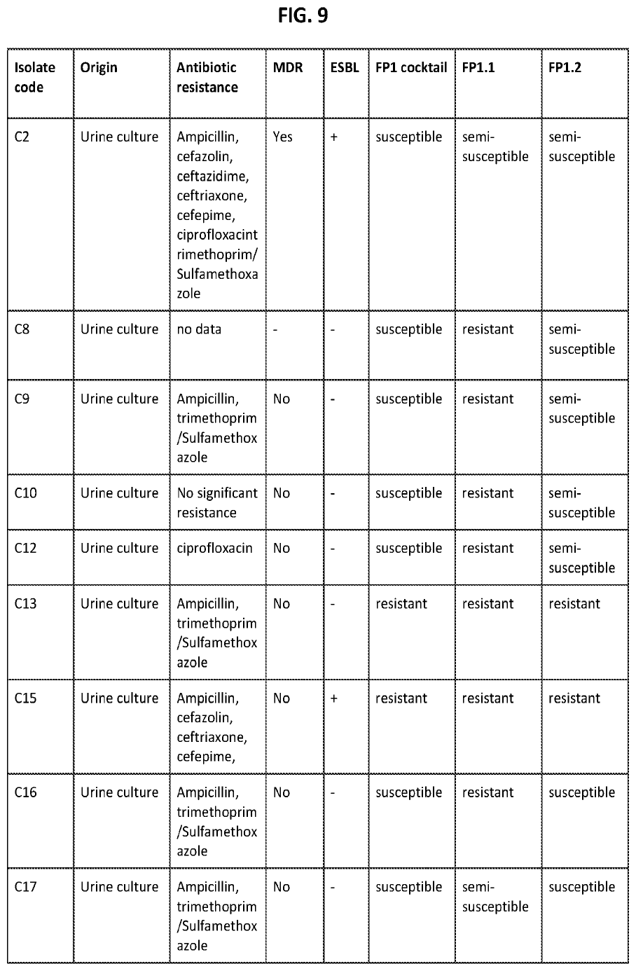

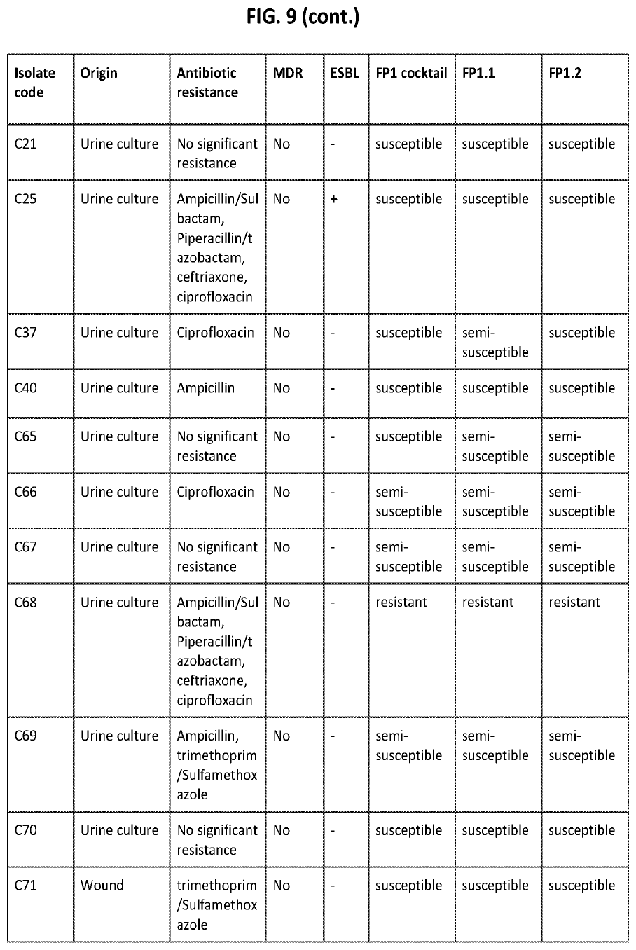

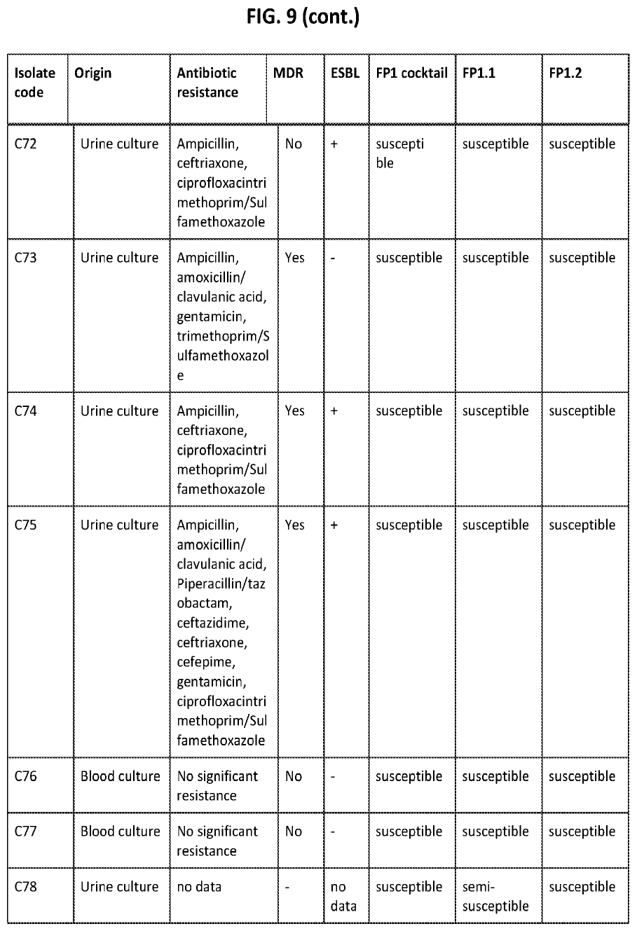

The activity and the phenotypic characteristics of the bacteriophages described in the present invention can be assessed by methods well-known in the art, like spot testing, based on growing bacterial lawns with the spot of a phage preparation on top. In this method, the bacteriophage induces the lysis of target E. coli cells after a period of incubation at an appropriate temperature for bacterial growth, and as a result, a clearing where in the place of the spot test is formed. The bacteriophages of the invention, when combined, exhibit a remarkable lytic activity against pathogenic E. coli strains isolated from human samples (urine and blood samples), specifically multi-drug resistant and ESBL strains (See, FIG. 9), and from pathogenic E. coli strains isolated from poultry farms, specifically APEC strains. (See, FIG. 1a)

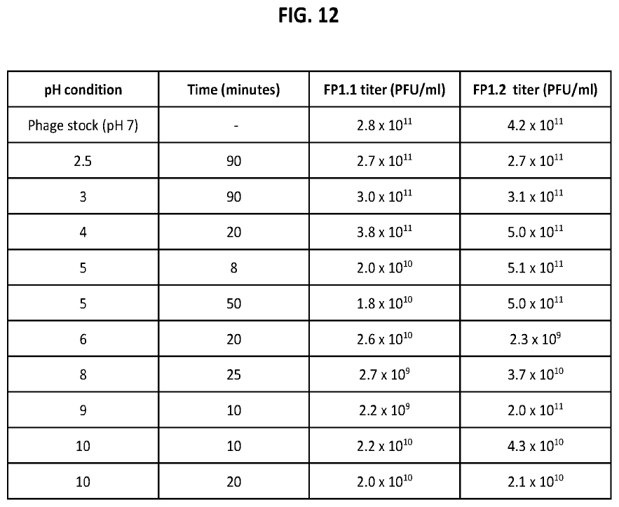

The bacteriophages of the invention remain stable and retain their substantial lytic activity in different environmental conditions; for example: at low and ambient temperatures ranging from 4 °C to 42 °C, such as 10 °C, 24 °C, 37 °C and 42 °C, and at throughout a broad pH range, for example 2.5 to 11, thus making them suitable for all applications stated in the invention. (See, FIG. 12)

The bacteriophages of the invention include FP1.1 and FP1.2 as well as their derivatives, including variants and recombinants of FP1.1 and FP1.2 as exemplified in the section below.

### Nucleic Acids

In one aspect, the invention comprises any nucleic acid variants, fragments or genes derived from the FP1.1 and FP1.2 genomes having the nucleotide sequence SEQ ID NO: 1 or SEQ ID NO:2, respectively.

Phage variants of FP1.1 or FP1.2 may be defined as having homologous sequences to wild type FP1.1 and FP1.2, including but not limited to, naturally or artificially modified variants using any of the methods well-known in the art. For example, sequences in which nucleic acid insertion, deletions, substitution, inversion and rearrangements occur. FP1.1 and FP1.2 variants having homologous sequences may retain at least one phenotypic characteristic of wild-type FP1.1 or FP1.2 such as the ability to transduce their genome, propagate and lyse the same host range and/or adapt to new hosts that are members of the same family as their original hosts.

In some embodiments, FP1.1 and FP1.2 phages may be modified by deletion and/or mutations in genes that are non-essential to their lytic cycle. Homologous recombination is a common technique to delete or exchange target nucleic acid sequences in temperate phages. Other techniques based on CRISPR-Cas systems also allow accurate deletions of genes which can subsequently be replaced by desirable nucleic acid sequences. Phenotype of such FP1.1 or FP1.2 variants may be screened directly after plating. For instance, deletion and/or mutations in lytic genes may be validated by a “clear lysis plaque” phenotype easily identified by the skilled person in the art.

In another aspect of the invention, the genome of FP1.1, FP1.2 or their variants may be further modified to incorporate at least one heterologous nucleic acid that enhances the killing of their host, defined herein as recombinant phages FP1.1 and FP1.2. The heterologous nucleic acid may encode proteins or RNA sequences that target specific genes within the bacterial genome that when targeted, induce lysis. The heterologous nucleic acid may encode proteins or RNA sequences that interfere or inactivate the immune system CRISPR-Cas of the host (Acr). The heterologous nucleic acid sequence may also encode proteins and/or RNA sequences that induce bacterial DNA degradation, and therefore, death. Such nucleic acid may encode one or several components of the CRISPR-Cas system targeting the host genome (Cas protein, specific crRNA/spacer, cascade proteins, with or without transcription activator). The proteins and/or RNA sequences encoded by the heterologous nucleic acid may also target specific bacterial genes that determine pathogenicity (such as toxins) and render them ineffective, therefore, making the bacteria innocuous. The heterologous nucleic acid sequence may also modify the bacteriophage's host-range, efficiency of entry into bacterial cells, efficiency of replication, and enhance lysis (for example, by containing a potent endolysin derived from another bacteriophage).

In another aspect of the invention, the genome of FP1.1, FP1.2 or their variants may be further engineered with nucleic acid segments deleted from their genomes to incorporate heterologous nucleic acids, as stated herein, and/or to remove unwanted functions/activity and/or to simply shorten the bacteriophage genome to a minimal, yet functional size.

In another aspect, the invention relates to any nucleic acid within the phage genomes that encodes for a functional polypeptide/protein or a catalytic and/or functional domain thereof. Such polypeptides/proteins may include, but are not limited to polypeptides/proteins involved in any of the stages of the lytic cycle e.g., the attachment of the phage to the surface of the host cell, the transduction of its genome, its replication, the biosynthesis and maturation of new phage particles and the lysis of the host cell, or polypeptides/proteins that provide a competitive survival advantage to the bacteriophages of the invention over other bacteriophages, or polypeptides/proteins involved in mechanism(s) that favor the co-propagation of the bacteriophages of the invention within the same bacterium. The nucleotide sequences of SEQ ID NO: 3 to SEQ ID NO: 964 encode proteins/polypeptides of the genome of FP1.1 and FP1.2 (listed in Tables 2 and 3, respectively) and may be used in the biocontrol compositions of the invention. Similarly, proteins/polypeptides encoded by the nucleotide sequences of SEQ ID NO: 3 to SEQ ID NO: 964 may be used in the biocontrol compositions according to the invention.

In addition, the invention is also related to a composition that includes at least one nucleic acid sequence according to at least one of SEQ ID NO: 3 to SEQ ID NO: 964, or at least one protein/polypeptide encoded by at least one of SEQ ID NO: 3 to SEQ ID NO: 964. The biocontrol composition may include the at least one nucleic acid sequence inserted in an expression cassette to facilitate its expression in the appropriate host.

The invention is also related to a composition that includes at least one nucleic acid sequence which encodes for at least one of the components of a CRSIPR-Cas system. Such composition may be used for in vivo and in vitro gene editing, including but not limited to point mutations, frameshift mutations, fragment insertions, gene deletions, and gene replacements. In another aspect of the invention, the composition includes at least one nucleic acid sequence which encodes for a functional DNA polymerase involved in DNA replication, DNA repair or genetic recombination. Such composition may be used in vitro as a tool in genetic engineering.

The invention is also related to a composition as an effective and safe phage-based nanocarrier system capable of delivering a therapeutic agent such as a nucleic-based therapeutic or a drug in host cells. In one aspect, one of the bacteriophages of the invention may be modified to enhance the delivery and/or targeting of the nucleic-based therapeutic such as a transgene expression cassette or a drug into mammalian cells, for example by chemically modifying the capsid of the bacteriophage with a cationic polymer. The capsid may be further modified to display a targeting ligand to specifically deliver the nucleic-based therapeutic or drug into the targeted cells. An example of targeting ligand may be a membrane expressing tumor marker. The bacteriophage of the invention may be used as a phage-based nanocarrier for the delivery of antibiotics to enhance the bactericide effect.

The invention is also related to a composition containing at least one protein/polypeptide encoded by at least one of SEQ ID NO: 3 to SEQ ID NO: 964 as an effective and safe system for delivering a therapeutic agent in host cells, and methods of using the composition. The therapeutic agent can include, but is not limited to, a nucleic-based therapeutic or a drug. In any aspect of this embodiment, the at least one bacteriophage-derived protein, alone or in combination with other proteins, can assemble into a nanostructure to deliver the therapeutic agent. Non-limiting examples of proteins capable of assembling into nanostructures may be tubulin PhuZ (SEQ ID NO: 874) and nuclear shell protein (SEQ ID NO: 889) derived from bacteriophage FP1.2. In any aspect of this embodiment, the host cell is a eukaryotic cell.

TABLE 2
Protein-encoding genes from the genome of the bacteriophage FP1.1.

| SEQ | | | Gene | | | |
|---|---|---|---|---|---|---|
| ID NO | Gene id | Annotation | length | Start | End | Strand |
| 3 | FP1.1_CDS_0087 | putative protein | 642 | 1 | 642 | − |
| 4 | FP1.1_CDS_0088 | putative protein | 846 | 623 | 1468 | − |
| 5 | FP1.1_CDS_0089 | putative protein | 498 | 1473 | 1970 | − |
| 6 | FP1.1_CDS_0090 | putative protein | 429 | 1980 | 2408 | − |
| 7 | FP1.1_CDS_0091 | DNA helicase | 1485 | 2464 | 3948 | − |
| 8 | FP1.1_CDS_0092 | DNA polymerase exonuclease | 819 | 3964 | 4782 | − |
| | | subunit | | | | |
| 9 | FP1.1_CDS_0093 | putative protein | 462 | 4830 | 5291 | − |
| 10 | FP1.1_CDS_0094 | UvsX-like recombinase | 1119 | 5291 | 6409 | − |
| 11 | FP1.1_CDS_0095 | single strand DNA binding | 1053 | 6479 | 7531 | − |
| | | protein | | | | |
| 12 | FP1.1_CDS_0096 | putative protein | 99 | 7559 | 7657 | + |
| 13 | FP1.1_CDS_0097 | virion structural protein | 1287 | 7723 | 9009 | − |
| 14 | FP1.1_CDS_0098 | terminase large subunit | 1641 | 9042 | 10682 | − |
| 15 | FP1.1_CDS_0099 | putative protein | 684 | 10657 | 11340 | − |
| 16 | FP1.1_CDS_0100 | putative protein | 486 | 11385 | 11870 | − |
| 17 | FP1.1_CDS_0101 | exonuclease | 987 | 11872 | 12858 | − |
| 18 | FP1.1_CDS_0102 | virion structural protein | 600 | 13077 | 13676 | + |
| 19 | FP1.1_CDS_0103 | virion structural protein | 690 | 13691 | 14380 | + |
| 20 | FP1.1_CDS_0104 | virion structural protein | 726 | 14426 | 15151 | − |
| 21 | FP1.1_CDS_0105 | co-chaperonin GroES | 300 | 15144 | 15443 | − |
| 22 | FP1.1_CDS_0106 | putative protein | 207 | 15445 | 15651 | − |
| 23 | FP1.1_CDS_0107 | putative protein | 732 | 15716 | 16447 | − |
| 24 | FP1.1_CDS_0108 | putative protein | 300 | 16550 | 16849 | − |
| 25 | FP1.1_CDS_0109 | putative protein | 150 | 16839 | 16988 | − |
| 26 | FP1.1_CDS_0110 | DNA polymerase | 3006 | 17000 | 20005 | − |
| 27 | FP1.1_CDS_0111 | putative protein | 303 | 20085 | 20387 | − |
| 28 | FP1.1_CDS_0112 | tail fiber assembly protein | 582 | 20411 | 20992 | − |
| 29 | FP1.1_CDS_0113 | tail fiber protein | 1011 | 21003 | 22013 | − |
| 30 | FP1.1_CDS_0114 | tail fiber protein | 663 | 21950 | 22612 | − |
| 31 | FP1.1_CDS_0115 | putative protein | 309 | 22653 | 22961 | − |
| 32 | FP1.1_CDS_0116 | putative protein | 351 | 23001 | 23351 | − |
| 33 | FP1.1_CDS_0117 | endonuclease | 1155 | 23375 | 24529 | − |
| 34 | FP1.1_CDS_0118 | major head protein | 1176 | 24613 | 25788 | − |
| 35 | FP1.1_CDS_0119 | putative protein | 1116 | 25880 | 26995 | − |
| 36 | FP1.1_CDS_0120 | head maturation protease | 624 | 27062 | 27685 | − |
| 37 | FP1.1_CDS_0121 | virion structural protein | 834 | 27706 | 28539 | − |
| 38 | FP1.1_CDS_0122 | putative protein | 228 | 28641 | 28868 | − |
| 39 | FP1.1_CDS_0123 | portal protein | 1740 | 28942 | 30681 | − |
| 40 | FP1.1_CDS_0124 | virion structural protein | 2205 | 30693 | 32897 | − |
| 41 | FP1.1_CDS_0125 | baseplate hub | 831 | 33034 | 33864 | + |
| 42 | FP1.1_CDS_0126 | putative protein | 210 | 33864 | 34073 | + |
| 43 | FP1.1_CDS_0127 | putative protein | 99 | 34143 | 34241 | + |
| 44 | FP1.1_CDS_0128 | putative protein | 138 | 34275 | 34412 | + |
| 45 | FP1.1_CDS_0129 | putative protein | 105 | 34426 | 34530 | + |
| 16 | FP1.1_CDS_0130 | putative protein | 99 | 34702 | 34800 | − |
| 47 | FP1.1_CDS_0131 | homing endonuclease | 948 | 34923 | 35870 | − |
| 48 | FP1.1_CDS_0132 | endonuclease VII | 492 | 35931 | 36422 | + |
| 49 | FP1.1_CDS_0133 | SbcC-like subunit of | 2148 | 36441 | 38588 | − |
| | | palindrome specific | | | | |
| | | endonuclease | | | | |
| 50 | FP1.1_CDS_0134 | SbcD-like subunit of | 1032 | 38597 | 39628 | − |
| | | palindrome specific | | | | |
| | | endonuclease | | | | |
| 51 | FP1.1_CDS_0135 | sigma factor for late | 840 | 39615 | 40454 | − |
| | | transcription | | | | |
| 52 | FP1.1_CDS_0136 | putative protein | 93 | 40461 | 40553 | − |
| 53 | FP1.1_CDS_0137 | virion structural protein | 732 | 40586 | 41317 | + |
| 54 | FP1.1_CDS_0138 | baseplate wedge subunit | 315 | 41317 | 41631 | + |
| 55 | FP1.1_CDS_0139 | virion structural protein | 2250 | 41643 | 43892 | + |
| 56 | FP1.1_CDS_0140 | baseplate hub subunit and tail | 2664 | 43906 | 46569 | + |
| | | lysozyme | | | | |
| 57 | FP1.1_CDS_0141 | baseplate hub subunit and tail | 489 | 46606 | 47094 | + |
| | | lysozymc | | | | |
| 58 | FP1.1_CDS_0142 | baseplate wedge subunit | 396 | 47110 | 47505 | + |
| 59 | FP1.1_CDS_0143 | HNH endonuclcase | 765 | 47575 | 48339 | + |
| 60 | FP1.1_CDS_0144 | baseplate wedge subunit | 3462 | 48344 | 51805 | + |
| 61 | FP1.1_CDS_0145 | virion structural protein | 9972 | 51885 | 61856 | + |
| 62 | FP1.1_CDS_0146 | ATP-dependent protease | 2289 | 61893 | 64181 | − |
| 63 | FP1.1_CDS_0147 | putative protein | 102 | 64194 | 64295 | + |
| 64 | FP1.1_CDS_0148 | virion structural protein | 840 | 64292 | 65131 | + |
| 65 | FP1.1_CDS_0149 | virion structural protein | 564 | 65221 | 65784 | + |
| 66 | FP1.1_CDS_0150 | tail sheath | 1344 | 65781 | 67124 | + |
| 67 | FP1.1_CDS_0151 | MutT/NUDIX hydrolase | 465 | 67138 | 67602 | + |
| 68 | FP1.1_CDS_0152 | virion structural protein | 1101 | 67632 | 68732 | − |
| 69 | FP1.1_CDS_0153 | putative protein | 609 | 68745 | 69353 | − |
| 70 | FP1.1_CDS_0154 | putative protein | 498 | 69363 | 69860 | − |
| 71 | FP1.1_CDS_0155 | tail protein | 1116 | 69971 | 71086 | + |
| 72 | FP1.1_CDS_0156 | colanic acid degradation | 597 | 71101 | 71697 | + |
| 73 | FP1.1_CDS_0157 | virion structural protein | 897 | 71714 | 72610 | + |
| 74 | FP1.1_CDS_0158 | putative protein | 102 | 72591 | 72692 | + |
| 75 | FP1.1_CDS_0159 | tail fiber protein | 5880 | 72748 | 78627 | + |
| 76 | FP1.1_CDS_0160 | virion structural protein | 780 | 78691 | 79470 | + |
| 77 | FP1.1_CDS_0161 | putative protein | 1854 | 79663 | 81516 | + |
| 78 | FP1.1_CDS_0162 | thymidylate synthase | 930 | 81552 | 82481 | − |
| 79 | FP1.1_CDS_0163 | DprA-like DNA recombination- | 594 | 82481 | 83074 | − |
| | | mediator protein | | | | |
| 80 | FP1.1_CDS_0164 | putative protein | 657 | 83078 | 83734 | − |
| 81 | FP1.1_CDS_0165 | putative protein | 246 | 83737 | 83982 | − |
| 82 | FP1.1_CDS_0166 | HNH endonuclease | 1425 | 84092 | 85516 | − |
| 83 | FP1.1_CDS_0167 | ATPase | 1326 | 85591 | 86916 | − |
| 84 | FP1.1_CDS_0168 | putative protein | 195 | 87023 | 87217 | + |
| 85 | FP1.1_CDS_0169 | putative protein | 381 | 87228 | 87608 | − |
| 86 | FP1.1_CDS_0170 | virion structural protein | 396 | 87612 | 88007 | − |
| 87 | FP1.1_CDS_0171 | virion structural protein | 981 | 88009 | 88989 | − |
| 88 | FP1.1_CDS_0172 | virion structural protein | 1518 | 89012 | 90529 | − |
| 89 | FP1.1_CDS_0173 | Hsp70 heat shock protein | 543 | 90543 | 91085 | − |
| 90 | FP1.1_CDS_0174 | virion structural protein | 537 | 91089 | 91625 | − |
| 91 | FP1.1_CDS_0175 | putative protein | 555 | 91653 | 92207 | − |
| 92 | FP1.1_CDS_0176 | head closure | 429 | 92238 | 92666 | − |
| 93 | FP1.1_CDS_0177 | tail tube protein | 1095 | 92666 | 93760 | − |
| 94 | FP1.1_CDS_0178 | tail tube protein | 777 | 93772 | 94548 | − |
| 95 | FP1.1_CDS_0179 | tail tube protein | 687 | 94644 | 95330 | − |
| 96 | FP1.1_CDS_0180 | putative protein | 126 | 95521 | 95646 | − |
| 97 | FP1.1_CDS_0181 | tail sheath | 2664 | 95726 | 98389 | − |
| 98 | FP1.1_CDS_0182 | deoxynucleoside | 627 | 98484 | 99110 | − |
| | | monophosphate kinase | | | | |
| 99 | FP1.1_CDS_0183 | putative protein | 270 | 99129 | 99398 | − |
| 100 | FP1.1_CDS_0184 | head-tail adaptor Ad2 | 792 | 99475 | 100266 | − |
| 101 | FP1.1_CDS_0185 | putative protein | 120 | 100317 | 100436 | − |
| 102 | FP1.1_CDS_0186 | putative protein | 378 | 100528 | 100905 | − |
| 103 | FP1.1_CDS_0187 | putative protein | 309 | 100918 | 101226 | − |
| 104 | FP1.1_CDS_0188 | phosphatase | 477 | 101246 | 101722 | − |
| 105 | FP1.1_CDS_0189 | putative protein | 243 | 101772 | 102014 | − |
| 106 | FP1.1_CDS_0190 | putative protein | 243 | 102061 | 102303 | − |
| 107 | FP1.1_CDS_0191 | thioredoxin domain | 249 | 102303 | 102551 | − |
| 108 | FP1.1_CDS_0192 | nudix hydrolase | 399 | 102553 | 102951 | − |
| 109 | FP1.1_CDS_0193 | tRNA nucleotidyltransferase | 1113 | 103062 | 104174 | − |
| 110 | FP1.1_CDS_0194 | putative protein | 123 | 104176 | 104298 | − |
| 111 | FP1.1_CDS_0195 | putative protein | 375 | 104357 | 104731 | − |
| 112 | FP1.1_CDS_0196 | putative protein | 411 | 104718 | 105128 | − |
| 113 | FP1.1_CDS_0197 | putative protein | 186 | 105125 | 105310 | − |
| 114 | FP1.1_CDS_0198 | putative protein | 396 | 105324 | 105719 | − |
| 115 | FP1.1_CDS_0199 | putative protein | 333 | 105773 | 106105 | − |
| 116 | FP1.1_CDS_0200 | putative protein | 927 | 106122 | 107048 | − |
| 117 | FP1.1_CDS_0201 | putative protein | 138 | 107017 | 107154 | − |
| 118 | FP1.1_CDS_0202 | putative protein | 411 | 107217 | 107627 | − |
| 119 | FP1.1_CDS_0203 | putative protein | 204 | 107635 | 107838 | − |
| 120 | FP1.1_CDS_0204 | putative protein | 132 | 107842 | 107973 | − |
| 121 | FP1.1_CDS_0205 | putative protein | 345 | 107985 | 108329 | − |
| 122 | FP1.1_CDS_0206 | putative protein | 390 | 108359 | 108748 | − |
| 123 | FP1.1_CDS_0207 | putative protein | 453 | 108745 | 109197 | − |
| 124 | FP1.1_CDS_0208 | putative protein | 225 | 109243 | 109467 | − |
| 125 | FP1.1_CDS_0209 | putative protein | 339 | 109460 | 109798 | − |
| 126 | FP1.1_CDS_0210 | putative protein | 480 | 109788 | 110267 | − |
| 127 | FP1.1_CDS_0211 | putative protein | 282 | 110276 | 110557 | − |
| 128 | FP1.1_CDS_0212 | putative protein | 555 | 110575 | 111129 | − |
| 129 | FP1.1_CDS_0213 | putative protein | 393 | 111156 | 111548 | − |
| 130 | FP1.1_CDS_0214 | putative protein | 432 | 111529 | 111960 | − |
| 131 | FP1.1_CDS_0215 | putative protein | 114 | 111961 | 112074 | + |
| 132 | FP1.1_CDS_0216 | starvation-inducible | 441 | 112209 | 112649 | − |
| | | transcriptional regulator | | | | |
| 133 | FP1.1_CDS_0217 | putative protein | 429 | 112682 | 113110 | − |
| 134 | FP1.1_CDS_0218 | putative protein | 216 | 113131 | 113346 | − |
| 135 | FP1.1_CDS_0219 | putative protein | 165 | 113392 | 113556 | − |
| 136 | FP1.1_CDS_0220 | putative protein | 117 | 113580 | 113696 | − |
| 137 | FP1.1_CDS_0221 | putative protein | 477 | 113801 | 114277 | − |
| 138 | FP1.1_CDS_0222 | putative protein | 357 | 114201 | 114557 | − |
| 139 | FP1.1_CDS_0223 | HNH endonuclease | 612 | 114609 | 115220 | − |
| 140 | FP1.1_CDS_0224 | putative protein | 309 | 115222 | 115530 | − |
| 141 | FP1.1_CDS_0225 | Rz-like spanin | 375 | 115527 | 115901 | − |
| 142 | FP1.1_CDS_0226 | putative protein | 420 | 115955 | 116374 | − |
| 143 | FP1.1_CDS_0227 | tail protein | 2790 | 116449 | 119238 | − |
| 144 | FP1.1_CDS_0228 | tail fiber protein | 537 | 119240 | 119776 | − |
| 145 | FP1.1_CDS_0229 | putative protein | 201 | 119739 | 119939 | − |
| 146 | FP1.1_CDS_0230 | nucleotidyltransferase | 2271 | 119945 | 122215 | − |
| 147 | FP1.1_CDS_0231 | putative protein | 402 | 122212 | 122613 | − |
| 148 | FP1.1_CDS_0232 | putative protein | 450 | 122616 | 123065 | − |
| 149 | FP1.1_CDS_0233 | putative protein | 516 | 123121 | 123636 | − |
| 150 | FP1.1_CDS_0234 | putative protein | 567 | 123649 | 124215 | − |
| 151 | FP1.1_CDS_0235 | putative protein | 2730 | 124283 | 127012 | − |
| 152 | FP1.1_CDS_0236 | putative protein | 588 | 127028 | 127615 | − |
| 153 | FP1.1_CDS_0237 | putative protein | 2697 | 127627 | 130323 | − |
| 154 | FP1.1_CDS_0238 | anaerobic ribonucleotide | 471 | 130400 | 130870 | − |
| | | reductase small subunit | | | | |
| 155 | FP1.1_CDS_0239 | unknown function | 333 | 130842 | 131174 | − |
| 156 | FP1.1_CDS_0240 | anaerobic ribonucleoside | 1806 | 131183 | 132988 | − |
| | | reductase large subunit | | | | |
| 157 | FP1.1_CDS_0241 | putative protein | 129 | 133111 | 133239 | − |
| 158 | FP1.1_CDS_0242 | putative protein | 186 | 133252 | 133437 | − |
| 159 | FP1.1_CDS_0243 | putative protein | 411 | 133427 | 133837 | − |
| 160 | FP1.1_CDS_0244 | putative protein | 219 | 133847 | 134065 | − |
| 161 | FP1.1_CDS_0245 | putative protein | 360 | 134066 | 134425 | − |
| 162 | FP1.1_CDS_0246 | putative protein | 225 | 134435 | 134659 | − |
| 163 | FP1.1_CDS_0247 | putative protein | 369 | 134656 | 135024 | − |
| 164 | FP1.1_CDS_0248 | putative protein | 615 | 135021 | 135635 | − |
| 165 | FP1.1_CDS_0249 | putative protein | 360 | 135682 | 136041 | − |
| 166 | FP1.1_CDS_0250 | putative protein | 171 | 136038 | 136208 | − |
| 167 | FP1.1_CDS_0251 | putative protein | 231 | 136205 | 136435 | − |
| 168 | FP1.1_CDS_0252 | putative protein | 180 | 136439 | 136618 | − |
| 169 | FP1.1_CDS_0253 | putative protein | 657 | 136625 | 137281 | − |
| 170 | FP1.1_CDS_0254 | putative protein | 330 | 137281 | 137610 | − |
| 171 | FP1.1_CDS_0255 | putative protein | 558 | 137638 | 138195 | − |
| 172 | FP1.1_CDS_0256 | putative protein | 159 | 138245 | 138403 | − |
| 173 | FP1.1_CDS_0257 | putative protein | 189 | 138422 | 138610 | − |
| 174 | FP1.1_CDS_0258 | PhoH-like phosphate | 1440 | 138679 | 140118 | − |
| | | starvation-inducible | | | | |
| 175 | FP1.1_CDS_0259 | membrane protein | 303 | 140179 | 140481 | − |
| 176 | FP1.1_CDS_0260 | putative protein | 321 | 140478 | 140798 | − |
| 177 | FP1.1_CDS_0261 | nudix hydrolase | 552 | 140808 | 141359 | − |
| 178 | FP1.1_CDS_0262 | Sir2 (NAD-dependent | 753 | 141356 | 142108 | − |
| | | deacetylase) | | | | |
| 179 | FP1.1_CDS_0263 | putative protein | 195 | 142118 | 142312 | − |
| 180 | FP1.1_CDS_0264 | peptidase HslV family | 597 | 142339 | 142935 | − |
| 181 | FP1.1_CDS_0265 | putative protein | 135 | 142932 | 143066 | − |
| 182 | FP1.1_CDS_0266 | putative protein | 147 | 143077 | 143223 | − |
| 183 | FP1.1_CDS_0267 | protease | 609 | 143263 | 143871 | − |
| 184 | FP1.1_CDS_0268 | putative protein | 147 | 143947 | 144093 | − |
| 185 | FP1.1_CDS_0269 | putative protein | 132 | 144053 | 144184 | + |
| 186 | FP1.1_CDS_0270 | virion structural protein | 378 | 144181 | 144558 | + |
| 187 | FP1.1_CDS_0271 | putative protein | 255 | 144587 | 144841 | − |
| 188 | FP1.1_CDS_0272 | dCMP deaminase | 480 | 144838 | 145317 | − |
| 189 | FP1.1_CDS_0273 | virion structural protein | 525 | 145274 | 145798 | − |
| 190 | FP1.1_CDS_0274 | putative protein | 549 | 145770 | 146318 | − |
| 191 | FP1.1_CDS_0275 | putative protein | 99 | 146305 | 146403 | − |
| 192 | FP1.1_CDS_0276 | putative protein | 447 | 146443 | 146889 | − |
| 193 | FP1.1_CDS_0277 | putative protein | 291 | 146891 | 147181 | − |
| 194 | FP1.1_CDS_0278 | head maturation protease | 696 | 147230 | 147925 | − |
| 195 | FP1.1_CDS_0279 | putative protein | 405 | 147990 | 148394 | − |
| 196 | FP1.1_CDS_0280 | putative protein | 384 | 148396 | 148779 | − |
| 197 | FP1.1_CDS_0281 | metallo-phosphoesterase | 792 | 148792 | 149583 | − |
| 198 | FP1.1_CDS_0282 | ATP-dependent DNA ligase | 1419 | 149603 | 151021 | − |
| 199 | FP1.1_CDS_0283 | putative protein | 1200 | 151033 | 152232 | − |
| 200 | FP1.1_CDS_0284 | hydrolase | 414 | 152232 | 152645 | − |
| 201 | FP1.1_CDS_0285 | putative protein | 228 | 152642 | 152869 | − |
| 202 | FP1.1_CDS_0286 | putative protein | 270 | 152880 | 153149 | − |
| 203 | FP1.1_CDS_0287 | NinI-like serine-threonine | 744 | 153155 | 153898 | − |
| | | phosphatase | | | | |
| 204 | FP1.1_CDS_0288 | putative protein | 252 | 153901 | 154152 | − |
| 205 | FP1.1_CDS_0289 | putative protein | 216 | 154152 | 154367 | − |
| 206 | FP1.1_CDS_0290 | putative protein | 330 | 154370 | 154699 | − |
| 207 | FP1.1_CDS_0291 | putative protein | 639 | 154699 | 155337 | − |
| 208 | FP1.1_CDS_0292 | putative protein | 285 | 155315 | 155599 | − |
| 209 | FP1.1_CDS_0293 | putative protein | 306 | 155699 | 156004 | − |
| 210 | FP1.1_CDS_0294 | putative protein | 297 | 156005 | 156301 | − |
| 211 | FP1.1_CDS_0295 | putative protein | 303 | 156298 | 156600 | − |
| 212 | FP1.1_CDS_0296 | putative protein | 252 | 156593 | 156844 | − |
| 213 | FP1.1_CDS_0297 | putative protein | 261 | 156844 | 157104 | − |
| 214 | FP1.1_CDS_0298 | putative protein | 123 | 157104 | 157226 | − |
| 215 | FP1.1_CDS_0299 | putative protein | 99 | 157216 | 157314 | − |
| 216 | FP1.1_CDS_0300 | polynucleotide kinase | 933 | 157330 | 158262 | − |
| 217 | FP1.1_CDS_0301 | putative protein | 315 | 158275 | 158589 | − |
| 218 | FP1.1_CDS_0302 | putative protein | 255 | 158596 | 158850 | − |
| 219 | FP1.1_CDS_0303 | putative protein | 231 | 158834 | 159064 | − |
| 220 | FP1.1_CDS_0304 | putative protein | 255 | 159074 | 159328 | − |
| 221 | FP1.1_CDS_0305 | RNA ligase and tail fiber | 1143 | 159359 | 160501 | − |
| | | protein attachment catalyst | | | | |
| 222 | FP1.1_CDS_0306 | putative protein | 309 | 160498 | 160806 | − |
| 223 | FP1.1_CDS_0307 | putative protein | 690 | 160763 | 161452 | − |
| 224 | FP1.1_CDS_0308 | putative protein | 579 | 161449 | 162027 | − |
| 225 | FP1.1_CDS_0309 | putative protein | 303 | 162035 | 162337 | − |
| 226 | FP1.1_CDS_0310 | putative protein | 282 | 162382 | 162663 | − |
| 227 | FP1.1_CDS_0311 | putative protein | 729 | 162674 | 163402 | − |
| 228 | FP1.1_CDS_0312 | putative protein | 285 | 163461 | 163745 | − |
| 229 | FP1.1_CDS_0313 | putative protein | 183 | 163726 | 163908 | − |
| 230 | FP1.1_CDS_0314 | putative protein | 291 | 163905 | 164195 | − |
| 231 | FP1.1_CDS_0315 | putative protein | 264 | 164192 | 164455 | − |
| 232 | FP1.1_CDS_0316 | putative protein | 201 | 164448 | 164648 | − |
| 233 | FP1.1_CDS_0317 | putative protein | 285 | 164641 | 164925 | − |
| 234 | FP1.1_CDS_0318 | putative protein | 312 | 164888 | 165199 | − |
| 235 | FP1.1_CDS_0319 | putative protein | 312 | 165210 | 165521 | − |
| 236 | FP1.1_CDS_0320 | putative protein | 90 | 165502 | 165591 | − |
| 237 | FP1.1_CDS_0321 | putative protein | 150 | 165627 | 165776 | − |
| 238 | FP1.1_CDS_0322 | putative protein | 276 | 165773 | 166048 | − |
| 239 | FP1.1_CDS_0323 | putative protein | 687 | 166095 | 166781 | − |
| 240 | FP1.1_CDS_0324 | putative protein | 90 | 166830 | 166919 | − |
| 241 | FP1.1_CDS_0325 | putative protein | 294 | 166970 | 167263 | − |
| 242 | FP1.1_CDS_0326 | cytidyltransferase | 534 | 167266 | 167799 | − |
| 243 | FP1.1_CDS_0327 | endonuclease | 786 | 167796 | 168581 | − |
| 244 | FP1.1_CDS_0328 | DNA methyltransferase | 522 | 168578 | 169099 | − |
| 245 | FP1.1_CDS_0329 | putative protein | 543 | 169116 | 169658 | − |
| 246 | FP1.1_CDS_0330 | putative protein | 99 | 169669 | 169767 | + |
| 247 | FP1.1_CDS_0331 | putative protein | 171 | 169788 | 169958 | + |
| 248 | FP1.1_CDS_0332 | putative protein | 90 | 169982 | 170071 | + |
| 249 | FP1.1_CDS_0333 | putative protein | 93 | 170147 | 170239 | + |
| 250 | FP1.1_CDS_0334 | putative protein | 855 | 170340 | 171194 | − |
| 251 | FP1.1_CDS_0335 | putative protein | 306 | 171246 | 171551 | − |
| 252 | FP1.1_CDS_0336 | putative protein | 387 | 171601 | 171987 | − |
| 253 | FP1.1_CDS_0337 | putative protein | 294 | 172059 | 172352 | − |
| 254 | FP1.1_CDS_0338 | putative protein | 849 | 172369 | 173217 | − |
| 255 | FP1.1_CDS_0339 | putative protein | 132 | 173247 | 173378 | − |
| 256 | FP1.1_CDS_0340 | putative protein | 474 | 173381 | 173854 | − |
| 257 | FP1.1_CDS_0341 | putative protein | 498 | 173897 | 174394 | − |
| 258 | FP1.1_CDS_0342 | putative protein | 414 | 174385 | 174798 | − |
| 259 | FP1.1_CDS_0343 | putative protein | 474 | 174746 | 175219 | − |
| 260 | FP1.1_CDS_0344 | putative protein | 537 | 175213 | 175749 | − |
| 261 | FP1.1_CDS_0345 | putative protein | 414 | 175749 | 176162 | − |
| 262 | FP1.1_CDS_0346 | putative protein | 585 | 176172 | 176756 | − |
| 263 | FP1.1_CDS_0347 | putative protein | 603 | 176746 | 177348 | − |
| 264 | FP1.1_CDS_0348 | putative protein | 552 | 177345 | 177896 | − |
| 265 | FP1.1_CDS_0349 | putative protein | 582 | 177958 | 178539 | − |
| 266 | FP1.1_CDS_0350 | putative protein | 510 | 178523 | 179032 | − |
| 267 | FP1.1_CDS_0351 | putative protein | 297 | 179029 | 179325 | − |
| 268 | FP1.1_CDS_0352 | putative protein | 105 | 179352 | 179456 | − |
| 269 | FP1.1_CDS_0353 | putative protein | 582 | 179453 | 180034 | − |
| 270 | FP1.1_CDS_0354 | putative protein | 585 | 180021 | 180605 | − |
| 271 | FP1.1_CDS_0355 | putative protein | 567 | 180602 | 181168 | − |
| 272 | FP1.1_CDS_0356 | putative protein | 90 | 181158 | 181247 | − |
| 273 | FP1.1_CDS_0357 | putative protein | 519 | 181256 | 181774 | − |
| 274 | FP1.1_CDS_0358 | putative protein | 564 | 181767 | 182330 | − |
| 275 | FP1.1_CDS_0359 | putative protein | 543 | 182327 | 182869 | − |
| 276 | FP1.1_CDS_0360 | putative protein | 492 | 182853 | 183344 | − |
| 277 | FP1.1_CDS_0361 | putative protein | 414 | 183344 | 183757 | − |
| 278 | FP1.1_CDS_0362 | putative protein | 363 | 183741 | 184103 | − |
| 279 | FP1.1_CDS_0363 | putative protein | 495 | 184069 | 184563 | − |
| 280 | FP1.1_CDS_0364 | putative protein | 477 | 184560 | 185036 | − |
| 281 | FP1.1_CDS_0365 | putative protein | 537 | 185096 | 185632 | − |
| 282 | FP1.1_CDS_0366 | putative protein | 342 | 185640 | 185981 | − |
| 283 | FP1.1_CDS_0367 | putative protein | 378 | 185960 | 186337 | − |
| 284 | FP1.1_CDS_0368 | putative protein | 282 | 186327 | 186608 | − |
| 285 | FP1.1_CDS_0369 | putative protein | 603 | 186695 | 187297 | − |
| 286 | FP1.1_CDS_0370 | putative protein | 561 | 187288 | 187848 | − |
| 287 | FP1.1_CDS_0371 | putative protein | 135 | 187838 | 187972 | − |
| 288 | FP1.1_CDS_0372 | putative protein | 183 | 188007 | 188189 | − |
| 289 | FP1.1_CDS_0373 | putative protein | 537 | 188203 | 188739 | − |
| 290 | FP1.1_CDS_0374 | putative protein | 447 | 188736 | 189182 | − |
| 291 | FP1.1_CDS_0375 | putative protein | 309 | 189179 | 189487 | − |
| 292 | FP1.1_CDS_0376 | putative protein | 315 | 189487 | 189801 | − |
| 293 | FP1.1_CDS_0377 | putative protein | 453 | 189798 | 190250 | − |
| 294 | FP1.1_CDS_0378 | putative protein | 489 | 190228 | 190716 | − |
| 295 | FP1.1_CDS_0379 | putative protein | 441 | 190719 | 191159 | − |
| 296 | FP1.1_CDS_0380 | putative protein | 471 | 191156 | 191626 | − |
| 297 | FP1.1_CDS_0381 | putative protein | 447 | 191628 | 192074 | − |
| 298 | FP1.1_CDS_0382 | putative protein | 360 | 192132 | 192491 | − |
| 299 | FP1.1_CDS_0383 | putative protein | 357 | 192488 | 192844 | − |
| 300 | FP1.1_CDS_0384 | putative protein | 369 | 192844 | 193212 | − |
| 301 | FP1.1_CDS_0385 | putative protein | 225 | 193209 | 193433 | − |
| 302 | FP1.1_CDS_0386 | putative protein | 339 | 193433 | 193771 | − |
| 303 | FP1.1_CDS_0387 | putative protein | 90 | 193828 | 193917 | − |
| 304 | FP1.1_CDS_0388 | putative protein | 210 | 194007 | 194216 | − |
| 305 | FP1.1_CDS_0389 | putative protein | 210 | 194230 | 194439 | − |
| 306 | FP1.1_CDS_0390 | putative protein | 171 | 194423 | 194593 | − |
| 307 | FP1.1_CDS_0391 | putative protein | 552 | 194615 | 195166 | − |
| 308 | FP1.1_CDS_0392 | putative protein | 345 | 195177 | 195521 | − |
| 309 | FP1.1_CDS_0393 | putative protein | 516 | 195518 | 196033 | − |
| 310 | FP1.1_CDS_0394 | putative protein | 195 | 196042 | 196236 | − |
| 311 | FP1.1_CDS_0395 | putative protein | 615 | 196238 | 196852 | − |
| 312 | FP1.1_CDS_0396 | putative protein | 528 | 196854 | 197381 | − |
| 313 | FP1.1_CDS_0397 | putative protein | 543 | 197378 | 197920 | − |
| 314 | FP1.1_CDS_0398 | putative protein | 507 | 197973 | 198479 | − |
| 315 | FP1.1_CDS_0399 | putative protein | 342 | 198458 | 198799 | − |
| 316 | FP1.1_CDS_0400 | putative protein | 567 | 198786 | 199352 | − |
| 317 | FP1.1_CDS_0401 | putative protein | 312 | 199324 | 199635 | − |
| 318 | FP1.1_CDS_0402 | putative protein | 546 | 199625 | 200170 | − |
| 319 | FP1.1_CDS_0403 | putative protein | 201 | 200179 | 200379 | − |
| 320 | FP1.1_CDS_0404 | putative protein | 162 | 200379 | 200540 | − |
| 321 | FP1.1_CDS_0405 | putative protein | 390 | 200528 | 200917 | − |
| 322 | FP1.1_CDS_0406 | putative protein | 267 | 200889 | 201155 | − |
| 323 | FP1.1_CDS_0407 | putative protein | 216 | 201125 | 201340 | − |
| 324 | FP1.1_CDS_0408 | putative protein | 321 | 201318 | 201638 | − |
| 325 | FP1.1_CDS_0409 | putative protein | 246 | 201635 | 201880 | − |
| 326 | FP1.1_CDS_0410 | putative protein | 345 | 201971 | 202315 | − |
| 327 | FP1.1_CDS_0411 | putative protein | 261 | 202305 | 202565 | − |
| 328 | FP1.1_CDS_0412 | putative protein | 222 | 202549 | 202770 | − |
| 329 | FP1.1_CDS_0413 | putative protein | 216 | 202767 | 202982 | − |
| 330 | FP1.1_CDS_0414 | putative protein | 216 | 202979 | 203194 | − |
| 331 | FP1.1_CDS_0415 | putative protein | 216 | 203179 | 203394 | − |
| 332 | FP1.1_CDS_0416 | putative protein | 222 | 203391 | 203612 | − |
| 333 | FP1.1_CDS_0417 | putative protein | 231 | 203590 | 203820 | − |
| 334 | FP1.1_CDS_0418 | putative protein | 246 | 203801 | 204046 | − |
| 335 | FP1.1_CDS_0419 | putative protein | 243 | 204036 | 204278 | − |
| 336 | FP1.1_CDS_0420 | putative protein | 237 | 204262 | 204498 | − |
| 337 | FP1.1_CDS_0421 | putative protein | 216 | 204499 | 204714 | − |
| 338 | FP1.1_CDS_0422 | putative protein | 267 | 204707 | 204973 | − |
| 339 | FP1.1_CDS_0423 | putative protein | 231 | 204975 | 205205 | − |
| 340 | FP1.1_CDS_0424 | putative protein | 219 | 205202 | 205420 | − |
| 341 | FP1.1_CDS_0425 | putative protein | 219 | 205411 | 205629 | − |
| 342 | FP1.1_CDS_0426 | putative protein | 216 | 205626 | 205841 | − |
| 343 | FP1.1_CDS_0427 | putative protein | 252 | 205825 | 206076 | − |
| 344 | FP1.1_CDS_0428 | putative protein | 252 | 206061 | 206312 | − |
| 345 | FP1.1_CDS_0429 | putative protein | 258 | 206290 | 206547 | − |
| 346 | FP1.1_CDS_0430 | putative protein | 159 | 206544 | 206702 | − |
| 347 | FP1.1_CDS_0431 | putative protein | 147 | 206702 | 206848 | − |
| 348 | FP1.1_CDS_0432 | putative protein | 228 | 206912 | 207139 | − |
| 349 | FP1.1_CDS_0433 | putative protein | 216 | 207136 | 207351 | − |
| 350 | FP1.1_CDS_0434 | putative protein | 213 | 207348 | 207560 | − |
| 351 | FP1.1_CDS_0435 | putative protein | 210 | 207557 | 207766 | − |
| 352 | FP1.1_CDS_0436 | putative protein | 201 | 207763 | 207963 | − |
| 353 | FP1.1_CDS_0437 | putative protein | 228 | 207960 | 208187 | − |
| 354 | FP1.1_CDS_0438 | putative protein | 210 | 208407 | 208616 | − |
| 355 | FP1.1_CDS_0439 | putative protein | 402 | 208685 | 209086 | − |
| 356 | FP1.1_CDS_0440 | putative protein | 327 | 209176 | 209502 | − |
| 357 | FP1.1_CDS_0441 | putative protein | 132 | 209504 | 209635 | + |
| 358 | FP1.1_CDS_0442 | putative protein | 201 | 209757 | 209957 | − |
| 359 | FP1.1_CDS_0443 | putative protein | 258 | 210046 | 210303 | − |
| 360 | FP1.1_CDS_0444 | putative protein | 225 | 210378 | 210602 | − |
| 361 | FP1.1_CDS_0445 | putative protein | 192 | 210686 | 210877 | − |
| 362 | FP1.1_CDS_0446 | putative protein | 273 | 210874 | 211146 | − |
| 363 | FP1.1_CDS_0447 | putative protein | 255 | 211231 | 211485 | − |
| 364 | FP1.1_CDS_0448 | putative protein | 102 | 211556 | 211657 | − |
| 365 | FP1.1_CDS_0449 | putative protein | 585 | 211676 | 212260 | − |
| 366 | FP1.1_CDS_0450 | putative protein | 243 | 212292 | 212534 | − |
| 367 | FP1.1_CDS_0451 | putative protein | 255 | 212588 | 212842 | − |
| 368 | FP1.1_CDS_0452 | putative protein | 396 | 212905 | 213300 | − |
| 369 | FP1.1_CDS_0453 | putative protein | 324 | 213400 | 213723 | − |
| 370 | FP1.1_CDS_0454 | putative protein | 135 | 213810 | 213944 | − |
| 371 | FP1.1_CDS_0455 | putative protein | 96 | 213999 | 214094 | − |
| 372 | FP1.1_CDS_0456 | putative protein | 159 | 214141 | 214299 | − |
| 373 | FP1.1_CDS_0457 | putative protein | 102 | 214333 | 214434 | − |
| 374 | FP1.1_CDS_0458 | putative protein | 150 | 214484 | 214633 | − |
| 375 | FP1.1_CDS_0459 | putative protein | 171 | 214630 | 214800 | − |
| 376 | FP1.1_CDS_0460 | putative protein | 93 | 214797 | 214889 | − |
| 377 | FP1.1_CDS_0461 | putative protein | 213 | 214907 | 215119 | − |
| 378 | FP1.1_CDS_0462 | putative protein | 324 | 215203 | 215526 | − |
| 379 | FP1.1_CDS_0463 | putative protein | 189 | 215554 | 215742 | − |
| 380 | FP1.1_CDS_0464 | acetyltransferase | 357 | 215822 | 216178 | − |
| 381 | FP1.1_CDS_0465 | DksA-like zinc-finger protein | 258 | 216242 | 216499 | − |
| 382 | FP1.1_CDS_0466 | putative protein | 117 | 216499 | 216615 | − |
| 383 | FP1.1_CDS_0467 | putative protein | 171 | 216603 | 216773 | + |
| 384 | FP1.1_CDS_0468 | putative protein | 198 | 216831 | 217028 | − |
| 385 | FP1.1_CDS_0469 | putative protein | 144 | 217134 | 217277 | − |
| 386 | FP1.1_CDS_0470 | putative protein | 180 | 217377 | 217556 | − |
| 387 | FP1.1_CDS_0471 | putative protein | 213 | 217648 | 217860 | − |
| 388 | FP1.1_CDS_0472 | putative protein | 342 | 217953 | 218294 | − |
| 389 | FP1.1_CDS_0473 | putative protein | 102 | 218404 | 218505 | − |
| 390 | FP1.1_CDS_0474 | putative protein | 138 | 218515 | 218652 | − |
| 391 | FP1.1_CDS_0475 | putative protein | 90 | 218653 | 218742 | − |
| 392 | FP1.1_CDS_0476 | putative protein | 153 | 218739 | 218891 | − |
| 393 | FP1.1_CDS_0477 | putative protein | 207 | 218957 | 219163 | − |
| 394 | FP1.1_CDS_0478 | putative protein | 171 | 219165 | 219335 | − |
| 395 | FP1.1_CDS_0479 | putative protein | 270 | 219387 | 219656 | − |
| 396 | FP1.1_CDS_0480 | putative protein | 324 | 219737 | 220060 | − |
| 397 | FP1.1_CDS_0481 | putative protein | 144 | 220150 | 220293 | − |
| 398 | FP1.1_CDS_0482 | putative protein | 228 | 220379 | 220606 | − |
| 399 | FP1.1_CDS_0483 | putative protein | 153 | 220677 | 220829 | − |
| 400 | FP1.1_CDS_0484 | putative protein | 93 | 220920 | 221012 | + |
| 401 | FP1.1_CDS_0485 | putative protein | 483 | 221105 | 221587 | − |
| 402 | FP1.1_CDS_0486 | putative protein | 123 | 221671 | 221793 | − |
| 403 | FP1.1_CDS_0487 | putative protein | 300 | 221826 | 222125 | − |
| 404 | FP1.1_CDS_0488 | putative protein | 114 | 222177 | 222290 | − |
| 405 | FP1.1_CDS_0489 | putative protein | 222 | 222372 | 222593 | − |
| 406 | FP1.1_CDS_0490 | putative protein | 189 | 222677 | 222865 | − |
| 407 | FP1.1_CDS_0491 | putative protein | 213 | 222915 | 223127 | − |
| 408 | FP1.1_CDS_0492 | putative protein | 285 | 223145 | 223429 | − |
| 409 | FP1.1_CDS_0493 | putative protein | 240 | 223488 | 223727 | − |
| 410 | FP1.1_CDS_0494 | putative protein | 237 | 223819 | 224055 | − |
| 411 | FP1.1_CDS_0495 | putative protein | 612 | 224114 | 224725 | − |
| 412 | FP1.1_CDS_0496 | putative protein | 96 | 224782 | 224877 | − |
| 413 | FP1.1_CDS_0497 | putative protein | 171 | 224924 | 225094 | − |
| 414 | FP1.1_CDS_0498 | putative protein | 231 | 225152 | 225382 | − |
| 415 | FP1.1_CDS_0499 | putative protein | 267 | 225470 | 225736 | − |
| 416 | FP1.1_CDS_0500 | putative protein | 240 | 225820 | 226059 | − |
| 417 | FP1.1_CDS_0501 | putative protein | 363 | 226154 | 226516 | − |
| 418 | FP1.1_CDS_0502 | putative protein | 297 | 226534 | 226830 | + |
| 419 | FP1.1_CDS_0503 | putative protein | 147 | 226898 | 227044 | + |
| 420 | FP1.1_CDS_0504 | putative protein | 114 | 227114 | 227227 | + |
| 421 | FP1.1_CDS_0505 | putative protein | 93 | 227224 | 227316 | − |
| 422 | FP1.1_CDS_0506 | putative protein | 168 | 227660 | 227827 | − |
| 423 | FP1.1_CDS_0507 | putative protein | 126 | 227859 | 227984 | − |
| 424 | FP1.1_CDS_0508 | putative protein | 129 | 228176 | 228304 | + |
| 425 | FP1.1_CDS_0509 | putative protein | 201 | 228510 | 228710 | − |
| 426 | FP1.1_CDS_0510 | putative protein | 186 | 228716 | 228901 | − |
| 427 | FP1.1_CDS_0511 | putative protein | 186 | 228903 | 229088 | − |
| 428 | FP1.1_CDS_0512 | putative protein | 189 | 229098 | 229286 | − |
| 429 | FP1.1_CDS_0513 | putative protein | 183 | 229288 | 229470 | − |
| 430 | FP1.1_CDS_0514 | putative protein | 192 | 229472 | 229663 | − |
| 431 | FP1.1_CDS_0515 | putative protein | 180 | 229672 | 229851 | − |
| 432 | FP1.1_CDS_0516 | putative protein | 183 | 229853 | 230035 | − |
| 433 | FP1.1_CDS_0517 | putative protein | 183 | 230046 | 230228 | − |
| 434 | FP1.1_CDS_0518 | putative protein | 660 | 230221 | 230880 | − |
| 435 | FP1.1_CDS_0519 | putative protein | 180 | 230891 | 231070 | − |
| 436 | FP1.1_CDS_0520 | putative protein | 189 | 231079 | 231267 | − |
| 437 | FP1.1_CDS_0521 | putative protein | 183 | 231275 | 231457 | − |
| 438 | FP1.1_CDS_0522 | putative protein | 186 | 231466 | 231651 | − |
| 439 | FP1.1_CDS_0523 | putative protein | 195 | 231699 | 231893 | − |
| 440 | FP1.1_CDS_0524 | putative protein | 210 | 231886 | 232095 | − |
| 441 | FP1.1_CDS_0525 | putative protein | 204 | 232097 | 232300 | − |
| 442 | FP1.1_CDS_0526 | putative protein | 228 | 232297 | 232524 | − |
| 443 | FP1.1_CDS_0527 | regulator of chromosome | 1149 | 232627 | 233775 | + |
| | | condensation | | | | |
| 444 | FP1.1_CDS_0528 | regulator of chromosome | 1182 | 233848 | 235029 | + |
| | | condensation | | | | |
| 445 | FP1.1_CDS_0529 | regulator of chromosome | 1131 | 235105 | 236235 | + |
| | | condensation | | | | |
| 446 | FP1.1_CDS_0530 | regulator of chromosome | 1203 | 236246 | 237448 | + |
| | | condensation | | | | |
| 447 | FP1.1_CDS_0531 | regulator of chromosome | 1110 | 237457 | 238566 | + |
| | | condensation | | | | |
| 448 | FP1.1_CDS_0532 | regulator of chromosome | 1188 | 238638 | 239825 | + |
| | | condensation | | | | |
| 449 | FP1.1_CDS_0533 | putative protein | 90 | 239897 | 239986 | + |
| 450 | FP1.1_CDS_0534 | regulator of chromosome | 1074 | 240002 | 241075 | + |
| | | condensation | | | | |
| 451 | FP1.1_CDS_0535 | regulator of chromosome | 1152 | 241147 | 242298 | + |
| | | condensation | | | | |
| 452 | FP1.1_CDS_0536 | regulator of chromosome | 1176 | 242370 | 243545 | + |
| | | condensation | | | | |
| 453 | FP1.1_CDS_0537 | regulator of chromosome | 1206 | 243621 | 244826 | + |
| | | condensation | | | | |
| 454 | FP1.1_CDS_0538 | regulator of chromosome | 225 | 244899 | 245123 | + |
| | | condensation | | | | |
| 455 | FP1.1_CDS_0539 | regulator of chromosome | 897 | 245155 | 246051 | + |
| | | condensation | | | | |
| 456 | FP1.1_CDS_0540 | putative protein | 660 | 246032 | 246691 | − |
| 457 | FP1.1_CDS_0541 | putative protein | 372 | 246736 | 247107 | − |
| 458 | FP1.1_CDS_0542 | putative protein | 363 | 247109 | 247471 | − |
| 459 | FP1.1_CDS_0543 | putative protein | 369 | 247473 | 247841 | − |
| 460 | FP1.1_CDS_0544 | putative protein | 315 | 247841 | 248155 | − |
| 461 | FP1.1_CDS_0545 | putative protein | 336 | 248155 | 248490 | − |
| 462 | FP1.1_CDS_0546 | putative protein | 348 | 248490 | 248837 | − |
| 463 | FP1.1_CDS_0547 | putative protein | 339 | 248837 | 249175 | − |
| 464 | FP1.1_CDS_0548 | putative protein | 342 | 249175 | 249516 | − |
| 465 | FP1.1_CDS_0549 | unknown function | 771 | 249584 | 250354 | − |
| 466 | FP1.1_CDS_0550 | putative protein | 114 | 250332 | 250445 | + |
| 467 | FP1.1_CDS_0551 | endonuclease | 915 | 250438 | 251352 | + |
| 468 | FP1.1_CDS_0552 | endonuclease | 990 | 251373 | 252362 | − |
| 469 | FP1.1_CDS_0553 | endonuclease | 636 | 252308 | 252943 | − |
| 470 | FP1.1_CDS_0554 | putative protein | 240 | 252933 | 253172 | − |
| 471 | FP1.1_CDS_0555 | putative protein | 360 | 253238 | 253597 | − |
| 472 | FP1.1_CDS_0556 | putative protein | 195 | 253704 | 253898 | − |
| 473 | FP1.1_CDS_0557 | endonuclease V N-glycosylase | 516 | 253937 | 254452 | − |
| | | UV repair enzyme | | | | |
| 474 | FP1.1_CDS_0558 | putative protein | 372 | 254541 | 254912 | − |
| 475 | FP1.1_CDS_0559 | putative protein | 252 | 254923 | 255174 | − |
| 476 | FP1.1_CDS_0560 | putative protein | 177 | 255203 | 255379 | − |
| 477 | FP1.1_CDS_0561 | putative protein | 555 | 255391 | 255945 | − |
| 478 | FP1.1_CDS_0562 | putative protein | 396 | 256022 | 256417 | − |
| 479 | FP1.1_CDS_0563 | putative protein | 255 | 256402 | 256656 | − |
| 480 | FP1.1_CDS_0564 | putative protein | 345 | 256666 | 257010 | − |
| 481 | FP1.1_CDS_0565 | putative protein | 342 | 257012 | 257353 | − |
| 482 | FP1.1_CDS_0566 | putative protein | 339 | 257363 | 257701 | − |
| 483 | FP1.1_CDS_0567 | putative protein | 282 | 257704 | 257985 | − |
| 484 | FP1.1_CDS_0568 | protein kinase | 642 | 258047 | 258688 | − |
| 485 | FP1.1_CDS_0569 | virion structural protein | 1539 | 258759 | 260297 | − |
| 486 | FP1.1_CDS_0570 | HNH endonuclease | 555 | 260302 | 260856 | − |
| 487 | FP1.1_CDS_0571 | putative protein | 297 | 260907 | 261203 | − |
| 488 | FP1.1_CDS_0572 | putative protein | 264 | 261200 | 261463 | − |
| 489 | FP1.1_CDS_0573 | putative protein | 114 | 261472 | 261585 | − |
| 490 | FP1.1_CDS_0574 | virion structural protein | 207 | 261673 | 261879 | − |
| 491 | FP1.1_CDS_0575 | putative protein | 312 | 261995 | 262306 | − |
| 492 | FP1.1_CDS_0576 | putative protein | 156 | 262281 | 262436 | − |
| 493 | FP1.1_CDS_0577 | putative protein | 573 | 262450 | 263022 | − |
| 494 | FP1.1_CDS_0578 | putative protein | 222 | 263030 | 263251 | − |
| 495 | FP1.1_CDS_0579 | putative protein | 294 | 263304 | 263597 | − |
| 496 | FP1.1_CDS_0580 | putative protein | 267 | 263609 | 263875 | − |
| 497 | FP1.1_CDS_0581 | putative protein | 345 | 263890 | 264234 | − |
| 498 | FP1.1_CDS_0582 | unknown function | 441 | 264227 | 264667 | − |
| 499 | FP1.1_CDS_0583 | putative protein | 192 | 264722 | 264913 | − |
| 500 | FP1.1_CDS_0584 | putative protein | 351 | 264918 | 265268 | − |
| 501 | FP1.1_CDS_0585 | putative protein | 378 | 265271 | 265648 | − |
| 502 | FP1.1_CDS_0586 | putative protein | 360 | 265659 | 266018 | − |
| 503 | FP1.1_CDS_0587 | putative protein | 327 | 266097 | 266423 | − |
| 504 | FP1.1_CDS_0588 | putative protein | 240 | 266484 | 266723 | − |
| 505 | FP1.1_CDS_0589 | putative protein | 663 | 266714 | 267376 | − |
| 506 | FP1.1_CDS_0590 | putative protein | 207 | 267408 | 267614 | − |
| 507 | FP1.1_CDS_0591 | putative protein | 219 | 267616 | 267834 | − |
| 508 | FP1.1_CDS_0592 | putative protein | 222 | 267838 | 268059 | − |
| 509 | FP1.1_CDS_0593 | putative protein | 519 | 268056 | 268574 | − |
| 510 | FP1.1_CDS_0594 | putative protein | 201 | 268574 | 268774 | − |
| 511 | FP1.1_CDS_0595 | putative protein | 147 | 268783 | 268929 | − |
| 512 | FP1.1_CDS_0596 | putative protein | 216 | 268926 | 269141 | − |
| 513 | FP1.1_CDS_0597 | putative protein | 276 | 269141 | 269416 | − |
| 514 | FP1.1_CDS_0598 | putative protein | 369 | 269413 | 269781 | − |
| 515 | FP1.1_CDS_0599 | putative protein | 252 | 269778 | 270029 | − |
| 516 | FP1.1_CDS_0600 | putative protein | 309 | 270029 | 270337 | − |
| 517 | FP1.1_CDS_0601 | putative protein | 279 | 270456 | 270734 | − |
| 518 | FP1.1_CDS_0602 | putative protein | 201 | 270904 | 271104 | − |
| 519 | FP1.1_CDS_0603 | putative protein | 183 | 271115 | 271297 | − |
| 520 | FP1.1_CDS_0604 | putative protein | 252 | 271315 | 271566 | + |
| 521 | FP1.1_CDS_0605 | putative protein | 117 | 271547 | 271663 | + |
| 522 | FP1.1_CDS_0606 | putative protein | 240 | 271660 | 271899 | + |
| 523 | FP1.1_CDS_0607 | putative protein | 162 | 271980 | 272141 | + |
| 524 | FP1.1_CDS_0608 | putative protein | 111 | 272113 | 272223 | − |
| 525 | FP1.1_CDS_0609 | RNA polymerase sigma factor | 966 | 272234 | 273199 | − |
| 526 | FP1.1_CDS_0610 | putative protein | 498 | 273221 | 273718 | − |
| 527 | FP1.1_CDS_0611 | putative protein | 420 | 273720 | 274139 | − |
| 528 | FP1.1_CDS_0612 | putative protein | 261 | 274145 | 274405 | − |
| 529 | FP1.1_CDS_0613 | putative protein | 252 | 274480 | 274731 | − |
| 530 | FP1.1_CDS_0614 | putative protein | 426 | 274791 | 275216 | − |
| 531 | FP1.1_CDS_0615 | membrane protein | 423 | 275213 | 275635 | − |
| 532 | FP1.1_CDS_0616 | putative protein | 459 | 275637 | 276095 | − |
| 533 | FP1.1_CDS_0617 | putative protein | 477 | 276137 | 276613 | − |
| 534 | FP1.1_CDS_0618 | putative protein | 150 | 276613 | 276762 | − |
| 535 | FP1.1_CDS_0619 | putative protein | 270 | 276798 | 277067 | − |
| 536 | FP1.1_CDS_0620 | putative protein | 228 | 277070 | 277297 | − |
| 537 | FP1.1_CDS_0621 | putative protein | 585 | 277294 | 277878 | − |
| 538 | FP1.1_CDS_0622 | virion structural protein | 1155 | 277995 | 279149 | + |
| 539 | FP1.1_CDS_0623 | virion structural protein | 789 | 279192 | 279980 | − |
| 540 | FP1.1_CDS_0624 | putative protein | 342 | 279994 | 280335 | − |
| 541 | FP1.1_CDS_0625 | putative protein | 111 | 280336 | 280446 | − |
| 542 | FP1.1_CDS_0626 | putative protein | 372 | 280481 | 280852 | − |
| 543 | FP1.1_CDS_0627 | putative protein | 420 | 280852 | 281271 | − |
| 544 | FP1.1_CDS_0628 | putative protein | 447 | 281283 | 281729 | − |
| 545 | FP1.1_CDS_0629 | putative protein | 453 | 281719 | 282171 | − |
| 546 | FP1.1_CDS_0630 | putative protein | 429 | 282119 | 282547 | − |
| 547 | FP1.1_CDS_0631 | putative protein | 420 | 282537 | 282956 | − |
| 548 | FP1.1_CDS_0632 | putative protein | 108 | 283038 | 283145 | − |
| 549 | FP1.1_CDS_0633 | putative protein | 285 | 283190 | 283474 | − |
| 550 | FP1.1_CDS_0634 | putative protein | 192 | 283474 | 283665 | − |
| 551 | FP1.1_CDS_0635 | putative protein | 366 | 283675 | 284040 | − |
| 552 | FP1.1_CDS_0636 | putative protein | 426 | 284033 | 284458 | − |
| 553 | FP1.1_CDS_0637 | putative protein | 114 | 284626 | 284739 | − |
| 554 | FP1.1_CDS_0638 | putative protein | 216 | 284726 | 284941 | − |
| 555 | FP1.1_CDS_0639 | putative protein | 90 | 285005 | 285094 | − |
| 556 | FP1.1_CDS_0640 | putative protein | 216 | 285141 | 285356 | − |
| 557 | FP1.1_CDS_0641 | putative protein | 402 | 285369 | 285770 | − |
| 558 | FP1.1_CDS_0642 | putative protein | 258 | 285779 | 286036 | − |
| 559 | FP1.1_CDS_0643 | putative protein | 186 | 286046 | 286231 | − |
| 560 | FP1.1_CDS_0644 | HNH endonuclease | 564 | 286341 | 286904 | − |
| 561 | FP1.1_CDS_0645 | HNH endonuclease | 114 | 286930 | 287043 | − |
| 562 | FP1.1_CDS_0646 | putative protein | 435 | 287262 | 287696 | − |
| 563 | FP1.1_CDS_0647 | putative protein | 354 | 287786 | 288139 | − |
| 564 | FP1.1_CDS_0648 | putative protein | 165 | 288210 | 288374 | − |
| 565 | FP1.1_CDS_0649 | putative protein | 195 | 288371 | 288565 | − |
| 566 | FP1.1_CDS_0650 | putative protein | 168 | 288532 | 288699 | − |
| 567 | FP1.1_CDS_0651 | HNH endonuclease | 537 | 288702 | 289238 | − |
| 568 | FP1.1_CDS_0652 | putative protein | 105 | 289485 | 289589 | − |
| 569 | FP1.1_CDS_0653 | putative protein | 177 | 289606 | 289782 | − |
| 570 | FP1.1_CDS_0654 | putative protein | 504 | 289779 | 290282 | − |
| 571 | FP1.1_CDS_0655 | putative protein | 183 | 290328 | 290510 | − |
| 572 | FP1.1_CDS_0656 | putative protein | 162 | 290537 | 290698 | − |
| 573 | FP1.1_CDS_0657 | putative protein | 99 | 290702 | 290800 | − |
| 574 | FP1.1_CDS_0658 | putative protein | 153 | 290872 | 291024 | − |
| 575 | FP1.1_CDS_0659 | putative protein | 405 | 291047 | 291451 | − |
| 576 | FP1.1_CDS_0660 | putative protein | 489 | 291448 | 291936 | − |
| 577 | FP1.1_CDS_0661 | putative protein | 219 | 291938 | 292156 | − |
| 578 | FP1.1_CDS_0662 | putative protein | 393 | 292205 | 292597 | − |
| 579 | FP1.1_CDS_0663 | putative protein | 150 | 292652 | 292801 | − |
| 580 | FP1.1_CDS_0664 | putative protein | 291 | 292824 | 293114 | − |
| 581 | FP1.1_CDS_0665 | membrane protein | 507 | 293114 | 293620 | − |
| 582 | FP1.1_CDS_0666 | putative protein | 210 | 293686 | 293895 | − |
| 583 | FP1.1_CDS_0667 | putative protein | 297 | 294011 | 294307 | − |
| 584 | FP1.1_CDS_0668 | putative protein | 270 | 294300 | 294569 | − |
| 585 | FP1.1_CDS_0669 | putative protein | 291 | 294569 | 294859 | − |
| 586 | FP1.1_CDS_0670 | putative protein | 831 | 294856 | 295686 | − |
| 587 | FP1.1_CDS_0671 | putative protein | 198 | 295689 | 295886 | − |
| 588 | FP1.1_CDS_0672 | putative protein | 573 | 295873 | 296445 | − |
| 589 | FP1.1_CDS_0673 | putative protein | 144 | 296503 | 296646 | − |
| 590 | FP1.1_CDS_0674 | putative protein | 171 | 296646 | 296816 | − |
| 591 | FP1.1_CDS_0675 | putative protein | 144 | 296833 | 296976 | − |
| 592 | FP1.1_CDS_0676 | putative protein | 444 | 296979 | 297422 | − |
| 593 | FP1.1_CDS_0677 | putative protein | 384 | 297419 | 297802 | − |
| 594 | FP1.1_CDS_0678 | putative protein | 456 | 297802 | 298257 | − |
| 595 | FP1.1_CDS_0679 | putative protein | 516 | 298254 | 298769 | − |
| 596 | FP1.1_CDS_0680 | putative protein | 531 | 298812 | 299342 | − |
| 597 | FP1.1_CDS_0681 | putative protein | 216 | 299294 | 299509 | − |
| 598 | FP1.1_CDS_0682 | anaerobic ribonucleoside | 1611 | 299572 | 301182 | − |
| | | reductase large subunit | | | | |
| 599 | FP1.1_CDS_0683 | putative protein | 252 | 301235 | 301486 | − |
| 600 | FP1.1_CDS_0684 | putative protein | 90 | 301479 | 301568 | − |
| 601 | FP1.1_CDS_0685 | putative protein | 261 | 301565 | 301825 | − |
| 602 | FP1.1_CDS_0686 | putative protein | 441 | 301840 | 302280 | − |
| 603 | FP1.1_CDS_0687 | putative protein | 456 | 302274 | 302729 | − |
| 604 | FP1.1_CDS_0688 | putative protein | 378 | 302726 | 303103 | − |
| 605 | FP1.1_CDS_0689 | putative protein | 405 | 303105 | 303509 | − |
| 606 | FP1.1_CDS_0690 | putative protein | 411 | 303511 | 303921 | − |
| 607 | FP1.1_CDS_0691 | putative protein | 546 | 303977 | 304522 | − |
| 608 | FP1.1_CDS_0692 | putative protein | 552 | 304534 | 305085 | − |
| 609 | FP1.1_CDS_0693 | putative protein | 429 | 305139 | 305567 | − |
| 610 | FP1.1_CDS_0694 | putative protein | 129 | 305571 | 305699 | − |
| 611 | FP1.1_CDS_0695 | virion structural protein | 216 | 305761 | 305976 | − |
| 612 | FP1.1_CDS_0696 | virion structural protein | 153 | 305943 | 306095 | − |
| 613 | FP1.1_CDS_0697 | putative protein | 423 | 306092 | 306514 | − |
| 614 | FP1.1_CDS_0698 | putative protein | 162 | 306511 | 306672 | − |
| 615 | FP1.1_CDS_0699 | putative protein | 231 | 306674 | 306904 | − |
| 616 | FP1.1_CDS_0700 | putative protein | 279 | 306885 | 307163 | − |
| 617 | FP1.1_CDS_0701 | putative protein | 492 | 307156 | 307647 | − |
| 618 | FP1.1_CDS_0702 | putative protein | 354 | 307650 | 308003 | − |
| 619 | FP1.1_CDS_0703 | putative protein | 573 | 308007 | 308579 | − |
| 620 | FP1.1_CDS_0704 | putative protein | 597 | 308576 | 309172 | − |
| 621 | FP1.1_CDS_0705 | virion structural protein | 555 | 309207 | 309761 | − |
| 622 | FP1.1_CDS_0706 | putative protein | 1095 | 309758 | 310852 | − |
| 623 | FP1.1_CDS_0707 | exonuclease | 690 | 310856 | 311545 | − |
| 624 | FP1.1_CDS_0708 | tellurite resistance | 1143 | 311618 | 312760 | − |
| 625 | FP1.1_CDS_0709 | putative protein | 102 | 312761 | 312862 | − |
| 626 | FP1.1_CDS_0710 | putative protein | 1128 | 312846 | 313973 | − |
| 627 | FP1.1_CDS_0711 | putative protein | 819 | 314070 | 314888 | − |
| 628 | FP1.1_CDS_0712 | virion structural protein | 681 | 314885 | 315565 | − |
| 629 | FP1.1_CDS_0713 | virion structural protein | 564 | 315565 | 316128 | − |
| 630 | FP1.1_CDS_0714 | unknown function | 627 | 316225 | 316851 | − |
| 631 | FP1.1_CDS_0715 | clamp loader of DNA | 726 | 316851 | 317576 | − |
| | | polymerase | | | | |
| 632 | FP1.1_CDS_0716 | clamp loader of DNA | 216 | 317588 | 317803 | − |
| | | polymerase | | | | |
| 633 | FP1.1_CDS_0717 | putative protein | 270 | 317827 | 318096 | − |
| 634 | FP1.1_CDS_0718 | head morphogenesis | 327 | 318153 | 318479 | − |
| 635 | FP1.1_CDS_0719 | putative protein | 345 | 318488 | 318832 | − |
| 636 | FP1.1_CDS_0720 | DNA polymerase | 543 | 318896 | 319438 | − |
| 637 | FP1.1_CDS_0721 | putative protein | 312 | 319374 | 319685 | − |
| 638 | FP1.1_CDS_0722 | virion structural protein | 684 | 319687 | 320370 | − |
| 639 | FP1.1_CDS_0723 | virion structural protein | 1719 | 320361 | 322079 | − |
| 640 | FP1.1_CDS_0724 | DNA topoisomerase II | 1413 | 322168 | 323580 | − |
| 641 | FP1.1_CDS_0725 | DNA topoisomerase II large | 930 | 323631 | 324560 | − |
| | | subunit | | | | |
| 642 | FP1.1_CDS_0726 | DNA topoisomerase II large | 1038 | 324557 | 325594 | − |
| | | subunit | | | | |
| 643 | FP1.1_CDS_0727 | virion structural protein | 792 | 325688 | 326479 | − |
| 644 | FP1.1_CDS_0728 | virion structural protein | 1572 | 326479 | 328050 | − |
| 645 | FP1.1_CDS_0729 | putative protein | 252 | 328040 | 328291 | − |
| 646 | FP1.1_CDS_0730 | putative protein | 309 | 328276 | 328584 | − |
| 647 | FP1.1_CDS_0731 | ATP-dependent protease | 546 | 328656 | 329201 | − |
| 648 | FP1.1_CDS_0732 | translation initiation factor | 504 | 329215 | 329718 | − |
| 649 | FP1.1_CDS_0733 | putative protein | 195 | 329788 | 329982 | − |
| 650 | FP1.1_CDS_0734 | putative protein | 567 | 330006 | 330572 | − |
| 651 | FP1.1_CDS_0735 | Dda-like helicase | 1341 | 330656 | 331996 | − |
| 652 | FP1.1_CDS_0736 | Rnase H | 429 | 332027 | 332455 | − |
| 653 | FP1.1_CDS_0737 | dihydrofolate reductase | 522 | 332452 | 332973 | − |
| 654 | FP1.1_CDS_0738 | putative protein | 369 | 332961 | 333329 | − |
| 655 | FP1.1_CDS_0739 | putative protein | 132 | 333323 | 333454 | − |
| 656 | FP1.1_CDS_0740 | membrane protein | 663 | 333478 | 334140 | + |
| 657 | FP1.1_CDS_0741 | virion structural protein | 318 | 334157 | 334474 | + |
| 658 | FP1.1_CDS_0742 | putative protein | 339 | 334479 | 334817 | + |
| 659 | FP1.1_CDS_0743 | nucleotide | 732 | 334853 | 335584 | − |
| | | pyrophosphohydrolase | | | | |
| 660 | FP1.1_CDS_0744 | virion structural protein | 396 | 335584 | 335979 | − |
| 661 | FP1.1_CDS_0745 | putative protein | 234 | 335990 | 336223 | − |
| 662 | FP1.1_CDS_0746 | membrane protein | 168 | 336286 | 336453 | − |
| 663 | FP1.1_CDS_0747 | aerobic NDP reductase small | 351 | 336504 | 336854 | − |
| | | subunit | | | | |
| 664 | FP1.1_CDS_0748 | ribonucleoside diphosphate | 780 | 336848 | 337627 | − |
| | | reductase small subunit | | | | |
| 665 | FP1.1_CDS_0749 | ribonucleotide reductase large | 2319 | 337682 | 340000 | − |
| | | subunit | | | | |
| 666 | FP1.1_CDS_0750 | virion structural protein | 582 | 340064 | 340645 | − |
| 667 | FP1.1_CDS_0751 | putative protein | 555 | 340674 | 341228 | − |
| 668 | FP1.1_CDS_0752 | exonuclease | 720 | 341215 | 341934 | − |
| 669 | FP1.1_CDS_0753 | virion structural protein | 336 | 341974 | 342309 | − |
| 670 | FP1.1_CDS_0754 | virion structural protein | 633 | 342326 | 342958 | − |
| 671 | FP1.1_CDS_0755 | putative protein | 672 | 342992 | 343663 | − |
| 672 | FP1.1_CDS_0756 | virion structural protein | 219 | 343660 | 343878 | − |
| 673 | FP1.1_CDS_0757 | virion structural protein | 945 | 343878 | 344822 | − |
| 674 | FP1.1_CDS_0758 | virion structural protein | 393 | 344879 | 345271 | − |
| 675 | FP1.1_CDS_0759 | putative protein | 165 | 345268 | 345432 | − |
| 676 | FP1.1_CDS_0760 | virion structural protein | 273 | 345434 | 345706 | − |
| 677 | FP1.1_CDS_0761 | DnaB-like replicative helicase | 1521 | 345710 | 347230 | − |
| 678 | FP1.1_CDS_0762 | DNA primase | 1056 | 347253 | 348308 | − |
| 679 | FP1.1_CDS_0763 | virion structural protein | 978 | 348364 | 349341 | − |
| 680 | FP1.1_CDS_0764 | putative protein | 354 | 349399 | 349752 | + |
| 681 | FP1.1_CDS_0765 | DNA polymerase | 639 | 349739 | 350377 | − |
| 682 | FP1.1_CDS_0766 | virion structural protein | 330 | 350355 | 350684 | − |

TABLE 3
Protein-encoding genes from the genome of the bacteriophage FP1.2.

| SEQ ID | | | Gene | | | |
|---|---|---|---|---|---|---|
| NO | Gene_id | Annotation | length | Start | End | Strand |
| 683 | FP1.2_CDS_0001 | NAD-dependent DNA ligase | 2049 | 3 | 2051 | + |
| 684 | FP1.2_CDS_0002 | putative protein | 321 | 2044 | 2364 | + |
| 685 | FP1.2_CDS_0003 | putative protein | 480 | 2377 | 2856 | + |
| 686 | FP1.2_CDS_0004 | putative protein | 504 | 2859 | 3362 | + |
| 687 | FP1.2_CDS_0005 | putative protein | 504 | 3337 | 3840 | + |
| 688 | FP1.2_CDS_0006 | putative protein | 531 | 3812 | 4342 | + |
| 689 | FP1.2_CDS_0007 | putative protein | 507 | 4276 | 4782 | + |
| 690 | FP1.2_CDS_0008 | acetyltransferase | 534 | 4779 | 5312 | + |
| 691 | FP1.2_CDS_0009 | putative protein | 429 | 5314 | 5742 | + |
| 692 | FP1.2_CDS_0010 | putative protein | 651 | 5793 | 6443 | + |
| 693 | FP1.2_CDS_0011 | putative protein | 726 | 6415 | 7140 | + |
| 694 | FP1.2_CDS_0012 | putative protein | 600 | 7185 | 7784 | + |
| 695 | FP1.2_CDS_0013 | putative protein | 414 | 7784 | 8197 | + |
| 696 | FP1.2_CDS_0014 | putative protein | 447 | 8203 | 8649 | + |
| 697 | FP1.2_CDS_0015 | putative protein | 1026 | 8588 | 9613 | + |
| 698 | FP1.2_CDS_0016 | putative protein | 318 | 9722 | 10039 | + |
| 699 | FP1.2_CDS_0017 | putative protein | 423 | 10098 | 10520 | + |
| 700 | FP1.2_CDS_0018 | putative protein | 351 | 10559 | 10909 | + |
| 701 | FP1.2_CDS_0019 | putative protein | 210 | 10928 | 11137 | + |
| 702 | FP1.2_CDS_0020 | putative protein | 171 | 11130 | 11300 | + |
| 703 | FP1.2_CDS_0021 | putative protein | 192 | 11314 | 11505 | + |
| 704 | FP1.2_CDS_0022 | putative protein | 372 | 11522 | 11893 | + |
| 705 | FP1.2_CDS_0023 | putative protein | 321 | 11862 | 12182 | + |
| 706 | FP1.2_CDS_0024 | putative protein | 168 | 12200 | 12367 | + |
| 707 | FP1.2_CDS_0025 | endolysin | 498 | 12436 | 12933 | + |
| 708 | FP1.2_CDS_0026 | putative protein | 105 | 12945 | 13049 | + |
| 709 | FP1.2_CDS_0027 | putative protein | 249 | 13027 | 13275 | + |
| 710 | FP1.2_CDS_0028 | putative protein | 750 | 13451 | 14200 | + |
| 711 | FP1.2_CDS_0029 | putative protein | 576 | 14197 | 14772 | + |
| 712 | FP1.2_CDS_0030 | putative protein | 831 | 14909 | 15739 | + |
| 713 | FP1.2_CDS_0031 | tail protein | 3597 | 15753 | 19349 | + |
| 714 | FP1.2_CDS_0032 | putative protein | 1770 | 19373 | 21142 | + |
| 715 | FP1.2_CDS_0033 | putative protein | 96 | 21118 | 21213 | + |
| 716 | FP1.2_CDS_0034 | putative protein | 528 | 21296 | 21823 | + |
| 717 | FP1.2_CDS_0035 | tail fiber protein | 3525 | 21830 | 25354 | + |
| 718 | FP1.2_CDS_0036 | putative protein | 507 | 25403 | 25909 | + |
| 719 | FP1.2_CDS_0037 | putative protein | 333 | 25972 | 26304 | + |
| 720 | FP1.2_CDS_0038 | putative protein | 642 | 26408 | 27049 | + |
| 721 | FP1.2_CDS_0039 | putative protein | 2613 | 27072 | 29684 | + |
| 722 | FP1.2_CDS_0040 | putative protein | 570 | 29706 | 30275 | + |
| 723 | FP1.2_CDS_0041 | putative protein | 375 | 30285 | 30659 | + |
| 724 | FP1.2_CDS_0042 | phosphatase | 561 | 30725 | 31285 | − |
| 725 | FP1.2_CDS_0043 | putative protein | 2085 | 31236 | 33320 | − |
| 726 | FP1.2_CDS_0044 | putative protein | 366 | 33317 | 33682 | − |
| 727 | FP1.2_CDS_0045 | putative protein | 96 | 33839 | 33934 | − |
| 728 | FP1.2_CDS_0046 | putative protein | 873 | 33886 | 34758 | + |
| 729 | FP1.2_CDS_0047 | RNA polymerase beta subunit | 1302 | 34772 | 36073 | + |
| 730 | FP1.2_CDS_0048 | putative protein | 627 | 36099 | 36725 | + |
| 731 | FP1.2_CDS_0049 | putative protein | 117 | 36827 | 36943 | + |
| 732 | FP1.2_CDS_0050 | putative protein | 402 | 36940 | 37341 | + |
| 733 | FP1.2_CDS_0051 | putative protein | 129 | 37375 | 37503 | + |
| 734 | FP1.2_CDS_0052 | putative protein | 426 | 37516 | 37941 | + |
| 735 | FP1.2_CDS_0053 | putative protein | 603 | 38040 | 38642 | + |
| 736 | FP1.2_CDS_0054 | putative protein | 495 | 38687 | 39181 | + |
| 737 | FP1.2_CDS_0055 | putative protein | 459 | 39253 | 39711 | + |
| 738 | FP1.2_CDS_0056 | putative protein | 465 | 39708 | 40172 | + |
| 739 | FP1.2_CDS_0057 | putative protein | 768 | 40175 | 40942 | + |
| 740 | FP1.2_CDS_0058 | putative protein | 396 | 40975 | 41370 | + |
| 741 | FP1.2_CDS_0059 | putative protein | 399 | 41408 | 41806 | + |
| 742 | FP1.2_CDS_0060 | putative protein | 465 | 41855 | 42319 | + |
| 743 | FP1.2_CDS_0061 | putative protein | 486 | 42390 | 42875 | + |
| 744 | FP1.2_CDS_0062 | putative protein | 105 | 42856 | 42960 | + |
| 745 | FP1.2_CDS_0063 | DNA polymerase | 1752 | 42962 | 44713 | − |
| 746 | FP1.2_CDS_0064 | putative protein | 1311 | 44935 | 46245 | + |
| 747 | FP1.2_CDS_0065 | putative protein | 441 | 46278 | 46718 | + |
| 748 | FP1.2_CDS_0066 | putative protein | 2796 | 46715 | 49510 | + |
| 749 | FP1.2_CDS_0067 | putative protein | 2793 | 49570 | 52362 | + |
| 750 | FP1.2_CDS_0068 | putative protein | 2994 | 52425 | 55418 | − |
| 751 | FP1.2_CDS_0069 | putative protein | 1206 | 55422 | 56627 | − |
| 752 | FP1.2_CDS_0070 | putative protein | 1107 | 56662 | 57768 | + |
| 753 | FP1.2_CDS_0071 | putative protein | 903 | 57808 | 58710 | + |
| 754 | FP1.2_CDS_0072 | virion structural protein | 531 | 58707 | 59237 | + |
| 755 | FP1.2_CDS_0073 | putative protein | 1323 | 59302 | 60624 | + |
| 756 | FP1.2_CDS_0074 | putative protein | 1317 | 60727 | 62043 | + |
| 757 | FP1.2_CDS_0075 | putative protein | 1290 | 62053 | 63342 | + |
| 758 | FP1.2_CDS_0076 | putative protein | 1302 | 63491 | 64792 | + |
| 759 | FP1.2_CDS_0077 | putative protein | 1149 | 64734 | 65882 | + |
| 760 | FP1.2_CDS_0078 | putative protein | 3378 | 65898 | 69275 | + |
| 761 | FP1.2_CDS_0079 | putative protein | 111 | 69257 | 69367 | + |
| 762 | FP1.2_CDS_0080 | putative protein | 384 | 69364 | 69747 | + |
| 763 | FP1.2_CDS_0081 | putative protein | 1566 | 69740 | 71305 | + |
| 764 | FP1.2_CDS_0082 | virion structural protein | 1317 | 71292 | 72608 | + |
| 765 | FP1.2_CDS_0083 | putative protein | 606 | 72605 | 73210 | + |
| 766 | FP1.2_CDS_0084 | putative protein | 1368 | 73213 | 74580 | + |
| 767 | FP1.2_CDS_0085 | putative protein | 981 | 74514 | 75494 | + |
| 768 | FP1.2_CDS_0086 | putative protein | 903 | 75580 | 76482 | + |
| 769 | FP1.2_CDS_0087 | putative protein | 570 | 76558 | 77127 | + |
| 770 | FP1.2_CDS_0088 | putative protein | 402 | 77198 | 77599 | + |
| 771 | FP1.2_CDS_0089 | putative protein | 327 | 77606 | 77932 | + |
| 772 | FP1.2_CDS_0090 | DnaB-like replicative helicase | 1611 | 78005 | 79615 | − |
| 773 | FP1.2_CDS_0091 | putative protein | 621 | 79655 | 80275 | + |
| 774 | FP1.2_CDS_0092 | putative protein | 114 | 80312 | 80425 | − |
| 775 | FP1.2_CDS_0093 | putative protein | 2301 | 80422 | 82722 | − |
| 776 | FP1.2_CDS_0094 | putative protein | 105 | 82828 | 82932 | − |
| 777 | FP1.2_CDS_0095 | putative protein | 1437 | 82985 | 84421 | + |
| 778 | FP1.2_CDS_0096 | polymerase | 1797 | 84461 | 86257 | + |
| 779 | FP1.2_CDS_0097 | putative protein | 2559 | 86292 | 88850 | + |
| 780 | FP1.2_CDS_0098 | putative protein | 273 | 88912 | 89184 | + |
| 781 | FP1.2_CDS_0099 | virion structural protein | 2910 | 89269 | 92178 | − |
| 782 | FP1.2_CDS_0100 | putative protein | 2412 | 92206 | 94617 | + |
| 783 | FP1.2_CDS_0101 | virion structural protein | 936 | 94677 | 95612 | + |
| 784 | FP1.2_CDS_0102 | virion structural protein | 852 | 95623 | 96474 | + |
| 785 | FP1.2_CDS_0103 | putative protein | 885 | 96491 | 97375 | + |
| 786 | FP1.2_CDS_0104 | virion structural protein | 906 | 97391 | 98296 | + |
| 787 | FP1.2_CDS_0105 | putative protein | 1281 | 98348 | 99628 | − |
| 788 | FP1.2_CDS_0106 | virion structural protein | 2154 | 99733 | 101886 | + |
| 789 | FP1.2_CDS_0107 | putative protein | 510 | 101944 | 102453 | + |
| 790 | FP1.2_CDS_0108 | putative protein | 486 | 102458 | 102943 | + |
| 791 | FP1.2_CDS_0109 | putative protein | 2598 | 102956 | 105553 | + |
| 792 | FP1.2_CDS_0110 | virion structural protein | 2049 | 105567 | 107615 | + |
| 793 | FP1.2_CDS_0111 | endolysin | 636 | 107742 | 108377 | + |
| 794 | FP1.2_CDS_0112 | putative protein | 111 | 108448 | 108558 | − |
| 795 | FP1.2_CDS_0113 | putative protein | 90 | 108555 | 108644 | − |
| 796 | FP1.2_CDS_0114 | virion structural protein | 600 | 108672 | 109271 | − |
| 797 | FP1.2_CDS_0115 | putative protein | 93 | 109282 | 109374 | − |
| 798 | FP1.2_CDS_0116 | virion structural protein | 894 | 109356 | 110249 | + |
| 799 | FP1.2_CDS_0117 | putative protein | 111 | 110376 | 110486 | + |
| 800 | FP1.2_CDS_0118 | putative protein | 156 | 110558 | 110713 | − |
| 801 | FP1.2_CDS_0119 | putative protein | 783 | 111040 | 111822 | + |
| 802 | FP1.2_CDS_0120 | putative protein | 300 | 111819 | 112118 | + |
| 803 | FP1.2_CDS_0121 | putative protein | 276 | 112159 | 112434 | + |
| 804 | FP1.2_CDS_0122 | putative protein | 408 | 112512 | 112919 | + |
| 805 | FP1.2_CDS_0123 | putative protein | 444 | 113032 | 113475 | + |
| 806 | FP1.2_CDS_0124 | putative protein | 426 | 113491 | 113916 | + |
| 807 | FP1.2_CDS_0125 | putative protein | 363 | 113918 | 114280 | + |
| 808 | FP1.2_CDS_0126 | putative protein | 390 | 114386 | 114775 | + |
| 809 | FP1.2_CDS_0127 | baseplate assembly protein | 1089 | 114854 | 115942 | − |
| 810 | FP1.2_CDS_0128 | SbcC-like subunit of | 2520 | 115994 | 118513 | + |
| | | palindrome specific | | | | |
| | | endonuclease | | | | |
| 811 | FP1.2_CDS_0129 | putative protein | 813 | 118535 | 119347 | + |
| 812 | FP1.2_CDS_0130 | putative protein | 642 | 119317 | 119958 | + |
| 813 | FP1.2_CDS_0131 | putative protein | 1227 | 119992 | 121218 | + |
| 814 | FP1.2_CDS_0132 | thymidylate kinase | 630 | 121235 | 121864 | + |
| 815 | FP1.2_CDS_0133 | putative protein | 351 | 121918 | 122268 | − |
| 816 | FP1.2_CDS_0134 | putative protein | 123 | 122337 | 122459 | − |
| 817 | FP1.2_CDS_0135 | putative protein | 822 | 122563 | 123384 | − |
| 818 | FP1.2_CDS_0136 | putative protein | 123 | 123350 | 123472 | + |
| 819 | FP1.2_CDS_0137 | putative protein | 660 | 123535 | 124194 | + |
| 820 | FP1.2_CDS_0138 | putative protein | 528 | 124292 | 124819 | + |
| 821 | FP1.2_CDS_0139 | putative protein | 414 | 124831 | 125244 | + |
| 822 | FP1.2_CDS_0140 | putative protein | 702 | 125259 | 125960 | + |
| 823 | FP1.2_CDS_0141 | putative protein | 627 | 126068 | 126694 | − |
| 824 | FP1.2_CDS_0142 | virion structural protein | 1377 | 126697 | 128073 | − |
| 825 | FP1.2_CDS_0143 | putative protein | 552 | 128092 | 128643 | + |
| 826 | FP1.2_CDS_0144 | ribonuclease | 1527 | 128694 | 130220 | + |
| 827 | FP1.2_CDS_0145 | putative protein | 432 | 130224 | 130655 | + |
| 828 | FP1.2_CDS_0146 | putative protein | 843 | 130707 | 131549 | − |
| 829 | FP1.2_CDS_0147 | UvsX-like recombinase | 1491 | 131648 | 133138 | + |
| 830 | FP1.2_CDS_0148 | putative protein | 321 | 133151 | 133471 | + |
| 831 | FP1.2_CDS_0149 | GTP-binding domain | 327 | 133540 | 133866 | + |
| 832 | FP1.2_CDS_0150 | RNA polymerase beta subunit | 675 | 133876 | 134550 | + |
| 833 | FP1.2_CDS_0151 | putative protein | 708 | 134553 | 135260 | + |
| 834 | FP1.2_CDS_0152 | putative protein | 789 | 135257 | 136045 | + |
| 835 | FP1.2_CDS_0153 | putative protein | 630 | 135948 | 136577 | − |
| 836 | FP1.2_CDS_0154 | putative protein | 99 | 136579 | 136677 | − |
| 837 | FP1.2_CDS_0155 | putative protein | 516 | 136711 | 137226 | + |
| 838 | FP1.2_CDS_0156 | putative protein | 513 | 137223 | 137735 | + |
| 839 | FP1.2_CDS_0157 | putative protein | 1068 | 137818 | 138885 | + |
| 840 | FP1.2_CDS_0158 | putative protein | 2142 | 138941 | 141082 | − |
| 841 | FP1.2_CDS_0159 | tail fiber protein | 6666 | 141132 | 147797 | − |
| 842 | FP1.2_CDS_0160 | putative protein | 219 | 147832 | 148050 | + |
| 843 | FP1.2_CDS_0161 | RNA polymerase beta subunit | 1647 | 148043 | 149689 | + |
| 844 | FP1.2_CDS_0162 | RNA polymerase beta subunit | 4347 | 149693 | 154039 | + |
| 845 | FP1.2_CDS_0163 | putative protein | 234 | 154141 | 154374 | + |
| 846 | FP1.2_CDS_0164 | putative protein | 1524 | 154374 | 155897 | + |
| 847 | FP1.2_CDS_0165 | virion structural protein | 711 | 155898 | 156608 | + |
| 848 | FP1.2_CDS_0166 | putative protein | 732 | 156610 | 157341 | + |
| 849 | FP1.2_CDS_0167 | head maturation protease | 810 | 157420 | 158229 | + |
| 850 | FP1.2_CDS_0168 | putative protein | 111 | 158248 | 158358 | + |
| 851 | FP1.2_CDS_0169 | putative protein | 801 | 158512 | 159312 | + |
| 852 | FP1.2_CDS_0170 | putative protein | 480 | 159448 | 159927 | + |
| 853 | FP1.2_CDS_0171 | putative protein | 453 | 159993 | 160445 | + |
| 854 | FP1.2_CDS_0172 | putative protein | 540 | 160445 | 160984 | + |
| 855 | FP1.2_CDS_0173 | virion structural protein | 891 | 161057 | 161947 | − |
| 856 | FP1.2_CDS_0174 | tail sheath | 2073 | 161963 | 164035 | − |
| 857 | FP1.2_CDS_0175 | putative protein | 948 | 164179 | 165126 | + |
| 858 | FP1.2_CDS_0176 | putative protein | 2517 | 165185 | 167701 | + |
| 859 | FP1.2_CDS_0177 | virion structural protein | 1734 | 167704 | 169437 | + |
| 860 | FP1.2_CDS_0178 | terminase large subunit | 2232 | 169488 | 171719 | + |
| 861 | FP1.2_CDS_0179 | putative protein | 309 | 171805 | 172113 | + |
| 862 | FP1.2_CDS_0180 | putative protein | 132 | 172157 | 172288 | + |
| 863 | FP1.2_CDS_0181 | putative protein | 1239 | 172307 | 173545 | − |
| 864 | FP1.2_CDS_0182 | putative protein | 552 | 173657 | 174208 | + |
| 865 | FP1.2_CDS_0183 | putative protein | 600 | 174205 | 174804 | + |
| 866 | FP1.2_CDS_0184 | putative protein | 354 | 174789 | 175142 | + |
| 867 | FP1.2_CDS_0185 | putative protein | 135 | 175211 | 175345 | + |
| 868 | FP1.2_CDS_0186 | putative protein | 1038 | 175451 | 176488 | + |
| 869 | FP1.2_CDS_0187 | putative protein | 825 | 176466 | 177290 | + |
| 870 | FP1.2_CDS_0188 | putative protein | 375 | 177304 | 177678 | + |
| 871 | FP1.2_CDS_0189 | putative protein | 300 | 177691 | 177990 | + |
| 872 | FP1.2_CDS_0190 | putative protein | 537 | 177987 | 178523 | + |
| 873 | FP1.2_CDS_0191 | putative protein | 843 | 178514 | 179356 | − |
| 874 | FP1.2_CDS_0192 | tubulin PhuZ | 981 | 179421 | 180401 | + |
| 875 | FP1.2_CDS_0193 | putative protein | 222 | 180401 | 180622 | + |
| 876 | FP1.2_CDS_0194 | putative protein | 321 | 180693 | 181013 | + |
| 877 | FP1.2_CDS_0195 | putative protein | 99 | 181010 | 181108 | + |
| 878 | FP1.2_CDS_0196 | putative protein | 978 | 181105 | 182082 | + |
| 879 | FP1.2_CDS_0197 | putative protein | 651 | 182112 | 182762 | + |
| 880 | FP1.2_CDS_0198 | putative protein | 513 | 182744 | 183256 | + |
| 881 | FP1.2_CDS_0199 | putative protein | 483 | 183225 | 183707 | + |
| 882 | FP1.2_CDS_0200 | putative protein | 423 | 183668 | 184090 | + |
| 883 | FP1.2_CDS_0201 | putative protein | 414 | 184077 | 184490 | + |
| 884 | FP1.2_CDS_0202 | putative protein | 153 | 184589 | 184741 | + |
| 885 | FP1.2_CDS_0203 | DNA polymerase | 2154 | 184790 | 186943 | + |
| 886 | FP1.2_CDS_0204 | putative protein | 1095 | 187010 | 188104 | − |
| 887 | FP1.2_CDS_0205 | putative protein | 126 | 188068 | 188193 | + |
| 888 | FP1.2_CDS_0206 | putative protein | 96 | 188199 | 188294 | + |
| 889 | FP1.2_CDS_0207 | nuclear shell protein | 1896 | 188359 | 190254 | + |
| 890 | FP1.2_CDS_0208 | RNA polymerase beta subunit | 1305 | 190347 | 191651 | + |
| 891 | FP1.2_CDS_0209 | putative protein | 186 | 191677 | 191862 | + |
| 892 | FP1.2_CDS_0210 | putative protein | 225 | 191966 | 192190 | + |
| 893 | FP1.2_CDS_0211 | putative protein | 639 | 192206 | 192844 | + |
| 894 | FP1.2_CDS_0212 | putative protein | 837 | 192888 | 193724 | + |
| 895 | FP1.2_CDS_0213 | putative protein | 465 | 193844 | 194308 | + |
| 896 | FP1.2_CDS_0214 | putative protein | 126 | 194277 | 194402 | + |
| 897 | FP1.2_CDS_0215 | putative protein | 450 | 194428 | 194877 | + |
| 898 | FP1.2_CDS_0216 | putative protein | 408 | 194891 | 195298 | + |
| 899 | FP1.2_CDS_0217 | putative protein | 783 | 195279 | 196061 | + |
| 900 | FP1.2_CDS_0218 | SbcD-like subunit of | 1143 | 196061 | 197203 | + |
| | | palindrome specific | | | | |
| | | endonuclease | | | | |
| 901 | FP1.2_CDS_0219 | putative protein | 798 | 197223 | 198020 | + |
| 902 | FP1.2_CDS_0220 | putative protein | 792 | 198094 | 198885 | + |
| 903 | FP1.2_CDS_0221 | putative protein | 3282 | 198981 | 202262 | + |
| 904 | FP1.2_CDS_0222 | putative protein | 1281 | 202318 | 203598 | + |
| 905 | FP1.2_CDS_0223 | putative protein | 270 | 203664 | 203933 | − |
| 906 | FP1.2_CDS_0224 | RNA polymerase beta subunit | 2334 | 203884 | 206217 | + |
| 907 | FP1.2_CDS_0225 | putative protein | 411 | 206229 | 206639 | + |
| 908 | FP1.2_CDS_0226 | putative protein | 423 | 206686 | 207108 | + |
| 909 | FP1.2_CDS_0227 | putative protein | 363 | 207132 | 207494 | + |
| 910 | FP1.2_CDS_0228 | putative protein | 96 | 207542 | 207637 | + |
| 911 | FP1.2_CDS_0229 | putative protein | 486 | 207652 | 208137 | + |
| 912 | FP1.2_CDS_0230 | RNA polymerase beta subunit | 2007 | 208283 | 210289 | + |
| 913 | FP1.2_CDS_0231 | DNA helicase | 2262 | 210286 | 212547 | + |
| 914 | FP1.2_CDS_0232 | putative protein | 894 | 212616 | 213509 | + |
| 915 | FP1.2_CDS_0233 | putative protein | 339 | 213506 | 213844 | + |
| 916 | FP1.2_CDS_0234 | putative protein | 1455 | 213914 | 215368 | − |
| 917 | FP1.2_CDS_0235 | DNA helicase | 2082 | 215469 | 217550 | − |
| 918 | FP1.2_CDS_0236 | putative protein | 99 | 217534 | 217632 | + |
| 919 | FP1.2_CDS_0237 | putative protein | 99 | 217664 | 217762 | + |
| 920 | FP1.2_CDS_0238 | putative protein | 687 | 217759 | 218445 | + |
| 921 | FP1.2_CDS_0239 | putative protein | 483 | 218468 | 218950 | + |
| 922 | FP1.2_CDS_0240 | phosphoesterase | 537 | 218968 | 219504 | + |
| 923 | FP1.2_CDS_0241 | putative protein | 276 | 219571 | 219846 | + |
| 924 | FP1.2_CDS_0242 | putative protein | 462 | 219896 | 220357 | + |
| 925 | FP1.2_CDS_0243 | putative protein | 522 | 220412 | 220933 | + |
| 926 | FP1.2_CDS_0244 | putative protein | 264 | 220949 | 221212 | + |
| 927 | FP1.2_CDS_0245 | putative protein | 957 | 221212 | 222168 | + |
| 928 | FP1.2_CDS_0246 | putative protein | 777 | 222250 | 223026 | + |
| 929 | FP1.2_CDS_0247 | putative protein | 303 | 223023 | 223325 | + |
| 930 | FP1.2_CDS_0248 | putative protein | 597 | 223309 | 223905 | + |
| 931 | FP1.2_CDS_0249 | putative protein | 342 | 223902 | 224243 | + |
| 932 | FP1.2_CDS_0250 | exonuclease | 1245 | 224254 | 225498 | + |
| 933 | FP1.2_CDS_0251 | putative protein | 315 | 225550 | 225864 | + |
| 934 | FP1.2_CDS_0252 | putative protein | 300 | 225928 | 226227 | + |
| 935 | FP1.2_CDS_0253 | putative protein | 534 | 226305 | 226838 | + |
| 936 | FP1.2_CDS_0254 | putative protein | 549 | 226925 | 227473 | + |
| 937 | FP1.2_CDS_0255 | putative protein | 969 | 227497 | 228465 | + |
| 938 | FP1.2_CDS_0256 | putative protein | 204 | 228539 | 228742 | + |
| 939 | FP1.2_CDS_0257 | thymidylate synthase | 1020 | 228739 | 229758 | + |
| 940 | FP1.2_CDS_0258 | putative protein | 261 | 229839 | 230099 | + |
| 941 | FP1.2_CDS_0259 | putative protein | 147 | 230130 | 230276 | + |
| 942 | FP1.2_CDS_0260 | putative protein | 204 | 230269 | 230472 | + |
| 943 | FP1.2_CDS_0261 | putative protein | 198 | 230499 | 230696 | + |
| 944 | FP1.2_CDS_0262 | putative protein | 318 | 230717 | 231034 | + |
| 945 | FP1.2_CDS_0263 | putative protein | 399 | 231031 | 231429 | + |
| 946 | FP1.2_CDS_0264 | putative protein | 642 | 231420 | 232061 | + |
| 947 | FP1.2_CDS_0265 | putative protein | 369 | 232048 | 232416 | + |
| 948 | FP1.2_CDS_0266 | putative protein | 327 | 232428 | 232754 | + |
| 949 | FP1.2_CDS_0267 | putative protein | 309 | 232760 | 233068 | + |
| 950 | FP1.2_CDS_0268 | putative protein | 294 | 233087 | 233380 | + |
| 951 | FP1.2_CDS_0269 | putative protein | 486 | 233373 | 233858 | + |
| 952 | FP1.2_CDS_0270 | putative protein | 255 | 233831 | 234085 | + |
| 953 | FP1.2_CDS_0271 | putative protein | 375 | 234092 | 234466 | + |
| 954 | FP1.2_CDS_0272 | putative protein | 117 | 234544 | 234660 | + |
| 955 | FP1.2_CDS_0273 | putative protein | 267 | 234657 | 234923 | + |
| 956 | FP1.2_CDS_0274 | putative protein | 978 | 234938 | 235915 | + |
| 957 | FP1.2_CDS_0275 | putative protein | 138 | 235895 | 236032 | − |
| 958 | FP1.2_CDS_0276 | putative protein | 231 | 236119 | 236349 | + |
| 959 | FP1.2_CDS_0277 | DNA methyltransferase | 486 | 236330 | 236815 | + |
| 960 | FP1.2_CDS_0278 | DNA methyltransferase | 300 | 236854 | 237153 | + |
| 961 | FP1.2_CDS_0279 | putative protein | 294 | 237161 | 237454 | + |
| 962 | FP1.2_CDS_0280 | putative protein | 621 | 237417 | 238037 | + |
| 963 | FP1.2_CDS_0281 | putative protein | 96 | 238053 | 238148 | + |
| 964 | FP1.2_CDS_0282 | putative protein | 285 | 238145 | 238429 | + |

Interestingly, most of the genes in the bacteriophage genomes are unrelated to known proteins and cannot be assigned a function based on homology searches. Given their large genome size, the phage genomes of the invention may harbor novel genes useful to counteract host protection systems such as anti-restriction, RNA repair proteins or CRISPR (anti-CRISPR Acr), or genes that are involved in mechanisms responsible for phage genomic variations such as novel polymerases or DNA modification-enzymes.

In some embodiments, a recombinant bacteriophage according to the invention comprises (i). a genome having an overall sequence identity with at least 70% of the genome of the wild-type bacteriophage deposited under Accession No. NCIMB44022; or (ii). a genome including the nucleotide sequence of SEQ ID NO: 1, or a genome including at least 70% overall sequence identity with the nucleotide sequence of SEQ ID NO:1; or (iii). a genome including an overall sequence identity with at least 70% of the genome of the wild-type bacteriophage deposited under Accession No. NCIMB44023; or (iv). a genome including the nucleotide sequence of SEQ ID NO: 2, or a genome including at least 70% overall sequence identity with the nucleotide sequence of SEQ ID NO:2, wherein said genome further comprises at least one heterologous nucleic acid encoding for a protein/polypeptide which enhances the substantial lytic activity of said recombinant bacteriophage compared to that of the wild-type bacteriophage. In one aspect of the embodiment, the at least one heterologous nucleic acid incorporated to the genome of the recombinant bacteriophage above encodes for a protein/polypeptide which inactivates the immune system of the bacterial host. In another aspect of the embodiment, the at least one heterologous nucleic acid incorporated to the genome of the recombinant bacteriophage above encodes for a protein/polypeptide which induces the degradation of the bacterial host genome.

The nucleic acid of the invention may be isolated from the phage genome or bacteria genome using standard methods for nucleic acid purification (such as affinity purification with commercially available silica columns) and/or produced using molecular biology tools (such as cloning), and/or enzymatic or chemical synthesis approaches known in the art.

### Biocontrol Compositions

The invention refers to a biocontrol composition containing at least one of the bacteriophages, and a carrier. In particular, the invention relates to a biocontrol composition including the bacteriophage FP1.1 or the bacteriophage FP1.2; or a combination of both FP1.1 and FP1.2, referred to as FP1. The biocontrol compositions of the invention comprise a bacteriophage used alone or in combination with others, parts of others, and/or parts of their nucleic acids or gene products.

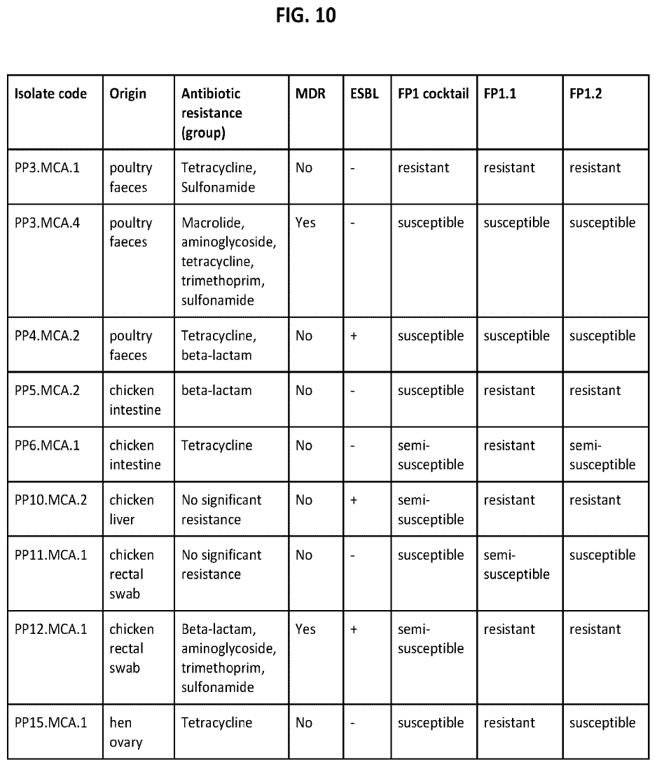

The biocontrol compositions of the invention have substantial lytic activity against Escherichia coli. Specifically, the biocontrol compositions show significant lytic activity against ESBL and/or MDR E. coli, preferably the bacteria listed in FIG. 10 (they are susceptible or semi-susceptible to the biocontrol compositions). The biocontrol compositions also show substantial lytic activity against genotypically distant APEC. (See, FIG. 1a)

The biocontrol compositions of the invention might further comprise other antibacterial agents, in particular other bacteriophage(s) having host specificity distinct from the bacteriophages of the invention and/or antibiotics including antibiotics against gram negative bacteria. Antibiotics may include but are not limited to fosfomycin, imipenem, ertapenem, meropenem, amikacin, tetracycline, ciprofloxacin, trimethoprim/Sulfamethoxazole.

The biocontrol composition of the invention may be in various formulations: liquid, semi-solid, solid, encapsulated or lyophilized. Formulations further comprise a suitable carrier or excipient for the active components (bacteriophages and/or their derivatives) acceptable for pharmaceutical, veterinary or environmental use, depending on the application. The biocontrol composition according to the present invention may include any excipient or carrier that preserve the stability and the substantial lytic activity of the bacteriophages.

The biocontrol compositions of the invention may have a bacteriophage concentration between 102 and 1014 PFU/ml, preferably between 106 and 1013 PFU/ml. When combined, concentration for each bacteriophage should remain between 106 and 1013 PFU/ml.

The biocontrol compositions of the invention may be used to kill E. coli in any organism or environment. The biocontrol compositions may be used for decontamination of surfaces and equipment of medical facilities. The biocontrol compositions used for the decontamination of hospital environment and/or equipment may be formulated as aerosols or spray suitable to be widespread through central air conditioning systems or liquid form, among others. If required, the decontaminated environment and/or equipment may be further subjected to UV exposure.

The biocontrol compositions may be used as a medicament, in particular for the treatment of patients infected or at risk of being infected by E. coli. In humans, the most common diseases caused by pathogenic E. coli are urinary tract infection (most commonly UTI) and enteric infection. The biocontrol compositions may be used for the therapeutic treatment or prophylaxis of UTI or enteric infection in patients, administered either alone or in combination with additional bacteriophages and/or antibiotics. Increasing resistance to antimicrobial agents and the emergence of virulent MDR E. coli strains have made bacterial infection management progressively more challenging. Advantageously, the biocontrol compositions may be administered to patients infected or at risk of being infected by at least one MDR E. coli strain disclosed in FIG. 9. The biocontrol compositions may also be administered to patients infected or at risk of being infected by at least one extended-spectrum beta-lactamases (ESBL)-producing MDR E. coli strain selected from the lists disclosed in FIG. 9. Advantageously, the host-range specificity of the biocontrol compositions for pathogenic E. coli may ensure that the most of the patients' microflora remains unaffected.

Excipients or carriers can be added to the biocontrol compositions to make them suitable for specific formulations, applications and/or modes of administration. For example, injectable formulations may be prepared as aqueous solutions, suspensions or emulsions suitable for intravenous, subcutaneous, intradermal or intramuscular injections; oral formulations may be conditioned as pills, capsules or tablets; topical formulations may be prepared as creams, gel, powder or sprays. Respiratory formulations may be formulated as spray for nebulization. The biocontrol compositions may be freeze-dried or spray-dried for storage, if desired.

With the progressive emergence of MDR E. coli and the restrictions on the use of antibiotics in animal husbandry, the biocontrol compositions of the invention provide an efficient and safe means of controlling E. coli infections. Accordingly, the biocontrol compositions of the invention may be used for treatment of livestock and poultry as well as the decontamination of production farms and slaughterhouses and their equipment. In some embodiments, the biocontrol compositions may be administered to poultry as therapeutic treatments or prophylaxis against at least one APEC strain selected from the lists disclosed in FIG. 1a, preferably a ESBL E. coli selected from the lists disclosed in FIG. 10 and/or MDR E. coli selected from the list disclosed in FIG. 10. Excipients or carriers can be added to the compositions to make them suitable for such applications. Formulations can be administered by any convenient route (e.g., orally, through drinking water, feed or special pills, intravenously, rectally and superficially, among others). In a preferred embodiment, the biocontrol compositions intended to treat poultry at risk for APEC infections may be administered orally through drinking water.

The biocontrol compositions may also be used for in methods for treating contaminated water including sewage of cities, hospitals or meat/poultry production facilities, creeks and rivers, water treatment facilities. In some embodiments, the biocontrol compositions can control the propagation or killing of at least one pathogenic E. coli selected from the lists disclosed in FIG. 1b. In some embodiments, the biocontrol compositions can control the propagation or killing of at least one MDR E. coli selected from the lists disclosed in FIGS. 9 and 10.

### Methods of Producing the Biocontrol Compositions

In some embodiments, each bacteriophage of the invention may be propagated in separate batch culture using standard methods. For example, non-pathogenic E. coli such as A1.MCA.1 (deposited under the Accession No. NCIMB44024) or A3.MCA.5 are cultured, infected with the bacteriophages and allowed to incubate for at least 18 hours. Bacterial cells are subsequently removed from the culture (by centrifugation followed by filtration), resulting in a preparation that contains the culture media, and the bacteriophages. The amount of bacteriophages that results from the propagation can be assessed by spot testing dilutions of the propagation against a bacterial lawn, and counting lysis zones at an appropriate dilution to convert them to PFU. Bacteriophages are further purified for uses stated in the invention by tangential flow filtration, and the desired excipient(s) carrier(s) and/or additional antimicrobial agents may be added at this step.

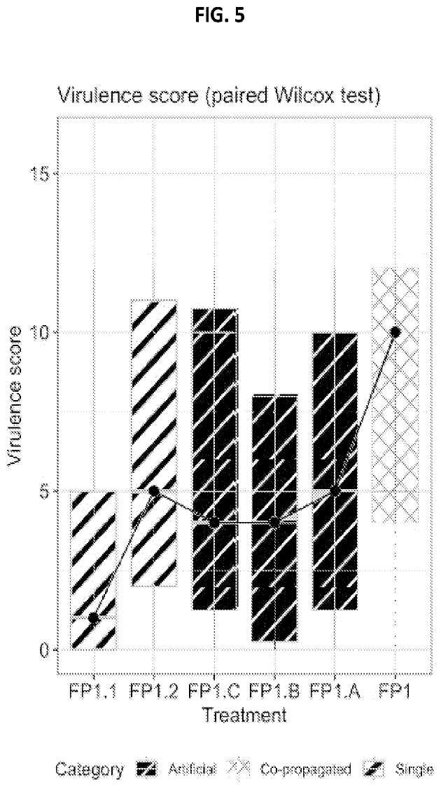

In another embodiment, the bacteriophages are co-propagated in the same batch culture using at least one non-pathogenic E. coli strain such as A1.MCA.1 or A3.MCA.5. Such co-propagation method is not only effective and scalable but importantly reduced production cost and running times. The bacteriophages FP1.1 and FP1.2 can be co-propagated simultaneously in a non-pathogenic E. coli strain such as A1.MCA.1 or A3.MCA.5 in a process that yields a high titer of phages. The bacteriophages remain stable and at their desirable ratios that may vary between 1:1 and 1:14, preferably between 1:2 and 1:6. The presence of both phages and their individual quantification can be verified by deep whole genome sequencing. The co-propagation process was tested in 2 E. coli strains A1.MCA.1 and A3.MCA.5 of different genotypes and origins. Preferably, FP1.1 and FP1.2 are co-propagated at a ratio between 1:2 and 1:6. They all resulted in phage stability across multiple generations, with ratios between the two phages remaining the same; each bacteriophage FP1.1 or FP1.2 retained its host specificity, displayed limited genomic variation between the progeny and the parent or maintained its biological activity. The co-propagation methods of the invention yield to co-propagated cocktails (referred to as FP1, a FP1 cocktail or FP1 biocontrol composition) which have broader host-range than compositions with an individual bacteriophage (See, FIG. 1a, 1b, 9 and FIG. 5). In addition, the co-propagated FP1 cocktail displays a broader host-range than artificial combinations of FP1.1 and FP1.2. (See, FIGS. 5, 6 and 7). As used herein, “artificial combination” refers to bacteriophages propagated in separate batches and subsequently combined in a composition at a desired ratio. In one embodiment, the artificial combination FP1.A results from FP1.1 and FP1.2 produced in separate batch and subsequently combined at equal ratios (1:1). The artificial combination FP1.B results from FP1.1 and FP1.2 produced in separate batch and subsequently combined at a ratio 2:1 respectively. The artificial combination FP1.C results from FP1.1 and FP1.2 produced in separate batch and subsequently combined at a ratio 1:2 respectively.

The invention comprises any combinations of any bacteriophages selected from the group consisting of FP1.1, FP1.2, their variants and their recombinants generated by combining the phages produced by propagation in separate batch culture, or by co-propagating them together in the same batch culture.

### Example

### 1. Materials and Methods

### 1.1. Acquisition of Bacteria

Environmental bacteria were isolated from water samples of rivers and creeks. Samples were plated in MacConkey agar plates and incubated for 24 hours at 37 °C. The next day, pink colonies (lactose-fermenting) were picked and incubated again. This process was repeated until the bacteria were completely isolated. Then, a single colony from each isolate was picked and grown overnight in liquid medium (Brain Heart Infusion Broth, BHI for short) for long term storage with glycerol at −80 °C.

DNA was purified from the isolates and sequenced using the MinION sequencing device from Oxford Nanopore Technologies. The obtained data was analyzed to determine bacterial species, antibiotic resistance, virulence genes, and genotypic classification.

The same process was repeated for the isolation of E. coli from the poultry industry, instead that samples consisted of rectal swabbing, bedding, feces, intestines and other organs. Samples were selected according to clinical signs compatible with E. coli infections, specifically, with APEC infections.

Clinical strains were provided by two hospitals with recurrent problems associated with E. coli infections. Bacteria were identified in the hospitals by trained personnel using MALDI-TOF (mass spectrometry), and their antibiotic resistance determined by standard antibiogram protocols. Upon arrival, bacteria were prepared for long term storage as stated above for the other isolates.

### 1.2. Isolation of Phages

Bacteriophages of the invention were isolated from an environmental water sample as follows: the water sample was filtered with a 0.22 μm of pore size filter and was mixed with an environmental E. coli isolate grown at an OD600 of 2.0 and BHI (Brain Heart Infusion) broth and incubated for 24 hours to enrich for specific bacteriophages. The enrichment was then filtered again with a 0.22 μm of pore size filter to remove residual bacterial cells. The enriched phage solution was plated on BHI agar medium with E. coli embedded. Individual plaques with different morphologies were picked out for further phage propagation and purification.

### 1.3. Propagation of Phages

For primary phage propagation, host bacteria such as non-pathogenic E. coli A1.MCA.1 or A3.MCA.5 were grown at an OD600 of 0.15. An equal amount of bacteria and isolated phage were combined in a rich liquid culture media for propagation (e.g. for preparing 50 ml of a phage propagation solution, 50 μl of bacteria grown at OD 0.15 were combined with 50 μl of isolated phage in a 50 ml of BHI or equivalent culture media). The preparation was incubated for at least 12 hours at 37 °C with constant agitation. After incubation, the propagation solution was further purified by centrifugation of bulk bacterial debris at 6000×g, followed by filtration with a 0.22 μm pore size filter. Propagation solutions were then stored at 4 °C in BHI suspension.

For routine propagation, suitable propagation hosts were selected from the results of the host range determination. Parameters for phage propagation remained the same as for primary propagation, except the amount of phage inoculated varied according to the PFU present per ml in the stock solution.

After propagation, phage titer was quantified using standard methods. Bacterial host was grown at an OD600 of 0.22 and a lawn was created on an agar plate (containing rich medium). Serial 10-fold dilutions of the phage propagations were done in a suitable buffer (from 10¹ to at least 10⁸) and spotted on the dry lawn, and then incubated for 18 hours at 37 °C. PFU values were calculated after.

### 1.4. Co-Propagation

For cocktail co-propagation, host bacteria were grown at an OD600 of 0.15 and then, an equal amount of bacteria and phage cocktail were combined in rich liquid culture media (BHI) for propagation (e.g. for preparing 50 ml of phage propagation, 50 μl of bacteria grown at OD 0.15 were combined with 50 μl of isolated phage in 50 ml of BHI or equivalent culture media). The preparation was incubated for at least 12 hours at 37 °C with constant agitation. After incubation, the propagation was further purified by centrifugation of bulk bacterial debris at 6000×g, followed by filtration with a 0.22 μm pore size filter. Propagations were then stored at 4 °C in a suspension of BHI.

After propagation, the overall cocktail titer was quantified using standard methods. Bacterial host was grown at an OD600 of 0.22 and a lawn was created on an agar plate (containing rich medium). Serial 10-fold dilutions of the phage propagations were done in a suitable buffer (from 10¹ to at least 10⁸) and spotted on the dry lawn, and then incubated for 18 hours at 37 °C. PFU values were calculated after.

### 1.5. Host Range Determination

For determination of the host-range across all strains tested, bacterial candidates were grown to an OD600 of 0.22 and then plated in rich-medium agar plates (BHI) to create lawns. Spots of the phages and their combinations, and dilutions of them (from 10¹ to 10³) were deposited on top of the lawn, and then incubated for 18 hours at an appropriate temperature for the host. Dilutions were plated to rule out toxic or endolysin effects caused by the pure spot.

According to the intensity of clearing and the information provided by dilutions, spots were assigned a score according to the intensity of the clearing, and then, according to them, the strains were classified as susceptible, semi-susceptible or resistant to the phages or phage cocktails.

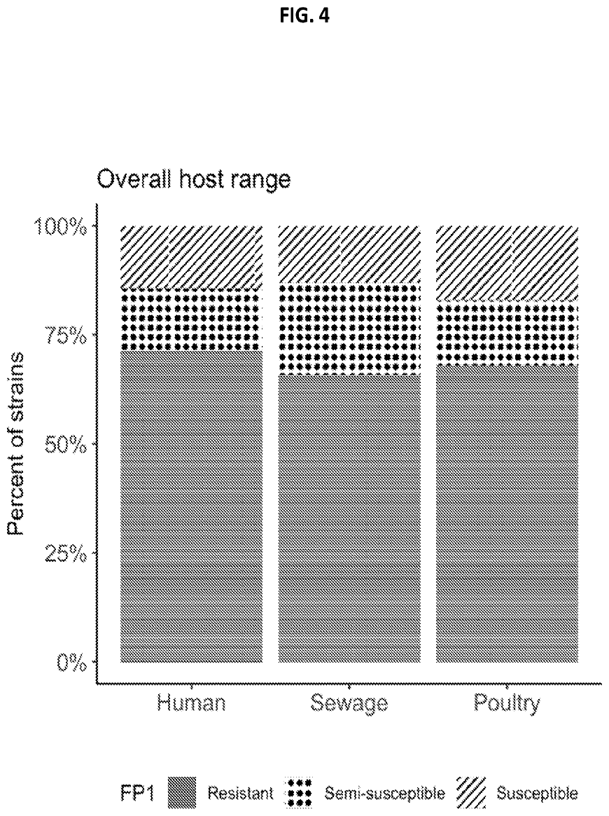

For determination of the differences in host-range between the co-propagated cocktail and artificially generated cocktails, three artificial cocktails were created, by combining different ratios of FP1.1 and FP1.2. The conditions were called FP1.A (equal ratios of both phages), FP1.B (double the amount of FP1.1 than FP1.2) and FP1.C (double the amount of FP1.2 than FP1.1). All these conditions, the co-propagated cocktail and the individual phages were subjected to the host-range analysis using the method described above. Results are shown in FIGS. 4, 5 and 6.

### 1.6. DNA Purification, Sequencing and Analysis

DNA was purified using commercially available silica columns-based DNA purification kits. Purified DNA was checked for quality and quantity, and then sequenced with a MinION device (Oxford Nanopore Technology).

Generated sequencing reads were assembled using Flye assembler, and then a BLAST search was done to determine the approximate identity of the assembled genome. To determine if the assembled phages corresponded to new species, the VIRIDIC software was used. Presence of lysogenic genes was screened using Bacphlip. Presence or absence of antibiotic resistance or virulent genes was screened using Abricate with all the databases available. Protein annotation was done using Prokka, with a phage-specific database.

### 1.7. Coliform Load Evaluation in Water Samples and Antimicrobial Activity of FP1 in Water Samples

To evaluate the coliform count of water samples, at least 50 ml of each sample was collected. The water, and 10-fold, 100-fold and 1000-fold dilutions of it (1 ml of each condition) were directly plated on MacConkey agar plates and incubated either at 37 °C (for total coliform count) or 44.5 °C (for thermotolerant coliform count) for 24 hrs. After incubation, dark pink colonies were counted in each dilution and temperature, and total and thermotolerant CFU/ml were determined.

The same test was repeated in liquid EC medium (using standard techniques). Briefly, different amounts of the sample were inoculated in 10 ml of EC medium (10 ml, 1 ml, 0.1 ml and 0.01 ml) in triplicate, with Durham tubes inside. Tubes were incubated 37 °C (for total coliform count) or 44.5 °C (for thermotolerant coliform count) for 24 hrs. After incubation, tubes were observed for turbidity and gas production, and assigned a Most Probable Number (MPN).

For evaluation of the lytic activity of FP1 against E. coli in a coliform contaminated creek, water samples were collected from a previously analyzed creek. Three different conditions were created with different concentrations of FP1 inoculated (1 ml, 0.1 ml and 0.01 ml of FP1 in 10 ml of water) and three negative controls, inoculated with the same amount of phage-free composition. Conditions were left at room temperature and exposed to natural light for 12 hrs in order to mimic the natural conditions in the creek. After 12 hrs, the coliform test described above was repeated.

### 1.8. Evaluation of Resistance of FP1 Cocktail to Different Conditions

To evaluate the survival and infectivity of FP1.1 and FP1.2 when exposed to extreme pH values, different temperatures, and UV light, the phages were first quantified, and approximate PFU/ml were determined. After that, for the pH resistance test, a constant amount of PFU were inoculated in solutions with pH 2.5 to 10. The phages were left inside each solution for different amounts of time, to simulate the passage inside a chicken: at pH 2.5 and 3 for 90 min, 5 for 8 and then 50 min, 7 for 30 and then 60 min, 8 for 25 min, and pH 9 for 10 min. For pH values not covered in this test (pH 6, 4 and 10) the time selected was 10 and 20 min. After incubation, the solutions were diluted for quantification and spotted in a lawn containing A1.MCA.1, also spotting the solutions without phage as controls for bacterial viability at different pH conditions, and the stock solution of FP1.1 and FP1.2 as the control for phage viability, and comparison for loss of PFU/ml. All experiments were carried out in triplicates, and PFU/ml calculated as an average between them.

To evaluate the replication ability of the phages at different temperatures, serial dilutions of the phage stock were created (from 10-1 to 10-8) and spotted in agar plates containing a bacterial lawn of A1.MCA.1. Plates were incubated at 4 different temperatures: 42 °C (internal temperature of chickens), 37 °C (internal temperature of humans), 30 °C (high environmental temperature in summer), and also incubated at room temperature in winter (temperatures fluctuated between 3 °C and 10 °C). After 24 hours of incubation, PFU/ml were determined for each condition. All experiments were carried out in triplicates, and PFU/ml calculated as an average between them.

### 2. Results

### 2.1. Broad Spectrum of Lytic Activity of Individual Phages and Cocktail Compositions

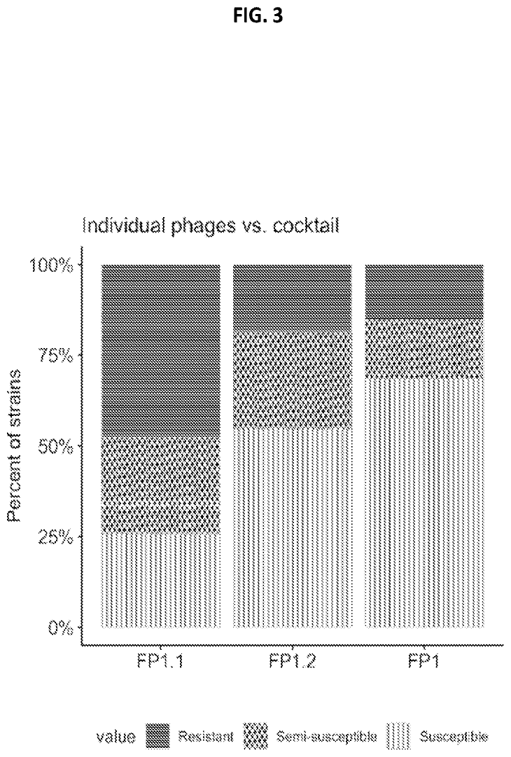

The bacteriophages of the invention were analyzed for host-range against 113 E. coli isolates, selected by their diversity of origin (See, FIGS. 1a, 1b, 9 and 2), genotype, and pathogenicity. Of the 113 strains tested, 68.5% are susceptible to the FP1 cocktail (i.e., they are completely killed), 17.5% are semi-susceptible (i.e., some of the cells in the culture die but others survive), and 14.0% are resistant. (See, FIG. 3) As for the individual phages, 25% of all strains tested are susceptible to FP1.1 and 51% to FP1.2 (See, FIG. 3), with several strains being susceptible to both phages (host-range overlap, See, FIGS. 1a, 1b and 9). Accordingly, the overall host-range of the cocktail does not necessarily equal to the sum of the individual phage's host-range; in fact, some strains, are susceptible to the cocktail but semi susceptible or resistant to the individual phages (See, FIGS. 1a, 1b, 9 and 10; for example, C2 in FIG. 9), indicating that the cocktail significantly improves susceptibility.

2.2. Lytic Activity of the Biocontrol Compositions on MDR Isolates from Different Origins

The bacteriophages of the invention were analyzed for host-range against 6 MDR E. coli strains, 2 isolated from poultry and 4 from human samples (See, FIGS. 9 and 10, MDR column). From the MDR strains tested, the cocktail is able to completely kill all of the strains from human origin, and 1 strain isolated from poultry (the other one being semi-susceptible).

2.3. Lytic Activity of the Biocontrol Composition Against Pathogenic E. coli Strains Isolated from Poultry

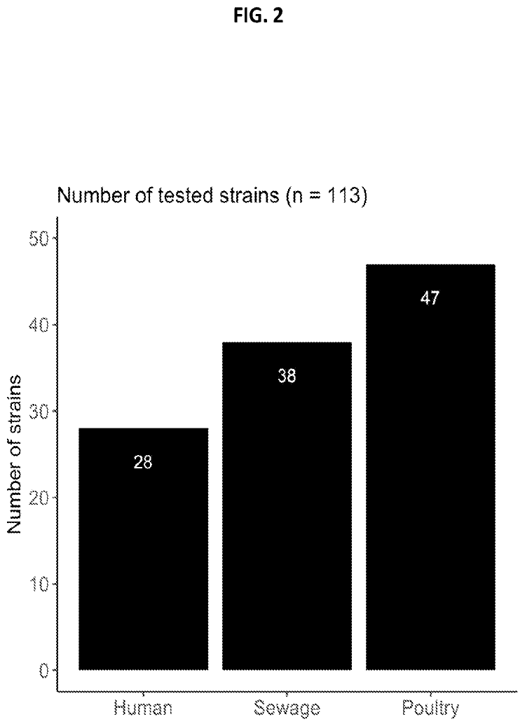

The bacteriophages of the invention were analyzed for host-range against 47 E. coli strains causing disease in poultry (See, FIGS. 1a and 2). Of the strains tested, 9 were APEC (FIG. 10) and belonged to different genotypes; the goal was to show the diversity between APEC strains. Of all the poultry strains tested, 68% are susceptible to the FP1 cocktail, while 17% are semi-susceptible and 15% are resistant (See, FIG. 4). Of the APEC strains tested, 5 out of 9 are susceptible to the FP1 cocktail, while 3 are semi-susceptible, and only one is resistant (See, FIG. 10). Of the susceptible strains, the APEC/MDR strain PP3.MCA.4 is known to pose challenges in poultry facilities (See, FIG. 10). Notably, the FP1 cocktail can efficiently kill PP3.MCA.4 bacteria, further demonstrating its significance for the poultry industry.

### 2.4. Antimicrobial Efficacy of the Biocontrol Compositions on Coliform-Contaminated Water

To test the ability of the FP1 cocktail to eliminate E. coli isolates circulating in water, were the most diversity exists, 38 strains isolated from seven distinct sewage samples were analyzed for host-range (See, FIGS. 1b and 2). Of all the strains tested, 65% are susceptible to the cocktail, 21% are semi-susceptible, and 13% are resistant (FIG. 4).

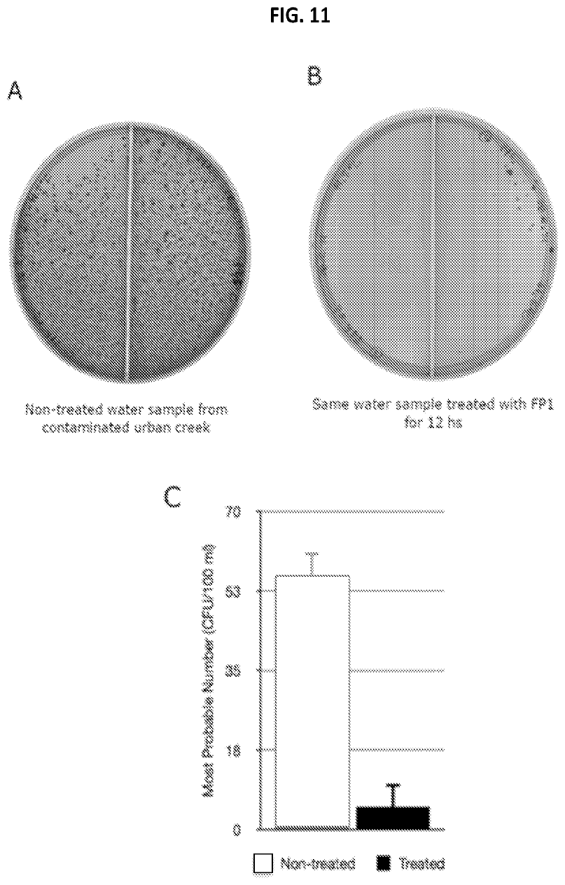

To test whether the cocktail is able to reduce the load of E. coli in a complex environment, such as a coliform-contaminated body of water, a sample from a previously analyzed coliform-contaminated creek was collected. The result of the coliform test with and without cocktail is documented in FIG. 11. After treatment with higher concentration of FP1 cocktail (1/10 dilution, See, materials and methods) a significant reduction of coliform counts was measured. Under non-treated condition, dark coliform-like colonies covered the entire plate. (See, FIG. 11A). In contrast, after FP1 treatment of the contaminated water, only few dark coliform-like colonies remain. (See, FIG. 11B) A quantitative analysis shows a significant reduction of the total CFU/100 ml between the non-treated and FP1-treated condition. (See, FIG. 11C) The same reduction was observed in the liquid medium analysis, thus supporting the effectiveness of FP1 in the treatment of coliform-contaminated water.

2.5. Efficacy of the Biocontrol Compositions on E. coli Associated with Human Infections

The bacteriophages of the invention were analyzed for host-range specificity against 28 pathogenic, disease-related E. coli strains isolated from human infections (See, FIGS. 2 and 9). Of all the strains tested, 72% are susceptible to the FP1 cocktail, 14% are semi-susceptible, and 14% are resistant (See, FIG. 4). As it can be seen in FIG. 9, 6/28 strains tested are ESBL positive, and 5/6 are susceptible to FP1 cocktail, including strains C2, C74 and C75 which are ESBL and MDR positive. Those strains are associated with urinary tract infection (UTI). Based on these results, the FP1 cocktail can be effectively used to treat UTI in human.

### 2.6. Thermal and pH Stability of the Biocontrol Compositions

The bacteriophages of the invention were evaluated for their resistance and ability to replicate in different conditions. Both bacteriophages, FP1.1 and FP1.2 were able to successfully replicate at 42 °C, 37 °C, 30 °C and variable low environmental temperatures without significant loss of PFU/ml with respect to the stock in any of the conditions. Therefore, the bacteriophages can replicate at human and poultry bodily temperature, as well as different environmental temperatures.

With respect their resistance to different pH, both bacteriophages, FP1.1 and FP1.2 were viable and able to replicate without loss of more than 102-fold PFU/ml. (See, FIG. 12) For FP1.1, a slight reduction in PFU could be measured for pH above 7, but not for extreme acidic conditions, such as pH 2.5 and 3. For FP1.2, PFU values remain stable across all conditions, with a reduction for pH values higher than 7. Therefore, both bacteriophages are able to resist and successfully replicate without significant loss of activity at pH ranging from 2.5 to 10, and pH conditions inside poultry.

### 2.7. Improved Virulence of the FP1 Biocontrol Composition Produced by Co-Propagation of the Bacteriophages FP1.1 and FP1.2 in a Single Batch Culture

The bacteriophage compositions of the invention were evaluated for their difference in virulence across all the host-range, according to their ratios and their methods of production. The artificial cocktails FP1.A, FP1.B and FP1.C were prepared by combining FP1.1 and FP1.2, each produced in separate culture batch, at different ratios. These artificial cocktails were compared to compositions of individual phage FP1.1 and FP1.2, as well as the FP1 cocktail produced by co-propagation of FP1.1 and FP1.2 in the same culture batch (See, FIG. 5). Each phage composition was assigned a virulence score for each strain of the host-range tested. (See, FIG. 5) As observed previously, FP1.1 has a lower virulence score, thus a narrower host-range than FP1.2. (See, FIG. 3) Interestingly, artificial cocktails produced from separate culture batch did not show a better score than the individual phages. (See, FIG. 5) Cocktail FP1.C (1:2 ratio of FP1.1:FP1.2) and FP1.B (2:1 ratio of FP1.1:FP1.2) have both lower virulence scores than FP1.2 alone. However, in the case of FP1.A cocktail (equal ratios FP1.1:FP1.2) even though overall host range is better for FP1.2 alone, it has almost the same median value. From these results it can be concluded that artificial combinations do not behave in a predictable way (given that if FP1.2 has a better host range, the artificial combination with a higher ratio of FP1.2, in this case FP1.C, should be the best one of the three, and it is not). Also, the host range of the FP1 cocktail should not be expected to be the sum of the individual host ranges of FP1.1 and FP1.2, as there are many strains that are killed by both phages, and some that are killed by none of them. However, it should be expected for the virulence score to increase significantly with respect to any of the phages alone.

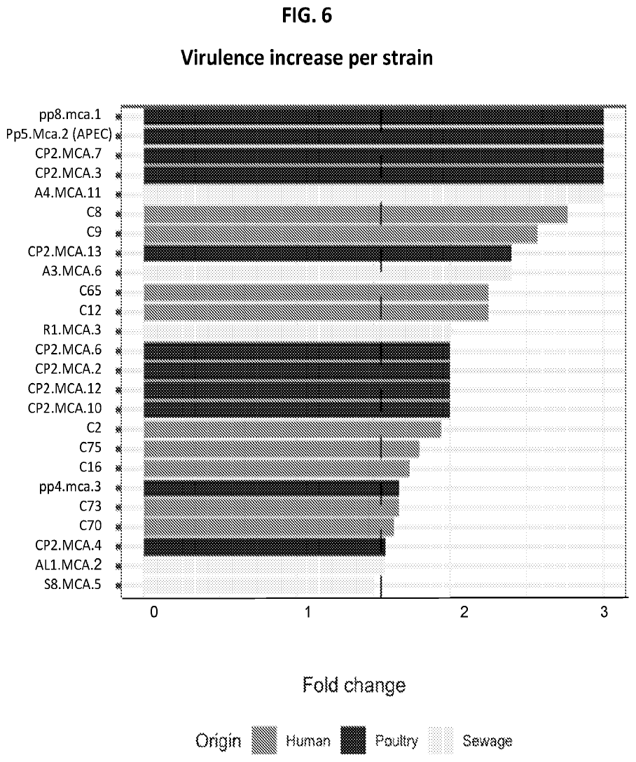

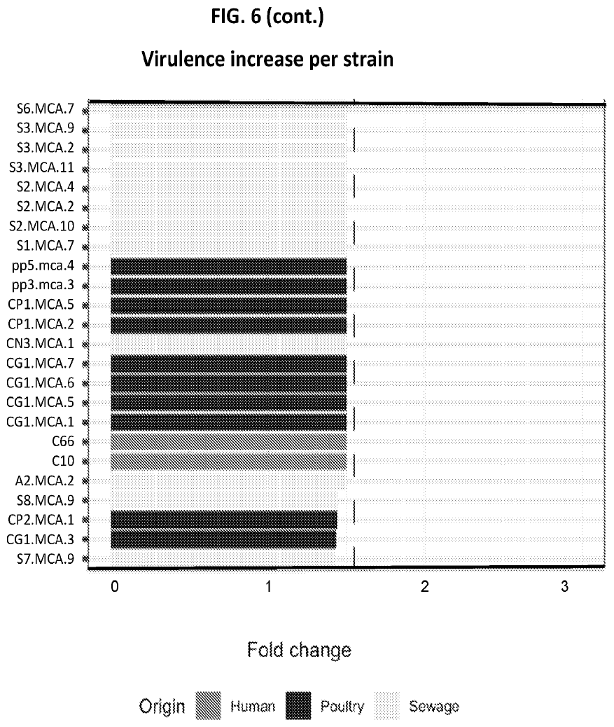

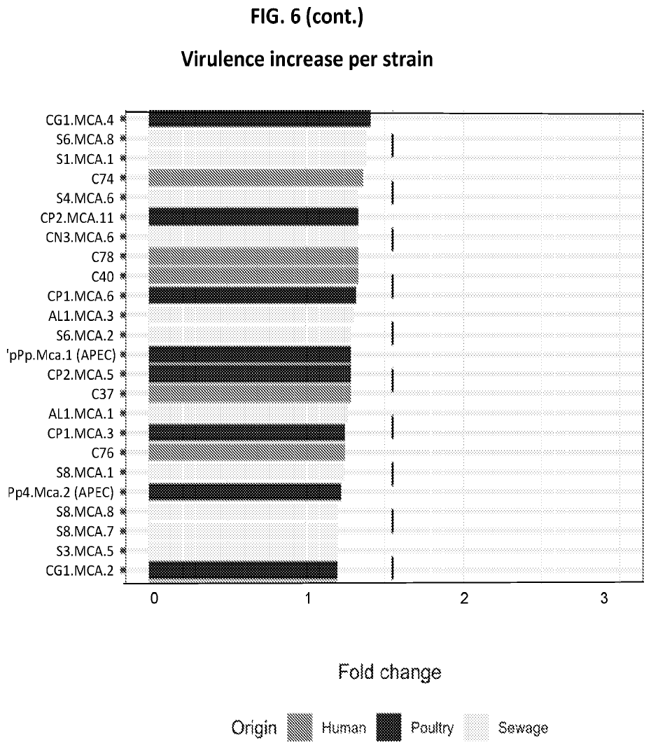

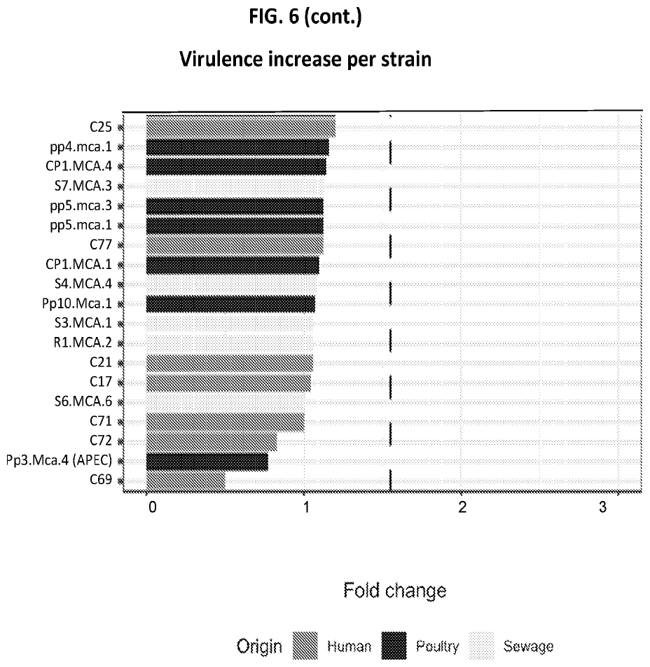

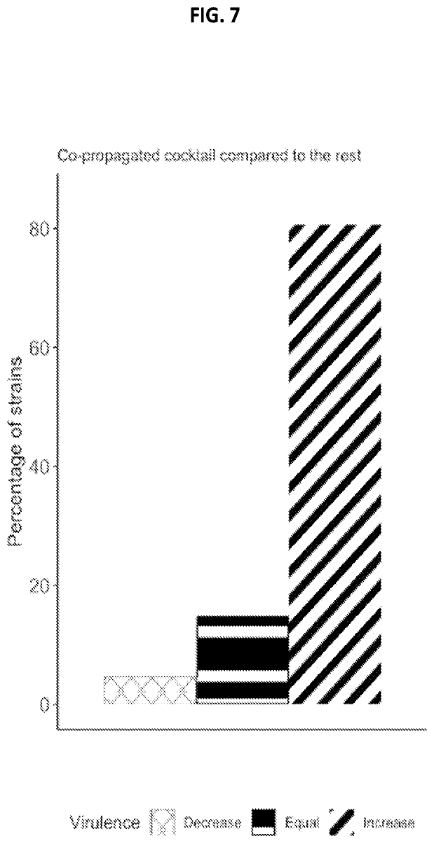

In comparison with the artificial combinations, the FP1 cocktail (co-propagated cocktail) has a significantly higher virulence score that all the other conditions tested, even FP1.2 alone, indicating that the co-propagation process appears to provide an advantage in reaching ideal ratios which results in an improved efficacy of the composition. (See, FIG. 5). In-depth analysis of the virulence change (resulting from comparing the co-propagated cocktail with the rest of the conditions from FIG. 5) on each individual strain is detailed in FIG. 6. For some bacterial strains, the virulence score remains equal for the co-propagated cocktail in comparison with the other conditions, and for a few, it is lower. Nonetheless, the co-propagated cocktail shows significant increase in virulence against most bacterial strains. (See, FIG. 7)

Further aspects of the present disclosure are provided by the subject matter of the following clauses.

A biocontrol composition including at least one distinct bacteriophage having substantial lytic activity against at least one pathogenic bacterial strain, the at least one distinct bacteriophage selected from the group consisting of a bacteriophage having (i). a genome having an overall sequence identity with at least 70% of the genome of the bacteriophage deposited under Accession No. NCIMB44022; (ii). a genome that includes the nucleotide sequence of SEQ ID NO: 1, or a genome including at least 70% overall sequence identity with the nucleotide sequence of SEQ ID NO: 1; (iii). a genome including an overall sequence identity with at least 70% of the genome of the bacteriophage deposited under Accession No. NCIMB44023; and (iv). a genome including the nucleotide sequence of SEQ ID NO: 2, or a genome including at least 70% overall sequence identity with the nucleotide sequence of SEQ ID NO: 2, and a pharmaceutically, veterinary or environmentally acceptable excipient or carrier comprising a preservative in an amount effective to preserve the substantial lytic activity of at least one bacteriophage.

The biocontrol composition of the previous clause including a combination of the at least two distinct bacteriophages, having substantial lytic activity against at least one pathogenic bacterial strain, wherein (i). the first bacteriophage has a genome including an overall sequence identity with at least 70% of the genome of the bacteriophage deposited under Accession No. NCIMB44022, or a genome including the nucleotide sequence of SEQ ID NO: 1, or a genome including at least 70% overall sequence identity with the nucleotide sequence of SEQ ID NO: 1; and (ii). the second bacteriophage has a genome including an overall sequence identity with at least 70% of the genome of the bacteriophage deposited under Accession No. NCIMB44023, or a genome including the nucleotide sequence of SEQ ID NO: 2, or a genome including at least 70% (for example, at least 75%, at least, 80%, a least 85%, at least 90%, at least 91%, at least 92%, at least 93%, at least 94%, at least 95%, at least 96%, at least 97%, at least 98%, or at least 99%) overall sequence identity with the nucleotide sequence of SEQ ID NO: 2.

The biocontrol composition of any preceding clause wherein the first bacteriophage has a genome including an overall sequence identity with at least 75%, at least, 80%, a least 85%, at least 90%, at least 91%, at least 92%, at least 93%, at least 94%, at least 95%, at least 96%, at least 97%, at least 98%, or at least 99% of the genome of the bacteriophage deposited under Accession No. NCIMB44022, or a genome including the nucleotide sequence of SEQ ID NO: 1, or a genome including at least 75%, at least, 80%, a least 85%, at least 90%, at least 91%, at least 92%, at least 93%, at least 94%, at least 95%, at least 96%, at least 97%, at least 98%, or at least 99% overall sequence identity with the nucleotide sequence of SEQ ID NO: 1.

The biocontrol composition of any preceding clause wherein the second bacteriophage has a genome including an overall sequence identity with at least 75%, at least, 80%, a least 85%, at least 90%, at least 91%, at least 92%, at least 93%, at least 94%, at least 95%, at least 96%, at least 97%, at least 98%, or at least 99% of the genome of the bacteriophage deposited under Accession No. NCIMB44023, or a genome including the nucleotide sequence of SEQ ID NO: 2, or a genome including at least 75%, at least, 80%, a least 85%, at least 90%, at least 91%, at least 92%, at least 93%, at least 94%, at least 95%, at least 96%, at least 97%, at least 98%, or at least 99% overall sequence identity with the nucleotide sequence of SEQ ID NO: 2.

The biocontrol composition of the any preceding clause that further includes at least one protein/polypeptide encoded by a nucleic acid sequence selected from the SEQ ID NO: 3 through SEQ ID NO: 964 (see Tables 2 and 3), or at least one catalytic/functional domain derived from the at least one protein/polypeptide thereof.

The biocontrol composition of the any preceding clause wherein the nucleic acid sequence comprises a sequence having at least 70% overall sequence identity with a sequence selected from the SEQ ID NO: 3 through SEQ ID NO: 964.

The biocontrol composition of the any preceding clause wherein the at least one catalytic/functional domain comprises a sequence with at least 70% overall sequence identity with a sequence selected from the SEQ ID NO: 3 through SEQ ID NO: 964.

The biocontrol composition of the any preceding clause that includes additional bacteriophages and/or antibiotics.

The biocontrol composition of the any preceding clause wherein the at least one pathogenic bacterial strain is selected from the family of Enterobacteriaceae.

The biocontrol composition of the any preceding clause wherein the bacterial strain is from the family of Enterobacteriaceae and is selected from the group consisting of the genera Escherichia, Salmonella and Citrobacter.

The biocontrol composition of the any preceding clause wherein the at least one pathogenic bacterial strain is selected from the group consisting of E. coli, E. fergusonii, E. albertii, S. enterica, C. freundii, and P. aeruginosa.

The biocontrol composition of the any preceding clause wherein the at least one pathogenic bacterial strain is an antibiotic-resistant bacterial strain, preferably an extended spectrum beta-lactamase-(ESBL) producing E. coli selected from the lists provided in FIGS. 9 and 10 and/or multi-drug resistant (MDR) E. coli selected from the lists provided in FIGS. 9 and 10.

The biocontrol composition of the any preceding clause which is a liquid, semi-liquid, solid or lyophilized formulation.

The biocontrol composition of the any preceding clause wherein the at least one bacteriophage maintains a substantial lytic activity in at least one of the following conditions:
(i). a temperature between 4 °C and 42 °C and/or;
(ii). a pH between 2.5 and 11 and/or;
(iii). storage at room temperature for a period up to 4 weeks.

A method of controlling at least one pathogenic bacterial strain by exposing said at least one pathogenic bacterial strain to a biocontrol composition of any preceding clause.

The method recited in any preceding clause wherein an effective amount of the biocontrol composition is administered to a susceptible mammal for treating and/or preventing an infection.

The method recited in any preceding clause, wherein the infection is a urinary tract infection or a E. coli-related blood infection affecting a human being, preferably an infection related to at least one of the E. coli strains selected from the list provided in FIG. 9, most preferably an infection related to at least one of the antibiotic-resistant E. coli strains selected from the list provided in FIG. 9.

The method recited in any preceding clause, wherein an effective amount of the biocontrol composition is exposed to an environment or equipment preferably related to a hospital, an animal facility, a slaughterhouse, food processing factory or a water treatment system for decontamination and/or sanitation.

The method recited in any preceding clause wherein an effective amount of the biocontrol composition is added to a contaminated water or wastewater, preferably contaminated with at least one of the E. coli strains selected from the list provided in FIG. 1b. The method recited in any preceding clause wherein the effective amount of the biocontrol composition reduces the concentration of thermotolerant pathogenic coliforms in the contaminated water by at least 70%, preferably 80%, or more preferably by more than 90%.

The method recited in any preceding clause wherein an effective amount of the biocontrol composition is administered to susceptible poultry for treating and/or reducing the risk of avian colibacillosis, and wherein said composition has substantial lytic activity against at least one avian pathogenic E. coli (APEC) strain selected from the list provided in FIG. 1a.

The method recited in any preceding clause, wherein the at least one APEC strain is an antimicrobial resistant bacterial strain, preferably a ESBL E. coli and/or MDR E. coli selected from the list provided in FIG. 10.

The method recited in any preceding clause, wherein the biocontrol composition is formulated as a feed, preferably added to a drinkable liquid.

A method of producing a biocontrol composition having substantial lytic activity against a broad range of pathogenic bacterial strains including the steps of
(a). Maintaining a single non-pathogenic bacteria strain in a vessel
(b). Seeding said single bacterial strain culture with a co-culture of
   (i). a distinct bacteriophage having a genome having an overall sequence identity with at least 70% (for example, at least 75%, at least, 80%, a least 85%, at least 90%, at least 91%, at least 92%, at least 93%, at least 94%, at least 95%, at least 96%, at least 97%, at least 98%, or at least 99%) of the genome of the bacteriophage deposited under Accession No. NCIMB44022, or having a genome including the nucleotide sequence of SEQ ID NO: 1, or a genome including at least 70% (for example, at least 75%, at least, 80%, a least 85%, at least 90%, at least 91%, at least 92%, at least 93%, at least 94%, at least 95%, at least 96%, at least 97%, at least 98%, or at least 99%) overall sequence identity with the nucleotide sequence of SEQ ID NO: 1, and
   (ii). a distinct bacteriophage having a genome having an overall sequence identity with at least 70% (for example, at least 75%, at least, 80%, a least 85%, at least 90%, at least 91%, at least 92%, at least 93%, at least 94%, at least 95%, at least 96%, at least 97%, at least 98%, or at least 99%) of the genome of the bacteriophage deposited under Accession No. NCIMB44023, or having a genome including the nucleotide sequence of SEQ ID NO: 2, or a genome including at least 70% (for example, at least 75%, at least, 80%, a least 85%, at least 90%, at least 91%, at least 92%, at least 93%, at least 94%, at least 95%, at least 96%, at least 97%, at least 98%, or at least 99%) overall sequence identity with the nucleotide sequence of SEQ ID NO: 2,
(c). Harvesting the bacteriophages from the bacterial culture.

The method of the preceding clause, wherein the two distinct bacteriophages are propagated at different ratios ranging between 1:1 and 1:14, preferably between 1:2 and 1:6.

Embodiments of the invention include an isolated nucleotide sequence according to at least one of SEQ ID NO: 3 to SEQ ID NO: 964, or at least one protein/polypeptide encoded by at least one of SEQ ID NO: 3 to SEQ ID NO: 96, or one catalytic/functional domain derived from the at least one protein/polypeptide.

The composition of the preceding clause may be an effective and safe system for delivering a therapeutic agent in host cells.

The composition of any preceding clause relating to at least one protein/polypeptide encoded by at least one of SEQ ID NO: 3 to SEQ ID NO: 964, wherein the protein/polypeptide, alone or in combination with other proteins, may assemble into a nanostructure to deliver the therapeutic agent.

The composition of any preceding clause relating to at least one protein/polypeptide encoded by at least one of SEQ ID NO: 3 to SEQ ID NO: 964, wherein the protein/polypeptide may be a tubulin PhuZ (SEQ ID NO: 874) and nuclear shell protein (SEQ ID NO: 889) derived from bacteriophage FP1.2.

The composition of any preceding clause relating to an isolated nucleotide sequence according to at least one of SEQ ID NO: 3 to SEQ ID NO: 964, or at least one protein/polypeptide encoded by at least one of SEQ ID NO: 3 to SEQ ID NO: 964 may be a biocontrol composition which can be used as a phage-based nanocarrier for the delivery of a therapeutic agent such as nucleic acid therapeutic agent or a drug into a prokaryotic or eukaryotic cell.

The composition of any preceding clause relating to an isolated nucleotide sequence according to at least one of SEQ ID NO: 3 to SEQ ID NO: 964, or at least one protein/polypeptide encoded by at least one of SEQ ID NO: 3 to SEQ ID NO: 964 may be a biocontrol composition which includes at least one of genetically/or chemically engineered bacteriophages to facilitate the delivery and/or targeting of a therapeutic agent into a prokaryotic or eukaryotic cell.

The composition of any preceding clause relating to an isolated nucleotide sequence according to at least one of SEQ ID NO: 3 to SEQ ID NO: 964, or at least one protein/polypeptide encoded by at least one of SEQ ID NO: 3 to SEQ ID NO: 964 may be a biocontrol composition whose nucleic acid therapeutic agent is a functional DNA polymerase or a component of a CRISPR-Cas system.

The composition of any preceding clause relating to an isolated nucleotide sequence according to at least one of SEQ ID NO: 3 to SEQ ID NO: 964, or at least one protein/polypeptide encoded by at least one of SEQ ID NO: 3 to SEQ ID NO: 964 may be a composition which includes at least of the nucleic acid sequence encoding for a functional DNA polymerase involved in DNA replication, DNA repair or genetic recombination. Such composition may be used in vitro as a tool in genetic engineering.

The composition of any preceding clause relating to an isolated nucleotide sequence according to at least one of SEQ ID NO: 3 to SEQ ID NO: 964, or at least one protein/polypeptide encoded by at least one of SEQ ID NO: 3 to SEQ ID NO: 964 may be a composition which includes at least of the nucleic acid sequence encoding for at least one component of a CRISPR-Cas system.

The composition of any preceding clause relating to an isolated nucleotide sequence according to at least one of SEQ ID NO: 3 to SEQ ID NO: 964, or at least one protein/polypeptide encoded by at least one of SEQ ID NO: 3 to SEQ ID NO: 964 may be a composition used for in vivo and in vitro gene editing, including but not limited to point mutations, frameshift mutations, fragment insertions, gene deletions, and gene replacements.

The composition of any preceding clause relating to an isolated nucleotide sequence according to at least one of SEQ ID NO: 3 to SEQ ID NO: 964, or at least one protein/polypeptide encoded by at least one of SEQ ID NO: 3 to SEQ ID NO: 964 the host cell may be a eukaryotic cell.

A method of using a composition as described in any preceding clause relating to an isolated nucleotide sequence according to at least one of SEQ ID NO: 3 to SEQ ID NO: 964 or at least one protein/polypeptide encoded by at least one of SEQ ID NO: 3 to SEQ ID NO: 964.

The embodiments illustrated and discussed in this specification are intended only to teach those skilled in the art the best way known to the inventors to make and use the invention. Nothing in this specification should be considered as limiting the scope of the present invention. All examples presented are representative and non-limiting. The above-described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described.

1. A biocontrol composition comprising at least one distinct bacteriophage having substantial lytic activity against at least one pathogenic bacterial strain, said at least one distinct bacteriophage selected from the group consisting of a bacteriophage having
(i). a genome comprising an overall sequence identity with at least 70% of the genome of the bacteriophage deposited under Accession No. NCIMB44022;
(ii). a genome comprising the nucleotide sequence of SEQ ID NO: 1, or a genome comprising at least 70% overall sequence identity with the nucleotide sequence of SEQ ID NO:1;
(iii). a genome comprising an overall sequence identity with at least 70% of the genome of the bacteriophage deposited under Accession No. NCIMB44023; and
(iv). a genome comprising the nucleotide sequence of SEQ ID NO: 2, or a genome comprising
at least 70% overall sequence identity with the nucleotide sequence of SEQ ID NO:2, and a pharmaceutically, veterinary or environmentally acceptable excipient or carrier comprising a preservative in an amount effective to preserve the substantial lytic activity of the at least one bacteriophage.

2. The biocontrol composition according to claim 1, comprising a combination of at least two distinct bacteriophages, having substantial lytic activity against at least one pathogenic bacterial strain, wherein
(i). a first bacteriophage has a genome comprising an overall sequence identity with at least 70% of the genome of the bacteriophage deposited under Accession No. NCIMB44022, or a genome comprising the nucleotide sequence of SEQ ID NO: 1, or a genome comprising at least 70% overall sequence identity with the nucleotide sequence of SEQ ID NO: 1; and
(ii). a second bacteriophage has a genome comprising an overall sequence identity with at least 70% of the genome of the bacteriophage deposited under Accession No. NCIMB44023, or a genome comprising the nucleotide sequence of SEQ ID NO: 2, or a genome comprising at least 70% overall sequence identity with the nucleotide sequence of SEQ ID NO: 2.

3. The biocontrol composition according to claim 2, wherein the at least 2 distinct bacteriophages are co-propagated in the same culture batch, preferably with at least one strain of non-pathogenic E. coli.

4. The biocontrol composition of claim 1, comprising at least one protein/polypeptide encoded by a nucleic acid sequence selected from the SEQ ID NO: 3 through SEQ ID NO: 964, or at least one catalytic/functional domain derived from the at least one protein/polypeptide thereof.

5. The biocontrol composition of claim 4, wherein the nucleic acid sequence comprises a sequence having at least 70% overall sequence identity with a sequence selected from the SEQ ID NO: 3 through SEQ ID NO: 964.

6. The biocontrol composition of claim 4, wherein the at least one catalytic/functional domain comprises a sequence with at least 70% overall sequence identity with a sequence selected from the SEQ ID NO: 3 through SEQ ID NO: 964.

7. The biocontrol composition according to claim 1, further comprising additional bacteriophages and/or antibiotics.

8. The biocontrol composition according to claim 1, wherein the at least one pathogenic bacterial strain is selected from the family of Enterobacteriaceae.

9. The biocontrol composition according to claim 8, wherein the bacterial strain is from the family of Enterobacteriaceae and is selected from the group consisting of the genera Escherichia, Salmonella and Citrobacter.

10. The biocontrol composition according to claim 8, wherein the at least one pathogenic bacterial strain is selected from the group consisting of E. coli, E. fergusonii, E. albertii, S. enterica, C. freundii, and P. aeruginosa.

11. The biocontrol composition according to claim 10, wherein the at least one pathogenic bacterial strain is an antibiotic-resistant bacterial strain, preferably an extended spectrum beta-lactamase-(ESBL) producing E. coli selected from the strains listed in FIG. 9 and FIG. 10 and/or multi-drug resistant (MDR) E. coli listed in FIG. 9 and FIG. 10.

12. The composition according to claim 8 which is a liquid, semi-liquid, solid or lyophilized formulation.

13. The biocontrol composition according to claim 8, wherein the at least one bacteriophage maintains a substantial lytic activity in at least one of the following conditions:
(i). a temperature between 4 °C and 42 °C and/or;
(ii). a pH between 2.5 and 11 and/or;
(iii). storage at room temperature for a period up to 4 weeks.

14. A method of controlling at least one pathogenic bacterial strain comprising exposing said at least one pathogenic bacterial strain to a biocontrol composition comprising a mixture of at least two distinct bacteriophages, wherein
(i). a first bacteriophage has a genome comprising an overall sequence identity with at least 70% of the genome of the bacteriophage deposited under Accession No. NCIMB44022, or a genome comprising the nucleotide sequence of SEQ ID NO: 1, or a genome comprising at least 70% overall sequence identity with the nucleotide sequence of SEQ ID NO: 1; and
(ii). a second bacteriophage has a genome comprising an overall sequence identity with at least 70% of the genome of the bacteriophage deposited under Accession No. NCIMB44023, or a genome comprising the nucleotide sequence of SEQ ID NO: 2, or a genome comprising at least 70% overall sequence identity with the nucleotide sequence of SEQ ID NO: 2.

15. The method according to claim 14, wherein an effective amount of the biocontrol composition is administered to a susceptible mammal for treating and/or preventing an infection.

16. The method according to claim 15, wherein the infection is a urinary tract infection or a E. coli-related blood infection affecting human, preferably an infection related to at least one of the E. coli strains selected from the list provided in FIG. 9, most preferably an infection related to at least one of the antibiotic-resistant E. coli strains selected from the list provided in FIG. 9.

17. The method according to claim 14, wherein an effective amount of the biocontrol composition is exposed to an environment or equipment preferably related to a hospital, an animal facility, a slaughterhouse, food processing factory or a water treatment system for decontamination and/or sanitation.

18. The method according to claim 17, wherein an effective amount of the biocontrol composition is added to a contaminated water or wastewater, preferably contaminated with at least one of the E. coli strains selected from the list provided in FIG. 1b.

19. The method according to claim 17, wherein the effective amount of the biocontrol composition reduces the concentration of thermotolerant pathogenic coliforms in the contaminated water by at least 70%, preferably 80%.

20. The method according to claim 14, wherein an effective amount of the biocontrol composition is administered to susceptible poultry for treating and/or reducing the risk of avian colibacillosis, and
wherein said composition has substantial lytic activity against at least one avian pathogenic E. coli (APEC) strain selected from the list provided in FIG. 1a.

21. The method according to claim 20, wherein the at least one APEC strain is an antimicrobial resistant bacterial strain, preferably a ESBL E. coli and/or MDR E. coli selected from the list provided in FIG. 10.

22. The method according to claim 20, wherein the biocontrol composition is formulated as a feed, preferably added to a drinkable liquid.

23. Method of producing a biocontrol composition having substantial lytic activity against a broad range of pathogenic bacterial strains comprising
(a). Maintaining a single non-pathogenic bacteria strain in a vessel
(b). Seeding said single bacterial strain culture with a co-culture of
(i). a first distinct bacteriophage having a genome comprising an overall sequence identity with at least 70% of the genome of the bacteriophage deposited under Accession No. NCIMB44022, or having a genome comprising the nucleotide sequence of SEQ ID NO: 1, or a genome comprising at least 70% overall sequence identity with the nucleotide sequence of SEQ ID NO: 1, and
(ii). a second distinct bacteriophage having a genome comprising an overall sequence identity with at least 70% of the genome of the bacteriophage deposited under Accession No. NCIMB44023, or having a genome comprising the nucleotide sequence of SEQ ID NO: 2, or a genome comprising at least 70% overall sequence identity with the nucleotide sequence of SEQ ID NO: 2,
(c). Harvesting the bacteriophages from the bacterial culture.

24. The method according to claim 23, wherein the bacteriophages are propagated at ratio ranging between 1:1 and 1:14, preferably between 1:2 and 1:6 of the first distinct bacteriophage to the second distinct bacteriophage.

25. The biocontrol composition according to claim 2, wherein the at least one pathogenic bacterial strain is selected from the family of Enterobacteriaceae.
